(19) United States
(12) Patent Application Publication
(10) Pub. No.: US 2025/0255687 A1
(43) Pub. Date: Aug. 14, 2025

(54) MEDICAL NANOROBOTS

(71) Applicant: MULTI-SCALE MEDICAL ROBOTICS CENTER LIMITED, Hong Kong (CN)

(72) Inventors: Li ZHANG, Hong Kong (CN); Ben WANG, Shenzhen (CN); Kai Fung CHAN, Hong Kong (CN)

(21) Appl. No.: 19/048,386

(22) Filed: Feb. 7, 2025

Related U.S. Application Data
(60) Provisional application No. 63/550,630, filed on Feb. 7, 2024.

(51) Int. Cl. A61B 34/00; A61B 17/00; A61B 17/22

(52) U.S. Cl. CPC A61B 34/73; A61B 17/22; A61B 2017/00345; A61B 2017/00876; A61B 2017/22084; A61B 2017/22094

(57) ABSTRACT

This invention provides a nanobot, system and method for recanalization of a bodily tube. In one embodiment, said nanorobot comprises: a) a magnetic core; b) a mesoporous shell coated on said magnetic core; and c) an outer layer of a chemical for recanalization bound to said mesoporous shell; wherein said nanobot is adapted to respond to an external magnetic field to carry out motion for mechanical and chemical recanalization.

### FIELD OF THE INVENTION

The present invention relates to medical nanorobots., especially the tPA-anchored nanorobots for in vivo arterial recanalization at submillimeter-scale segments.

### BACKGROUND OF THE INVENTION

Micro/nanorobots provide a promising approach for intravascular therapy with high precision. However, blood vessel is a highly complex system, and performing interventional therapy in those submillimeter segments remains challenging. While micro/nanorobots can enter submillimeter segments, they may still comprise nonbiodegradable parts, posing a considerable challenge for post-use removal. Here, a retrievable magnetic colloidal microswarm, composed of tPA-anchored Fe₃O₄ωmSiO₂ nanorobots (tPA-nbots), was developed to archive tPA-mediated thrombolysis under balloon catheter-assisted magnetic actuation with x-ray fluoroscopy imaging system (CMAFIS). By deploying tPA-nbot transcatheter to the vicinity of the thrombus, the tPA-nbot microswarms were magnetically actuated to the blood clot at the submillimeter vessels with high precision. After thrombolysis, the tPA-nbots can be retrieved via the CMAFIS, as demonstrated in ex vivo organ of human placenta and in vivo carotid artery of rabbit. The proposed colloidal microswarm provides a promising robotic tool with high spatial precision for enhanced thrombolysis with low side effects.

Obstruction of blood vessels (thrombosis) due to blood clots is one of the main causes of death in the world (1). Thromboses are classified on the basis of the site of the thrombus, including ischemic stroke, coronary infarction, and pulmonary embolism. In the early days, people used catheters to remove the blood clots mechanically, especially for acute ischemic stroke, and this process is called thrombectomy. However, thrombolytic therapy through thrombectomy has numerous contraindications, limiting its application to a substantial proportion of patients. For example, the cerebral artery has a lot of branches, including the horizontal/sphenoidal segment (M1), insular segment (M2), opercular segment (M3), and cortical branches (M4). Moreover, with the increase of the branch number, the diameter of the branches diminishes, which makes it difficult to remove the thrombus by thrombectomy. Recently, Zhao and colleagues developed a magnetically steered guidewire enabling telerobotically assisted therapeutic procedures, including clot retrieval at the middle cerebral artery (2, 3). However, as catheter-based devices advance into deeper vessels, their maneuverability and clot retrieval capabilities diminish. Simultaneously, the thin and fragile walls of smaller vessels pose a risk of vascular rupture during mechanical intervention.

Apart from mechanical thrombectomy, thrombolytic therapy using drugs for the dissolution of blood clots is an alternative method for removing blood clots (4-6). Tissue plasminogen activator ((PA), a commonly applied drug for thrombolysis and approved by US Food and Drug Administration (FDA), is a serine protease found on endothelial cells (7). Functioning as an enzyme, tPA can catalyze the conversion of plasminogen (PLG) into activated plasmin (PLM). Subsequently, PLM binds to fibrin and dissolves the blood clot, thereby restoring blood flow. However, a notable mortality rate was noticed at 7 days, as the unrestricted diffusion of tPA within the body poses a risk of symptomatic intracranial hemorrhages (SIHs). SIH is linked to high mortality and poor prognosis for the patient. According to the guidelines for managing patients with acute ischemic stroke published by the American Heart Association Stroke Council (2007) (8), the dosing regimen of intravenous tPA treatment is 0.9 mg/kg with a maximum of 90 mg for each treatment (9). Now, dose control is the primary method for minimizing the risks of SIH after stroke therapy. However, predicting the risk of SIH during thrombolysis treatment remains challenging, contributing to concerns about SIH during thrombolysis treatment. To address this issue, drug delivery carriers have been proposed (10-13). These carriers transport the drug tPA rapidly, efficiently, and with good spatial control to the clot site to recanalize the blood vessels and counteract acute ischemic stroke.

Micro/nanorobots offer a minimally invasive method to address blood vessel obstructions, particularly in small segments deep inside the body, owing to their small size and high motion controllability (14-38). Various drug delivery systems have been investigated to improve spatial control of drug delivery. For example, He and colleagues (16) developed hybrid neutrophil micromotors capable of self-seeking drug delivery in blood vessels through chemotaxis. Engelhard et al. (33) fabricated active nanomotors capable of enhancing tPA-mediated thrombolysis through magnetic field-mediated hydrodynamic convection. Mao and colleagues (17) developed platelet membrane-modified porous silica/platinum nanomotors that can be targeted deep into the thrombus and release drugs through near-infrared light. Zheng and colleagues (18) report a C-shaped magnetic actuation system with improved thrombolysis efficacy by promoting local fluid convection. Compared with the traditional methods, swimming nanorobot-based methods may require less drug (tPA) due to the locally accelerated flow effect. Therefore, the side effects of tPA can be reduced. Although an accelerated tPA release near the blood clot is realized, the increase of the thrombolysis efficiency is limited, and a portion of tPA diffuses and spreads to other places. Furthermore, the retained tPA and nanorobots in the blood vessels may cause long-term side effects, and the removal of the tPA and the nanorobots after treatment may be a good strategy. Iacovacci et al. (20) developed a magnetic device with microchannels that can be embedded in vessels for collection of the free nanoagents. However, considerable nanoagents have already accumulated in various organs before arriving at the collection device. In addition, the devices require a long-period embedding that may cause the ischemia of the tissues at the downstream vessel branches.

In this work, a kind of retrievable magnetic colloidal microswarm, composed of tPA-anchored Fe₃O₄ωmSiO₂ nanorobots (tPA-nbots), was developed to achieve in vivo-enhanced tPA-mediated thrombolysis with an ultralow dose of tPA under a balloon catheter-assisted therapeutic intervention system, in both the veins [using male Sprague-Dawley (SD) rats] and arteries (using New Zealand white rabbit). In the procedure, the tPA-nbots were stored inside a catheter and deployed after the catheter reached the vicinity of the thrombus. The blood flow can be manually blocked by spreading open of the catheter balloon. Then, the tPA-nbot microswarm was steered toward the thrombus site at the submillimeter-scale segments (M3/M4) with magnetic actuation and fluoroscopic imaging-guided real-time tracking. The coupling of mechanical interaction and chemical lysis leads to the highly efficient dissolution of the blood clots, which is reduced by about 42-fold compared with pure tPA, yet the thrombolysis rate is improved about 20 times. After the treatment, the tPA-nbots can be navigated back to the vicinity of the catheter (i.e., its tip), and the vast majority (viz., 80%) of the tPA-nbots can be retrieved and collected. The proposed colloidal microswarm with high spatial precision provides a promising robotic tool to achieve enhanced thrombolysis efficiency, while the side effects and treatment time are greatly minimized.

### SUMMARY OF THE INVENTION

This invention provides a nanobot for recanalization of a bodily tube. In one embodiment, said nanorobot comprises: a) a magnetic core; b) a mesoporous shell coated on said magnetic core; and c) an outer layer of a chemical for recanalization bound to said mesoporous shell; wherein said nanobot is adapted to respond to an external magnetic field to carry out motion for mechanical and chemical recanalization.

This invention also provides a system for recanalization of a bodily tube. In one embodiment, said system comprises: a) a plurality of the nanobots of this invention; b) a delivery mechanism for delivering said plurality of nanobots to said bodily tube; and c) a magnetic source adapted to provide an external magnetic field for controlling motion of said plurality of nanobots.

This invention further provides a method for recanalization of a bodily tube using the system of this invention. In one embodiment, said method comprises the steps of: a) Introducing said plurality of nanobots to vicinity of a recanalization site within said bodily tube using said delivery mechanism; b) Directing said plurality of nanobots to contact said recanalization site using from said magnetic source; and; c) Allowing said plurality of nanobots to mechanically interact with said recanalization site in a rotating magnetic field from said magnetic source.

### BRIEF DESCRIPTION OF THE FIGURES

FIGS. 1A to 1G: Conceptual drawing of the x-ray fluoroscopy-guided delivery of tPA-nbots for targeted thrombolysis and in situ tPA-nbots retrieval using a catheter-assisted magnetic actuation with x-ray fluoroscopy imaging system (CMAFIS). Top schematic was created using BioRender.com.

FIGS. 2A to 2K: Fabrication of the tPA-nbots with enzymatic activity. (A) Schematic showing the fabrication procedure of tPA-nbots. (B and C) transmission electron microscopy images of the tPA-nbots with low (B) and high magnification (C) (tPA-nbot size, ˜300 nm). (D) Thermogravimetric analysis of the pure tPA and tPA-nbots. (E) Fluorescence microscopy image of tPA-nbots coupled with fluorescein isothiocyanate (FITC) and exposed to a magnetic field. (F) Relationship between the fluorescence intensity and the amount of tPA-Fitc (solution volume is 200 μl). (G) Bar graph showing the fluorescence intensity of the pure nanorobots, tPA-FITC with dose of 0.303 and 10 μg, and tPA-FITC-grafted nanorobots (solution volume is 200 μl). (H and I) Assessment results of the activity of tPA on the tPA-grafted nanorobots. (H) The curves show the course of the optical density (OD) value at 405-nm over the reaction time at different concentrations of tPA and tPA-nbots. (I) Relationship between the OD value and the concentrations of tPA-nbots after a reaction time of 128 min. (J and K) Cell viability of mesenchymal stem cell and human umbilical vein endothelial cells, respectively, after incubation with nanorobots (24 hours).

FIGS. 3A to 3I: Trapping and retrieval efficiency of the nanorobots swarm under various parameters in an in vitro branched vessel model. (A) Schematic showing the retrieval capability of the tPA-nbots (see the photo of experimental setup in FIGS. 19A to 19E). (B) Trapping percentage of the nanorobots versus the injection dose of the nanorobots. (C) Trapping percentage of the nanorobots versus the concentration of the nanorobots. (D) Trapping percentage versus injection speed of the nanorobots. (E) Trapping percentage versus magnetic field strength. (F) Trapping percentage versus the magnetic navigation distance of the nanorobots inside vessel after they are released from the catheter. (G) Trapping percentage versus simulated flow speed in the vessel. (H) Retrieval percentage by the catheter after nanorobots delivery versus the simulated flow speed in the vessel. (I) Simulation result showing the capture of the nanorobots with external magnetic field in aqueous solution.

FIGS. 4A to 4F: Mechanism of thrombus removal by using the tPA-nbots. (A) Snapshots showing the interaction between blood clot and nanorobots+tPA in contact mode, nanorobots+tPA in noncontact mode, and solely tPA. (B) Bar graph showing the thrombolysis rate of the systems in (A). (C) Snapshots showing the chemical etching of tPA-grafted nanorobots with RMF, nanorobots with RMF, and tPA-grafted nanorobots without RMF. (D) Bar graph showing the thrombolysis rate of the systems in (C). (E) Schematic showing the chemical etching and mechanical interaction by using the tPA-nbots. (F) Simulation result showing the flow field of the nanorobot chains in a confined channel with a diameter of 1.5 mm.

FIGS. 5A to 5N: Catheter-assisted magnetic navigation of tPA-nbots enables the fluoroscopy-guided thrombolysis in human placentas with the ability to retrieve the tPA-nbots. (A) Photograph of the experimental setup containing magnetic control unit, fluoroscopy, and catheter. (B) Photograph and computed tomography image showing the vascular distribution of the human placenta. (C) Photographs showing the human placenta with an artificial blood clot at a 1.5-mm branched vessel. (D) Photographs showing the human placenta after the blood clot is removed with the tPA-nbots with retrieved manner. (E) Schematic of stage 1: catheter deployment. (F) Stage 1: insertion of a catheter at the vessel that is in close vicinity to the vessel with the blood clot, and release of the tPA-nbots while real-time tracking by fluoroscopy imaging is conducted. (G) Schematic of stage 2: controlled thrombolysis. (H) Stage 2: magnetic navigation of the tPA-nbots toward the blood clot for thrombolysis while real-time tracking by fluoroscopy imaging is conducted. (I) Schematic of stage 3: retrieval of nanorobots. (J) Stage 3: guiding the nanobots back to the terminal of the catheter and removal of the nanobots by using the catheter for vascular recanalization, the x-ray tube current automatically adjusted ranging from 21.3 to 33.1 mA, and the voltage was maintained at 70 kV during the whole process. (K to N) Retrieval capability of nanorobots in placenta, with blood flows and multiple branches (movie S6). (K) Retrieval percentage versus nanoparticle dose. (1) Retrieval percentage versus magnetic field strength. (M) Retrieval percentage versus locomotion distance of the tPA-nbots. (n) Retrieval percentage versus flow speed in placenta. the flow speeds given in the x axis are the maximum value in the placenta vessels.

FIGS. 6A to 6I: Reducing the blood flow to enable retrieval of tPA-nbots by the catheter balloon. (A) The photo shows the blood vessel diameter distribution of the placenta used in this experiment. (B) Selected blood vessels of the placenta used in this experiment, including main branch 1, main branch 2, main branch 3, distal branch 1, and distal branch 2. (C) Real image and pseudo-color patterns of the blood vessel with or without catheter balloon intervention. (D to F) the change of the perfusion unit, flow, and flow velocity with or without the catheter balloon intervention. (G) The photos exhibit the whole process of targeted locomotion and retrieval of the tPA-nbots with the catheter balloon intervention. (H) Photographs illustrating the effective entrapment of tPA-nbots facilitated by catheter balloon intervention, contrasting with the total loss of tPA-nbots in the absence of catheter balloon intervention under conditions of elevated blood flow. (I) The retrieval rate of the tPA-nbots with or without catheter balloon intervention.

FIGS. 7A to 7E: In vivo thrombolysis with rat model. (A) The photos show the blood clot formation process using a fabric film soaked with 5 wt % FeCl₃ solution in femoral vein of rat. (B) The average recanalization time of the occlusive femoral vein after treated by tPA-labeled microswarm. Magnetic field strength, 64.9 mT; frequency, 3 Hz; dose of tPA-nbots, 0.5 mg. (C) The imaging of whole treatment process, including blood clot inducing process, swarm delivery process, and thrombolysis, which was captured by LSBFIS. Pseudo-color pattern (left image) and gray pattern (right image) were recorded for analysis. (D) Quantitative analysis of the perfusion rate of position 1 and position 2. (E) The imaging of control groups, including group 1, PBS; group 2, nanorobots group; group 3, tPA; group 4, nanorobots+tPA; and group 5, tPA-anchored nanorobots without RMF.

FIGS. 8A to 8I: Catheter-assisted magnetic navigation of tPA-nbots enables the fluoroscopy-guided thrombolysis in New Zealand white rabbit. (A) Photograph of the experimental setup containing magnetic control unit, fluoroscopy, and catheter. (B) Schematic and photographs showing the generation of an artificial blood clot at a 0.9-mm carotid artery and catheterization of a 1.1-mm catheter through the femoral artery of the rabbit. created using BioRender.com. (C) Step 1: catheter insertion. (D) Step 2: nanorobots delivery and release. (E) Step 3: controllable locomotion of nanorobots toward the clot. (F) Step 4: continuous thrombolysis. (G) Step 5: controllable locomotion of nanorobots toward the catheter. (H) Step 6: retrieval of nanorobots by catheter. (I) Step 7: clinical imaging contrast injection.

FIGS. 9A to 9E: XPS spectra showing the chemical composition of the Fe₃O₄ωmSiO₂ composite, Fe₃O₄ωmSiO₂-bis-NHS-PEG6, and Fe₃O₄ωmSiO₂-tPA nanorobots (tPA-nbots). (A) Comprehensive XPS spectrum. (B) Detailed XPS spectrum of C 1s. (C) Detailed XPS spectrum of Fe 2p. (D) Detailed XPS spectrum of N 2p. (E) Detailed XPS spectrum of Si 2p.

FIG. 10: FTIR spectrum shows the surface functional groups of Fe₃O₄ωmSiO₂ composite, Fe₃O₄ωmSiO₂-bis-NHS-PEG6, and Fe₃O₄ωmSiO₂-tPA nanorobots (tPA-nbots).

FIGS. 11A to 11E: Assembly of the particles to microrods under magnetic field. (A) Schematic illustration of the assembly of the particles to microrods under magnetic field. (B) SEM image shows the original nanospheres without magnetic field assembly. (C) SEM images show the integral and enlarged views of microrods with magnetic field assembly. The inset shows the nanorod alignment on an microrod. (D) TEM images show the configuration of microrod after magnetic field assembling. (E) Bright field and fluorescent images show the alignment of the nanospheres along the direction of magnetic field.

FIG. 12: Magnetic hysteresis loops of the Fe₃O₄ microspheres, Fe₃O₄ωmSiO₂ composite, and Fe₃O₄ωmSiO₂-tPA nanorobots (tPA-nbots) at 300 K by VSM.

FIGS. 13A to 13D: tPA-nbots diameter measurement with dynamic light scattering after the magnetic actuation (strength is 64.9 mT, frequency of 3 Hz) within a simulated vessel with flow speed of 5 cm/s for 5 min, 10 min, 30 min, and 60 min, respectively.

FIG. 14: Curves showing the OD value at 405 nm over the reaction time for the residual solution subsequent to the removal of tPA-nbots. The depicted curves illustrate the temporal evolution of the optical density (OD) at 405 nm throughout the reaction time for the residual solution subsequent to the removal of tPA-nbots (1 mg/mL), which underwent magnetic swarming for different durations (5 min, 10 min, 30 min, 60 min, and 120 min).

FIGS. 15A to 15M: Motion control of the tPA-nbots microswarm. (A) Schematic showing the assembly of the nanorobots during the controlled swarming process. (B) Snapshots showing the controlled swarm generation and disassembly (spreading) of the nanorobots. (C, D) Swarm generation time and area as functions of input frequency at variety of magnetic field strengths. (E) A demo showing the magnetic locomotion of the nanorobot swarm with programmable trajectories, i.e., “CUHK”. (F) Schematic showing the swarm on a flat surface. (G) The phase diagram showing the swarm patterns actuated by different input magnetic field frequencies. (H) The phase diagram showing the swarm patterns actuated by different input magnetic field strengths. (I) Optical images showing the possible pattern states in (G) and (H). (J) Schematic showing the swarm on a curved surface. (K) The phase diagram showing the swarm velocity by different input frequency in a 1.5-mm vessel (particle dose is 0.2 mg). (L) The phase diagram showing the swarm velocity by different input magnetic field strengths in a 1.5-mm vessel (particle dose is 0.2 mg). (M) US images showing the pattern states in (K) and (L).

FIGS. 16A to 16F: Quantitative investigation of the nanorobot loss inside the catheter, before entering the vessel. (A) Real weight of the obtained nanorobots at the outlet of the catheter versus the injection speed of the nanorobots (dose: 3 mg, concentration: 2 mg/mL, diameter of catheter: 1 mm (inner), length of catheter: 150 cm). (B) Calibrated volume versus the injection speed of the nanorobots. With the increase of injection speed, the recycle weight increases, reaching about 2.5 mg when the injection speed is larger than 0.2 mL/s. (C) Real weight of the obtained nanorobots at the outlet of the catheter versus the injection dose of the nanorobots (injection speed: 0.2 mL/s, concentration: 2 mg/mL, diameter of catheter: 1 mm (inner), length of catheter: 150 cm). (D) Calibrated weight versus the injection dose of the nanorobots. More nanorobots are lost in the catheter with the increase of injection dose. (E) Real weight of the obtained nanorobots at the outlet of the catheter versus the injection concentration of the nanorobots (injection speed: 0.2 mL/s, dose: 3 mg, diameter of catheter: 1 mm (inner), length of catheter: 150 cm). (F) Calibrated volume versus the nanorobots concentration. The loss weight is much larger when the concentration exceeds 4 mg/mL.

FIGS. 17A to 17G: Trapping weight and loss weight of the nanorobots swarm under a variety of parameters in an in vitro branched vessel model. (A) Trapping weight and loss weight versus the injection dose of the nanorobots (Injection speed: 0.05 mL/s, Dose: 3 mg, Concentration: 2 mg/mL, Diameter of saline tube: 2.5 mm (inner), Length of catheter: 142 cm, Diameter of catheter: 1 mm (inner), Frequency: 3 Hz, Magnetic strength: 64.9 mT, Distance between catheter tip and magnet center: 20 mm). (B) Trapping weight and loss weight versus the concentration of the nanorobots (Injection speed: 0.05 mL/s, Dose: 3 mg, Concentration: 0.5, 1, 2, 3 4, 5, 6 mg/mL, Diameter of saline tube: 2.5 mm (inner), Length of catheter: 142 cm, Diameter of catheter: 1 mm (inner), Frequency: 3 Hz, Magnetic strength: 64.9 mT, Distance between catheter tip and magnet center: 20 mm) 0.05). (C) Trapping weight and loss weight versus injection speed of the nanorobots (Injection speed: 0.025, 0.05, 0.1, 0.2, 0.3, 0.4 mL/s, Dose: 3 mg, Concentration: 2 mg/mL, Diameter of saline tube: 2.5 mm (inner), Length of catheter: 142 cm, Diameter of catheter: 1 mm (inner), Frequency: 3 Hz, Magnetic strength: 64.9 mT, Distance between catheter tip and magnet center: 20 mm). (D) Trapping weight and loss weight versus magnetic field strength (Injection speed: 0.05 mL/s, Dose: 3 mg, Concentration: 2 mg/mL, Diameter of saline tube: 2.5 mm (inner), Length of catheter: 142 cm, Diameter of catheter: 1 mm (inner), Frequency: 3 Hz, Magnetic strength: 22.4, 37.2, 64.9, 121.5 mT, Distance between catheter tip and magnet center: 20 mm). (E) Trapping weight and loss weight versus the magnetic navigation distance of the nanorobots inside vessel after they are release from the catheter (Injection speed: 0.05 mL/s, Dose: 3 mg, Concentration: 2 mg/mL, Diameter of saline tube: 2.5 mm (inner), Length of catheter: 142 cm, Diameter of catheter: 1 mm (inner), Frequency: 3 Hz, Magnetic strength: 22.4, 37.2, 64.9, 121.5 mT, Distance between catheter tip and magnet center: 1, 2, 3, 4, 5 cm). (F) Trapping weight and loss weight versus simulated flow speed in the vessel (Injection speed: 0.05 mL/s, Dose: 3 mg, Concentration: 2 mg/mL, Diameter of saline tube: 2.5 mm (inner), Length of catheter: 142 cm, Diameter of catheter: 1 mm (inner), Frequency: 3 Hz, Magnetic strength: 64.9 mT, Distance between catheter tip and magnet center: 20 mm). (G) Retrieval weight (finally recycled weight by the catheter after nanorobots delivery) and injected weight (initially injected weight to the vessel) versus the simulated flow speed in the vessel (Injection speed: 0.05 mL/s, Concentration: 2 mg/mL, Dose: 3 mg, Magnetic strength for trapping and locomotion: 64.9 mT, Locomotion distance: 12 cm, Locomotion speed: 1 mm/s, Extraction speed: 0.25 mL/s).

FIGS. 18A to 18C: Mechanical property of blood clot. (A) Curves showing the relationship between the force and the displacement of the blood clots with different incubation time. (B) Stress versus strain of the blood clot with different incubation time. (C) Young's moduli of the blood clots with different incubation time.

FIGS. 19A to 19E: In vitro thrombolysis performance of the tPA-nbots in vessels filled with blood. (A) Images of the thrombolysis process by four different “drugs”. Drug 1:1 mg of tPA-nbots (15 μg of tPA); Drug 2:625 μg of tPA; Drug 3:1 mg of Fe₃O₄ nanorobots; Drug 4: blank control. 20 μL of 0.5 M CaCl₂) solution was added into 1 M of fresh pig blood to generate the artificial blood clot with 15 min incubation. (B) Bar graph showing the thrombolysis speed of the drugs in (A). (C) Relationship between the thrombolysis speed and the dose of tPA grafted nanorobot. (D) Video captures showing the thrombolysis of a long blood clot with large aspect ratio of 11 (field strength: 37.5 mT, frequency: 3 Hz, dose: 1 mg tPA-nbots, tube diameter is 1.5 mm, thrombolysis rate is 0.53 mm³/min). (E) Targeted thrombolysis in a complex 3D channel in diluted blood (field strength: 15.1 mT, input frequency: 3 Hz, dose: 1 mg tPA-nbots (tPA: 15 μg), tube diameter: ˜1.5 mm, temperature: ˜37° C.). For Group 1, 1 mg of tPA-nbots was administered. The dose of tPA on the nanorobots was calculated to 15 μg. The original length of the artificial blood clot was 6.5 mm, and more than half of the clot is degraded by the action of tPA from tPA-nbots after 9 min. It took 18 min to fully remove the blood clot using tPA-nbots, and the thrombolysis rate was around 0.638 mm³/min. In Group 2, the thrombolysis of solely 625 μg tPA was investigated, and it was found to be much lower than that that of tPA-nbots. Only a small part of the blood clot is lysed by the tPA after 18 min, and the thrombolysis rate was calculated to approx. 0.0318 mm³/min.

FIGS. 20A and 20B: Thrombolysis process of the blood clots with different moduli. (A) Thrombolysis process of the blood clots with 15-min incubation (modulus is 1.75 kPa), 30-min incubation (modulus is 2.22 kPa), 60-min incubation (modulus is 3.11 kPa). (B) Thrombolysis rate versus incubation times of the blood clots.

FIGS. 21A to 21C: Simulations of swarm pattern-induced flow profile near a clot-fluid interface. (A) Simulation result of the pattern-induced fluid profile near the interface. The input rotating frequency is 3 Hz. Each chain has a length of 250 μm and a width of 40 μm. Color profile denotes the flow velocity as marked by the color legend (μm/s). (B) Flow velocity distribution between the pattern and the clot-fluid interface t=5 s. The cut lines are parallel to the interface (y axis) and placed at the center position between the interface and pattern's left side. (C) Shear stress distribution along the cut line. The profiles in this plot are reproduced using MATLAB based on the simulation results.

FIGS. 22A to 22D: Simulations of the motion of red blood cells around a swarm pattern. 250 simulated RBCs (6-μm-diameter microparticles) are released from five releasing points (50 particles each) at t=0.2 s. The input rotating frequency is 3 Hz. Color profile denotes the motion velocity of simulated RBCs as marked by the color legend (μm/s). The simulated RBCs are dynamically affected by the swarm pattern.

FIGS. 23A to 23C: In-vivo thrombolysis using tPA-nbots in rats with in-situ induced blood clots in the femoral vein. (a) Optical images show a rat with a blood clot in its femoral vein. (b) experimental set-up for the treatment of the thrombus of a rat with microscopic inspection. (c) Photo images show the cut blood vessel of the thrombus site of the rate without nanorobots and with nanorobots treatment. The right image shows the squeezed blood clot from the corresponding vessels.

FIGS. 24A to 24I: Catheter-assisted magnetic navigation of tPA-nbots for targeted thrombolysis in a 3D in vitro channel. (A) Photograph showing the catheter-assisted magnetic navigation of tPA-nbots for targeted thrombolysis. (B) Enlarged view of the blood clot in the in vitro vascular model (3D-printed by transparent resin). (C) Enlarged view of the blood clot. (D) Schematic showing the catheter insertion and nanorobot release. (E) Optical images showing the insertion of catheter, blockage of the vessel by balloon, and release of the nanorobots. (F) Schematic showing the magnetic field guided thrombolysis. (G) Optical images showing swarming motion of the nanorobots toward the blood clot, and the mechanical interaction and chemical etching process of the blood clot. (H) Schematic showing the recapturing process of the colloidal microswarm. (I) Optical images showing the controlled actuation of the nanorobots back to the catheter, and the suction of the nanorobots for vascular recanalization.

FIGS. 25A and 25B: Thrombolysis rate of tPA-nbots under various magnetic field frequencies. (A) Sequential images illustrating the thrombolysis rate of tPA-nbots under controlled thrombus dimensions (length: 8 mm, diameter: 1.5 mm) and a magnetic field strength of 65 mT, across a range of frequencies (1 Hz, 3 Hz, 5 Hz, 7 Hz, and 9 Hz). (B) Bar graph depicting the thrombolysis rate of tPA-nbots under varying magnetic field frequencies.

FIG. 26: Optical images showing the state of the blood clot before and after tPA release. The catheter was used for delivering tPA, and the catheter tip was at the site with a distance of 40 mm to the blood clot. The tPA dose used here was 1.5 mg, which is 100 folds compared with the tPA dose of 1 mg nanorobots. Blood: 4× diluted, blood flow: 75 mm/s. Scale bar: 5 mm.

FIGS. 27A and 27B: Magnetic navigation of tPA-nbots under an ex vivo model of pig head by fluoroscopic tracking. (A) Photographs showing the setup. (B) X-ray fluoroscopic images showing the magnetic actuation under an ex vivo model of pig. The nanorobot collectives can be tracked as an entity due to the milli-scale spatial resolution of the fluoroscopy with exceptional penetration capability of pig skull. (Dose: 5 mg, magnetic strength for trapping and locomotion: ˜ 37 mT, frequency: 3 Hz, flow speed: 10 mm/s, blood: whole blood, imaging contrast: 100 mg/mL)

FIG. 28: Snapshots showing the nanorobots delivery, locomotion, and retrieval process, tracked by fluoroscopy imaging in real-time manner (Injection speed: 0.05 mL/s, concentration: 2 mg/mL, dose: 5 mg, magnetic strength for trapping and locomotion: 64.9 mT, locomotion distance: 12 cm, locomotion speed: 1 mm/s, flow speed: 4 cm/s).

FIG. 29: Imaging contrast of microswarm with different blood flows. (A) The schematic shows the setup for in vitro fluoroscopy imaging of microswarm, consisting of a pump, a silicone tube, a magnet, and a motion controller. (B-F) The snapshots show the fluoroscopy imaging of microswarm (3 mg) in the tube with whole blood and different flow speeds (0, 2, 6, 10, 14 cm/s). Downstream and upstream locomotion speed: 0.5 mm/s, magnetic field strength: 121.5 mT, frequency: 3 Hz. The microswarm showed good imaging contrast in an in vitro model. There is no significant decline in the imaging effect of microswarm with different blood flows. The microswarm can move against a high blood flow of 14 cm/s with larger magnetic field strength.

FIG. 30: Photographs showing the representative placentas used in experiments. The placenta is similar to the human brain both in structure and components, consisting of soft tissue as well as complicated and tortuous blood vessels, which can be regarded as an ideal model to simulate a stroke in the human brain. The placentas show complex and torturous blood vessel distribution, and the diameter of the blood vessel ranges from 1.0 mm to 5.5 mm.

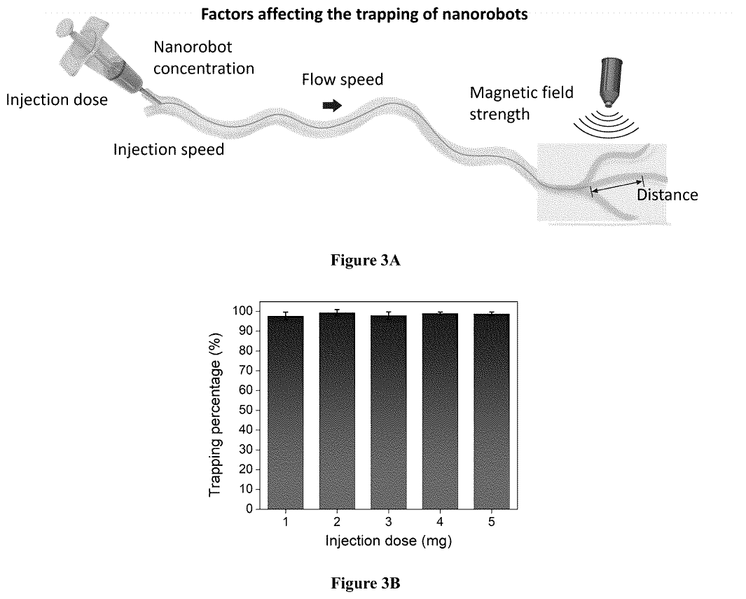

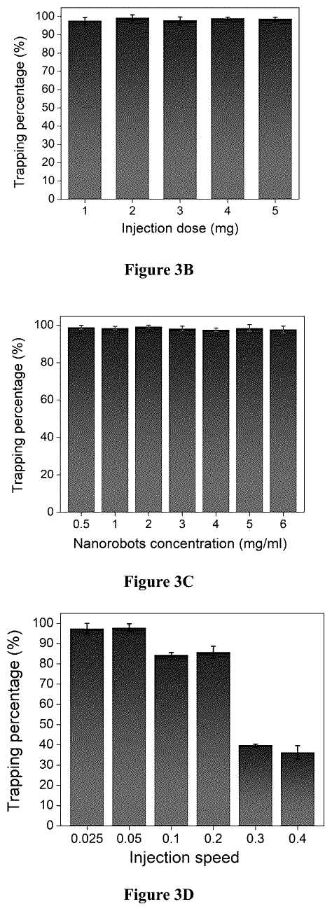

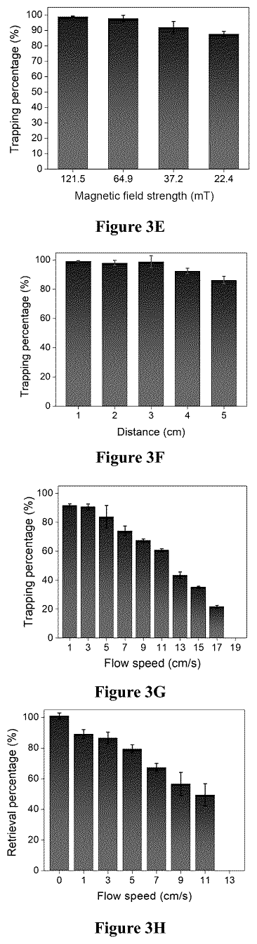

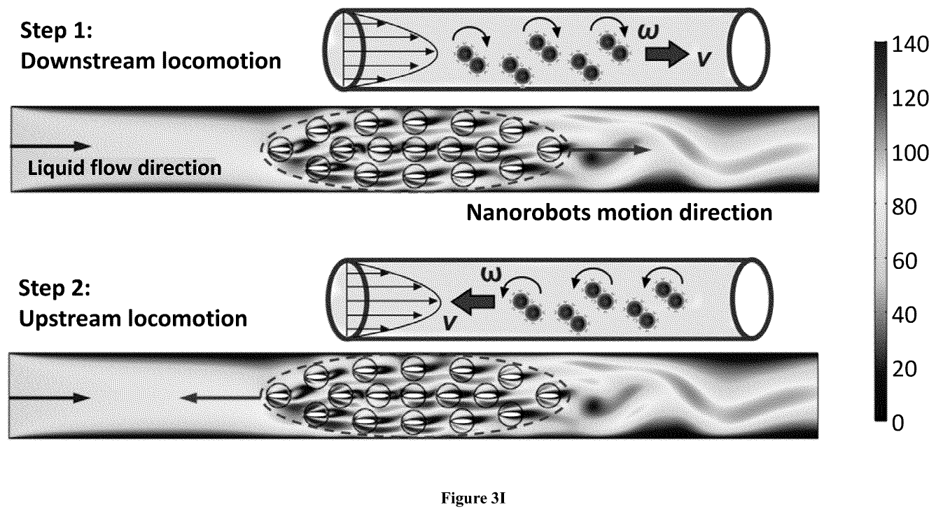

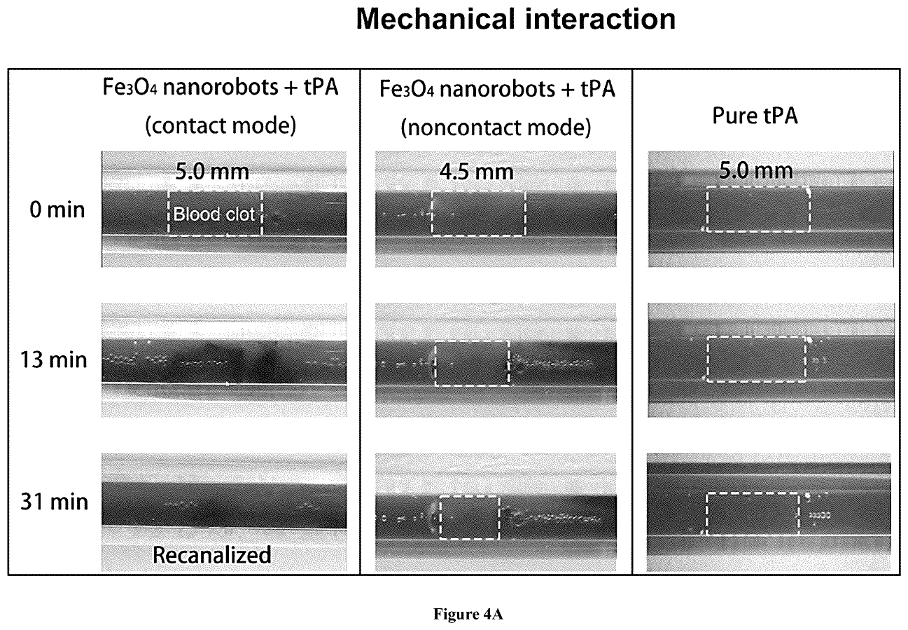

FIG. 3I: Adhesion of the tPA-nbots to the blood vessel wall after treatment. Left photograph showing one representative blood vessel branch after retrieval process. Right microscope images show the nanoparticle's adhesion on the blood vessel wall of different branches. For area a, it is the position near the catheter tip. And it involves the nanorobots release, accumulation, locomotion, and retrieval process; thus, nanorobots were more likely to contact with the wall, resulting in enhanced adhesion. For other distal branches, no obvious nanorobots adhesion was observed. In the dynamic blood environment, the nanorobots show no serious adhesion to the blood vessel wall.

FIGS. 32A to 32D: Thrombolysis rate and retrieval percentage of tPA-nbots in the blood vessel with various diameters. (A) The photo shows the blood vessel distribution of the human placenta and selected four regions with various diameters. (B) The thrombolysis process for four different regions of blood vessels with diameters ranging from 1.5 mm to 4.0 mm using tPA-nbots (2 mg). (C) Thrombolysis rate of using tPA-nbots (2 mg) blood vessel with various diameters (1.5 mm, 2.5 mm, 3.1 mm, and 4.0 mm). (D) Retrieval rate of tPA-nbots in the in the blood vessel with various diameters (1.5 mm, 2.5 mm, 3.1 mm, and 4.0 mm). Experimental conditions were set as: Injection speed: 0.05 mL/s, Concentration: 2 mg/mL, Dose: 2 mg, Magnetic field strength for trapping and locomotion: 64.9 mT, Flow speed: 2 cm/s).

FIGS. 33A to 33G: In vivo demonstration of the trapping capability of tPA-nbots during both occlusion and post-occlusion phases. (A) Construction of a controllable occlusion model at the carotid artery with an inner diameter of 0.9 mm in an 8-week New Zealand white rabbit. (B) Administration of tPA-modified nanorobots via catheter, followed by their confinement through utilization of a rotating magnetic field. (C) Controllable locomotion of nanorobots towards the occlusion site. The tPA-nbots were actuated with an external magnetic field (strength: 64.9 mT, frequency: 3 Hz). (D) Removal of hemostatic clip to recover the blood flow. (E) Magnetic propulsion of tPA-nbots towards the catheter. Upon reaching the catheter's terminal point, the nanorobots were drawn into the catheter, and subsequently, the catheter was extracted from the vessel, completing the vascular procedure. (F) Comparison of the carotid artery before and after retrieval of the tPA-nbots. (G) Iohexol injection for enhanced visualization of the vessels, allowing clear observation of the recanalization.

FIGS. 34A to 34C: In vivo safety analysis of the tPA-labeled microswarms intravenous injection. (A) Comprehensive blood analysis panel of mice injected with saline (control), mice injected with tPA-nbots (5 mg/mL, 10 mg/mL and 25 mg/mL) (n=3). Blood samples were collected on day 4. (B) H&E-stained histological sections of major organs, including the heart, liver, spleen, lung and kidney, from nontreated mice and mice treated with the tPA-Labeled microswarm (10 mg kg-1). Scale bar, 250 μm. (C) In vivo biodistribution of Fe over 1 day and 1 week in rats. Aliquots of the tPA-labeld Fe₃O₄ nanorobots were intravenously injected into the rats. The iron concentrations in the organs were determined at different time points after injection using ICP-MS. Error bars indicate the SD for n≥3.

FIG. 35: Comprehensive blood analysis panel of mice injected with saline (control), mice injected with tPA-nbots (5 mg/mL, 10 mg/mL and 25 mg/mL) (n=3). Blood samples were collected on day 15.

### DETAILED DESCRIPTION OF THE INVENTION

This invention provides a nanobot for recanalization of a bodily tube. In one embodiment, said nanorobot comprises: a) a magnetic core; b) a mesoporous shell coated on said magnetic core; and c) an outer layer of a chemical for recanalization bound to said mesoporous shell; wherein said nanobot is adapted to respond to an external magnetic field to carry out motion for mechanical and chemical recanalization.

In one embodiment, said chemical for recanalization comprises thrombolytic agents.

In one embodiment, said chemical for recanalization comprises tissue plasminogen activator.

In one embodiment, said magnetic core comprises one or more of Fe, Co, Ni, Mn, and their oxides or alloys.

In one embodiment, said magnetic core comprises Fe₃O₄.

In one embodiment, said mesoporous shell is made of a material comprising metal-organic framework (MOF), or cell membrane.

In one embodiment, said mesoporous shell comprises silica, Zeolitic Imidazolate Framework-8, Red Blood Cell Membrane or polydopamine.

In one embodiment, said external magnetic field comprises one or more selected from the group consisting of static magnetic field, tumbling magnetic field, rotating magnetic field, oscillating magnetic field, conical magnetic field, and gradient magnetic field.

In one embodiment, said motion comprises staying stationary, rotating, or moving along a desired trajectory.

In one embodiment, said nanobot has a size range of 20-5000 nm.

In one embodiment, said nanobot has a size of about 300 nm.

This invention also provides a system for recanalization of a bodily tube. In one embodiment, said system comprises: a) a plurality of the nanobots of this invention; b) a delivery mechanism for delivering said plurality of nanobots to said bodily tube; and c) a magnetic source adapted to provide an external magnetic field for controlling motion of said plurality of nanobots.

In one embodiment, said delivery mechanism is a catheter comprising a tip adapted to store and release said plurality of nanobots.

In one embodiment, said external magnetic field comprises one or more selected from the group consisting of static magnetic field, tumbling magnetic field, rotating magnetic field, oscillating magnetic field, conical magnetic field, and gradient magnetic field.

In one embodiment, said system further comprises a flow-reducing mechanism for blocking or reducing the flow rate to said bodily tube.

This invention further provides a method for recanalization of a bodily tube using the system of this invention. In one embodiment, said method comprises the steps of: a) Introducing said plurality of nanobots to vicinity of a recanalization site within said bodily tube using said delivery mechanism; b) Directing said plurality of nanobots to contact said recanalization site using from said magnetic source; and; c) Allowing said plurality of nanobots to mechanically interact with said recanalization site in a rotating magnetic field from said magnetic source.

In one embodiment, said system further comprises the step of retrieving said plurality of nanobots after step (c).

In one embodiment, further comprising the step of reducing flow or blocking said bodily tube prior to introducing said plurality of nanobots in step (a).

In one embodiment, said bodily tube comprises one or more selected from the group consisting of blood vessels, vas deferens, and gland ducts.

### System Design

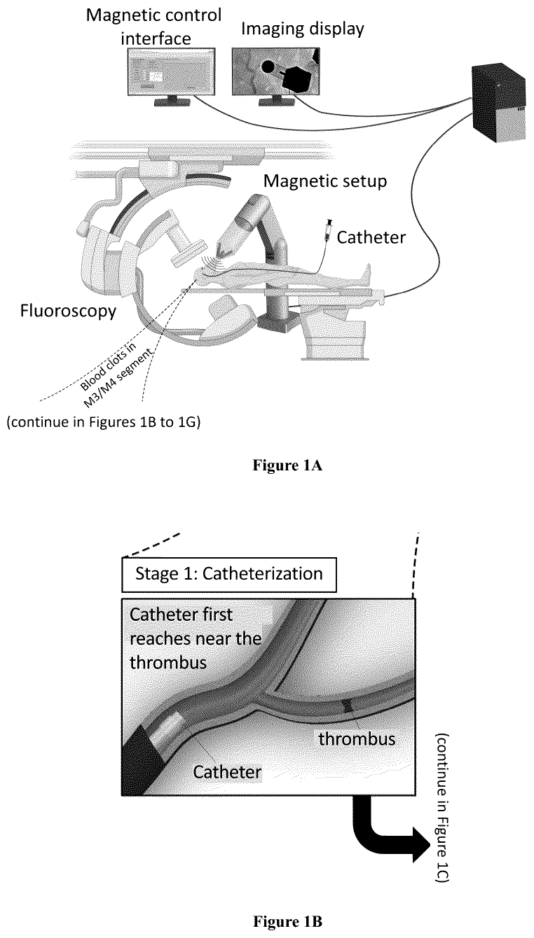

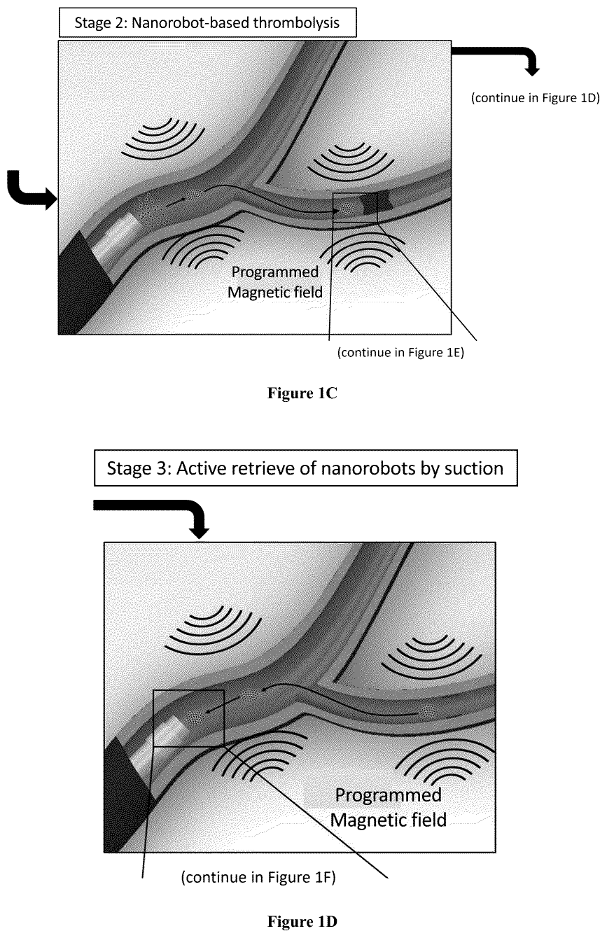

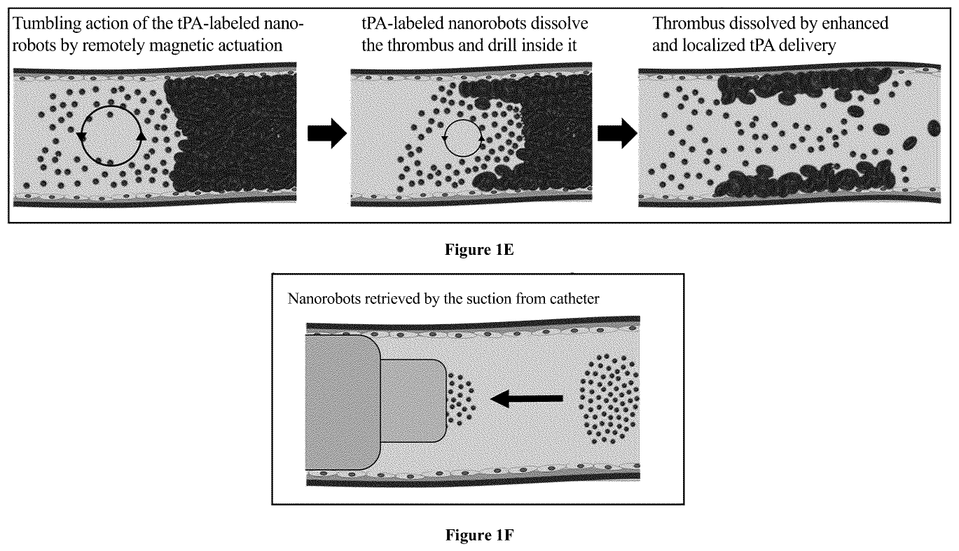

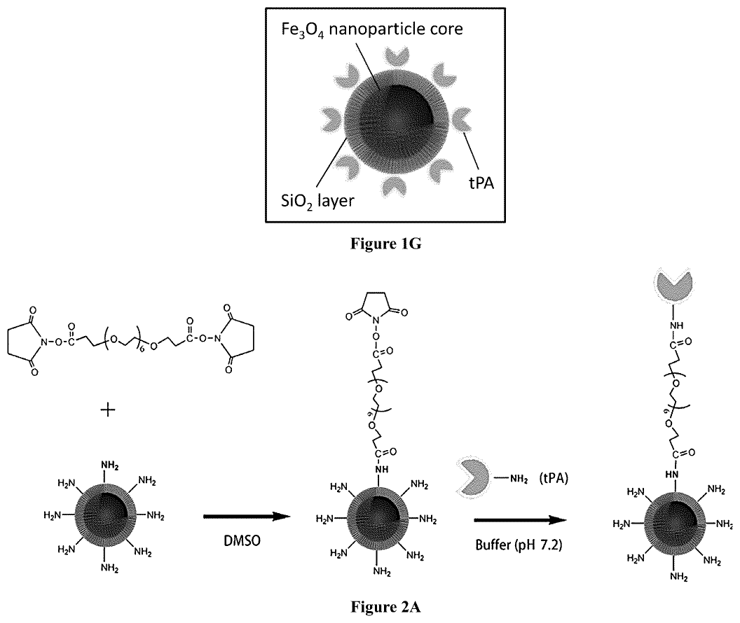

In general, blood vessels in the human body consist of numerous branches. In addition, the blood vessels exhibit varying diameters based on the degree of fractalization. In the case that a blood clot appears in the M1/M2 designated blood vessels, where the catheter is accessible, the blood clot can be treated by the catheter-based intervention (with distal-tip balloons for protection during the thrombus retrieval). Afterward, some residual (small) fragments of the thrombus are able to enter M3/M4 segments and distal with the smaller diameter; thus, another thrombus may be formed. Similarly, the blood clot may appear in the M3/M4-designated blood vessels to begin with. In this scenario, achieving spatially highly controlled treatment is challenging, often necessitating systemic treatment with tPA and its serious side effects. To avert this systemic treatment and to achieve imaging-guided delivery of retrievable tPA-nbots to the blood clot for thrombolysis, a system comprising a catheter, fluoroscopy, and mechanical arm-equipped magnetic control unit for tPA-nbots actuation was developed, as illustrated in FIGS. 1A to 1G. The advantages of the system compared to previous work are that it can precisely deliver the nanorobots for thrombolysis and can collect (remove) the nanorobots from the blood vessel after thrombolysis (FIGS. 1A to 1G). The thrombolysis process mainly comprises three steps, as schematically shown in FIGS. 1A to 1G. First, the catheter loaded with the tPA-nbots is pushed to the vicinity of the fork between the blood vessels of M1/M2 and M3/M4 segments. Second, the nanorobots are released from the catheter and navigated by an external magnetic field toward the site of blood clot in the M3/M4 segments. Successful guiding of the tPA-nbots is followed by fluoroscopic imaging. Then, under the tumbling motion-triggered rotation and translation of the nanorobots, the surface of the individual nanorobots gets in contact with the clot, initiating the contact-induced thrombolysis process until the clot is completely lysed. The precisely controlled magnetic field and controlled hydrodynamic flow contribute to the highly efficient dissolution of the blood clots by this process. Third, the tPA-nbots are steered back to the tip of the catheter to gather and remove them from the blood vessels (retrievability), reducing the side effects of the tPA-nbots considerably.

Fabrication of Fe₃O₄ωmSiO₂-tPA Nanorobots

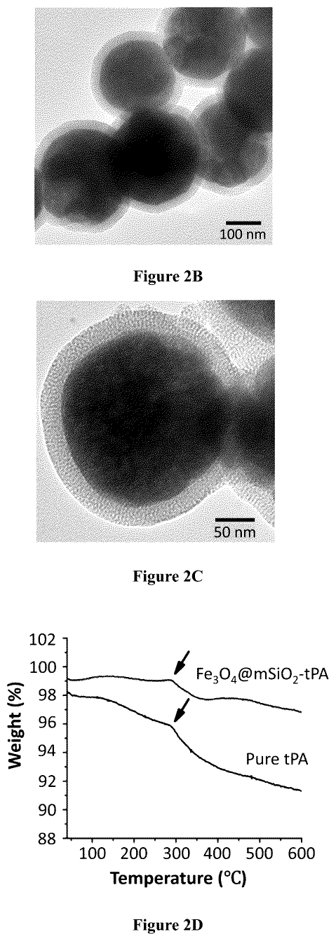

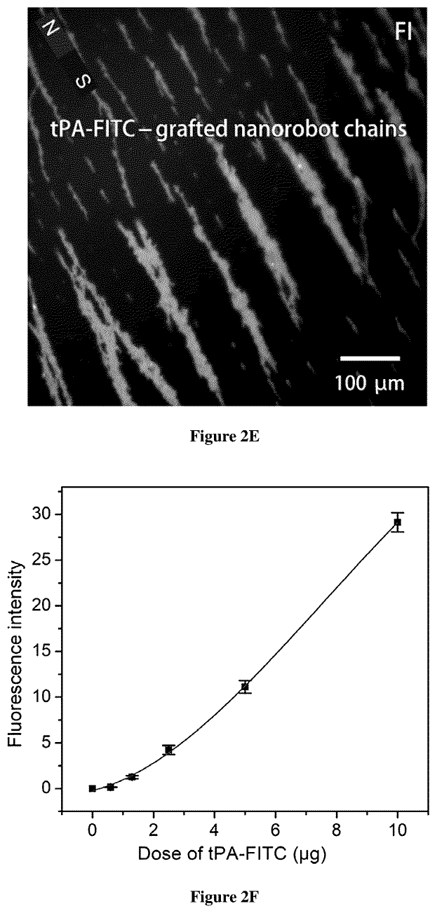

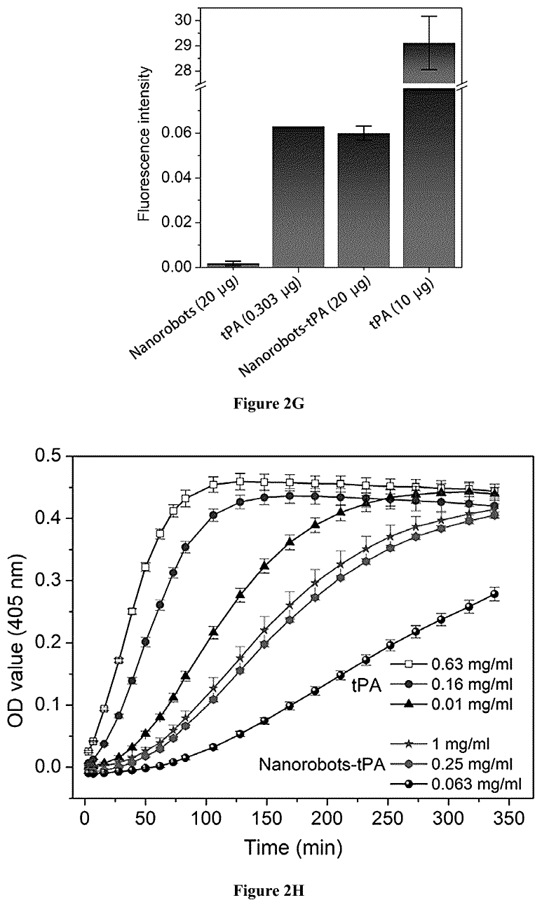

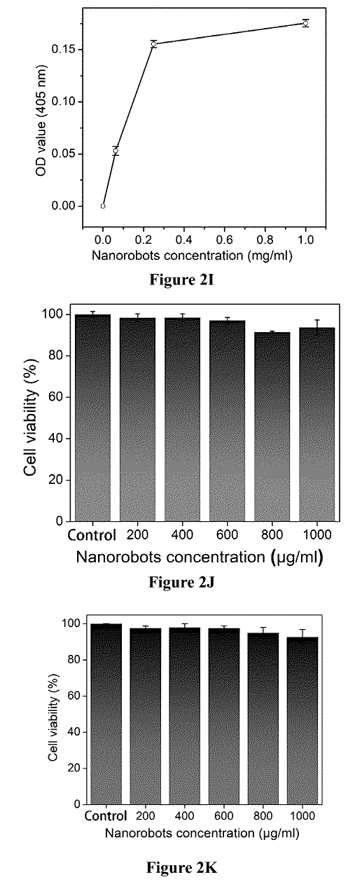

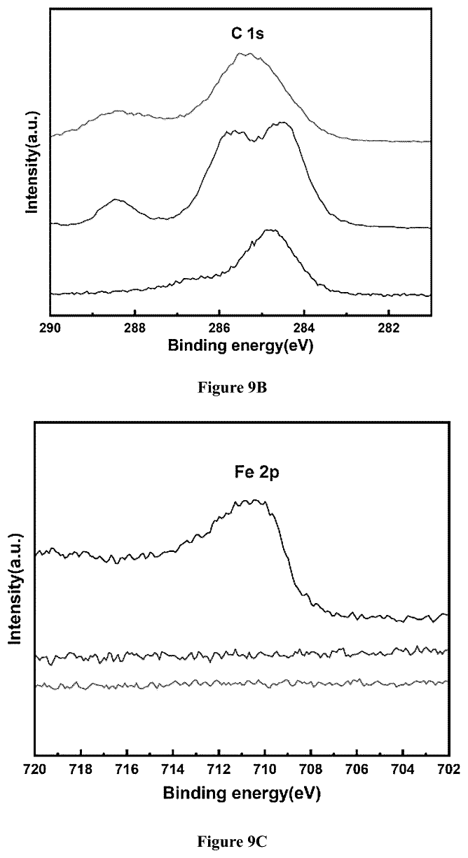

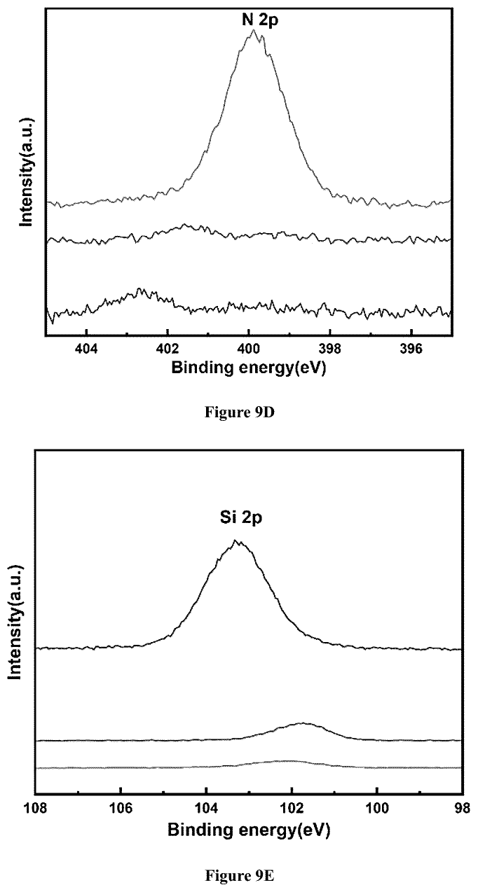

tPA-nbots composed of a magnetic core, a mesoporous silica shell, and a grafted tPA outer layer (robot size is ˜300 nm) were fabricated in four steps (FIGS. 2A to 2K). The tPA molecules are chemically bonded onto the nanorobots surface to maximally increase the local enrichment and accumulation of the drug, therefore enabling the retrieval of the tPA after the thrombolysis treatment and reducing the drug dose. First, the fabrication of the magnetic cores was conducted with a solvothermal method that used ethylene glycol (EG) as the solvent and reduction agent and polyethylene glycol (PEG) as the surfactant. Second, a silica shell was coated onto the magnetic core via a biphasic stratification approach, using cetrimonium chloride (CTAC) as the surfactant and template, water as the solvent for dispersion of the magnetic spheres, and octadecene as the solvent of the silicon precursor [tetraethyl orthosilicate (TEOS)]. This process resulted in interfacial hydrolysis of the silicon precursor, building up the silica shell on the magnetic core. Third, surface modification processes were applied to successively graft the —NH₂ terminated 3-aminopropyltricthoxysilane (APTS) and the bis-N-hydroxysuccinimide (NHS)-PEG6 on the surface of the Fe₃O₄ωmSiO₂ spheres to endow the surface with soft and biocompatible PEG tails. Furthermore, the hydroxylsuccinimide moiety yields a site for coupling proteins. Last, the protein tPA was chemically bound to the surface of the Fe₃O₄ωmSiO₂ spheres, resulting in the tPA-nbots. Here, the protein binds at room temperature and mild pH via a peptide bond to the carbonyl, while the succinimide is the leaving group. The anchoring of the tPA on the Fe₃O₄ωmSiO₂ spheres is of utmost importance for the enhancement of thrombolysis. The as-prepared tPA-nbots comprise a core-shell structure with lots of mesopores in the silicon shell, as shown in FIGS. 2B and 2C. Fourier transform infrared spectroscopy (FTIR) and x-ray photoelectron spectroscopy (XPS) were used to scrutinize and confirm the surface coating. The XPS spectra of the Fe₃O₄ωmSiO₂ composite revealed the presence of both Fe and Si elements (FIGS. 9A to 9E).

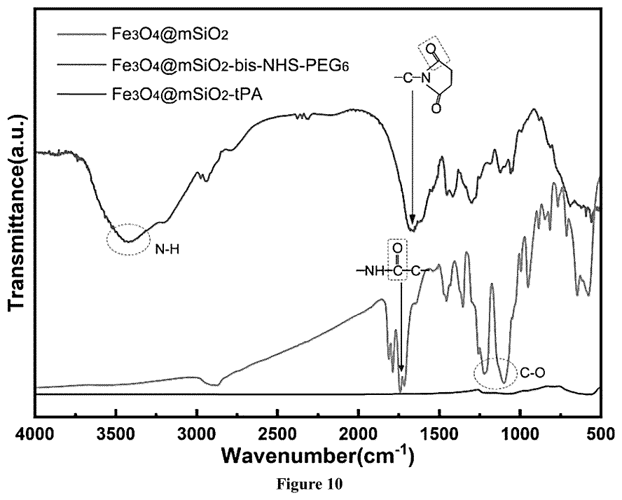

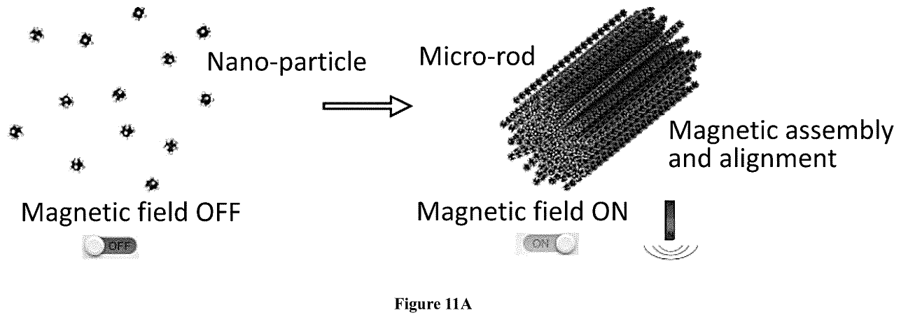

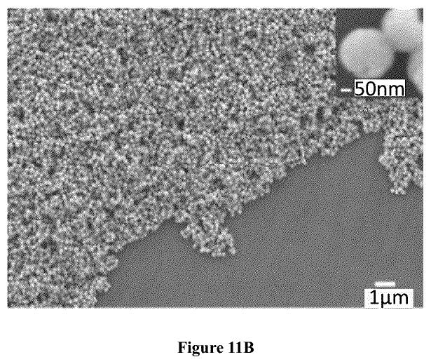

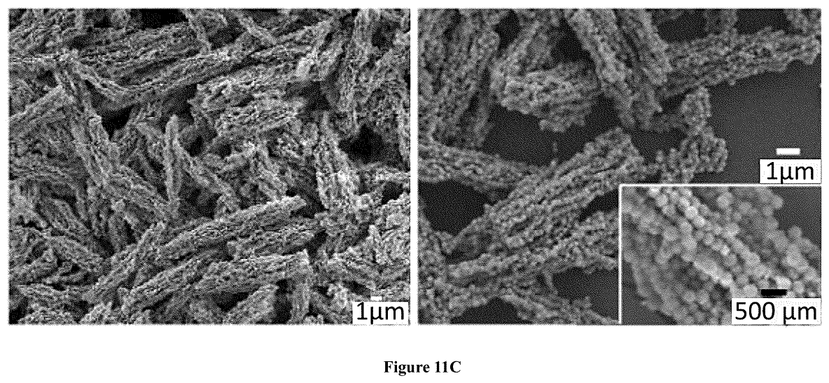

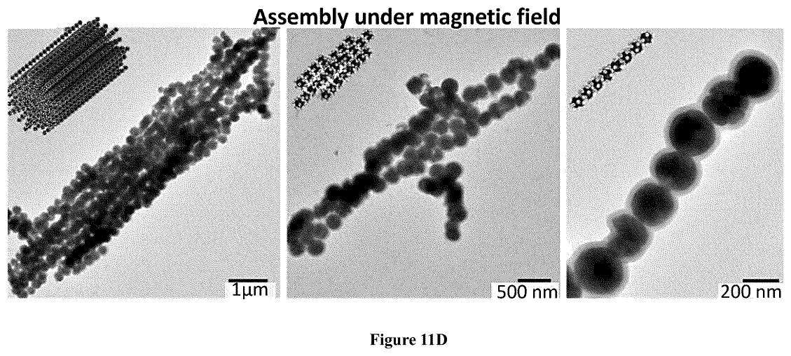

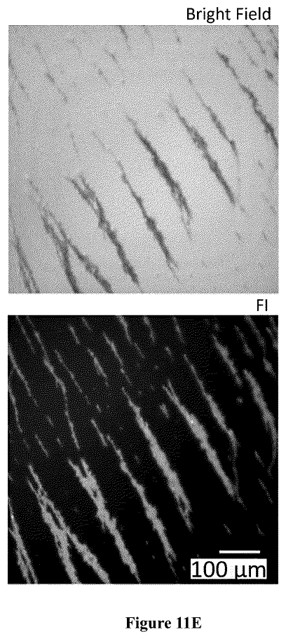

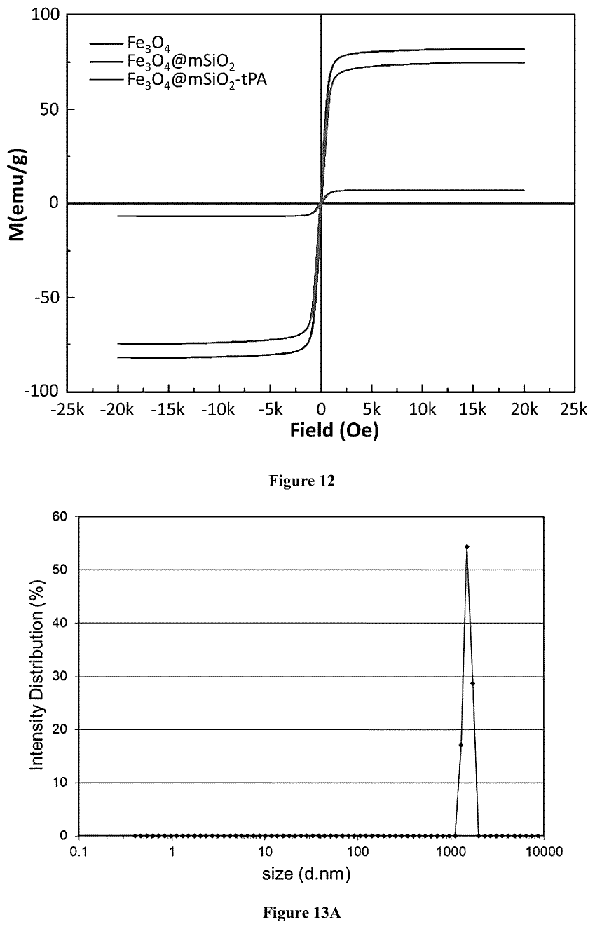

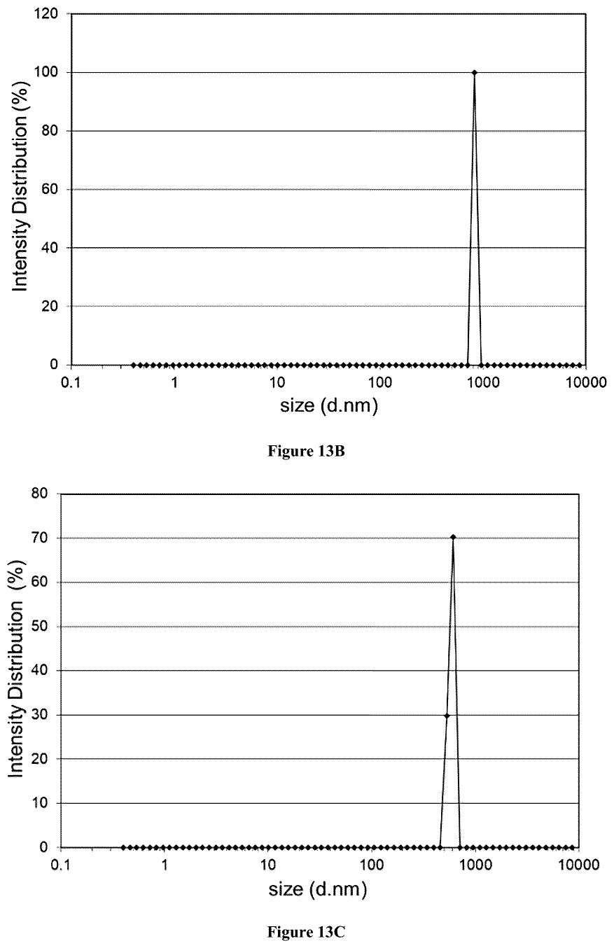

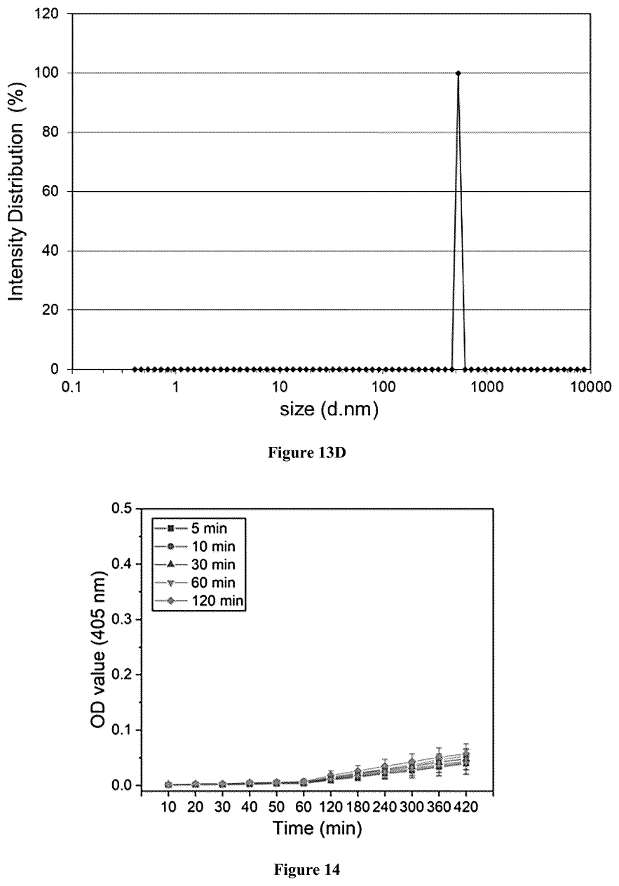

Subsequent surface coating with bis-NHS-PEG6 resulted in a decrease in the peaks corresponding to Fe 2p and Si 2p. Consequently, the coating impedes the accurate detection of the underlying Fe₃O₄ and mSiO₂ components. Following the anchoring of tPA, the emergence of the N Is peak confirmed the successful coating of tPA molecules onto the composite surface. The surface functionalization of bis-NHS-PEG6 and anchoring of tPA were also inspected by FTIR (FIG. 10). The appearance of C—O on succinimide group signals at ˜1750 to 1730 cm I confirms the successful grafting of bis-NHS-PEG6 on the Fe₃O₄ωmSiO₂ composite. The attenuation of the C—O on succinimide group signals at ˜1750 to 1730 cm⁻¹ and the appearance of C—O on peptide bond signals at around 1780 to 1750 cm⁻¹ denote the successful anchoring of tPA, forming the Fe₃O₄ωmSiO₂-tPA nanorobots (tPA-nbots). The thermogravimetric analysis curves of the pure tPA, and tPA-nbots are depicted in FIG. 2D, indicating that the tPA-nbots contain tPA, as the tPA starts to decompose at around 300° C. To evaluate the grafting amount of tPA on the Fe₃O₄ωmSiO₂ spheres, tPA were labelled with fluorescein isothiocyanate (tPA-FITC) and the fluorescence intensity was measured by a plate reader. Fluorescent and magnetic steerable tPA-FITC-nbots were visualized by fluorescence microscopy, as shown in FIG. 2E. While a static magnetic field was applied, the tPA-nbots inside the solution oriented and aligned to form lots of microscale particle chains, as shown in FIGS. 11A to 11E. The length and thickness of the chains vary in hundreds of micrometers, as indicated by the transmission electron microscopy images in FIGS. 9A to 9E. The stability of these chain bundles is dynamic and inherently transient. The tPA-nbots are superparamagnetic with their remanence being approximately 0 (FIG. 12). That is, while the external magnetic field is removed, the chain bundles will easily disassemble into the individual nanoparticles because the interactive dipole attractive forces between the tPA-nbots are disappeared. FIGS. 13A to 13D illustrates measurements of tPA-nbot diameter under magnetic actuation (64.9 mT, 3 Hz) in a simulated vessel environment with a flow speed of 5 cm/s. Following the removal of magnetic field, the tPA-nbot chains collapsed and the diameter decreased to 573 nm within 30 min. These findings highlight the dynamic structural changes of tPA-nbot chains, emphasizing the facile disassembly of initially formed chain bundles in response to circulatory conditions after magnetic influence removal.

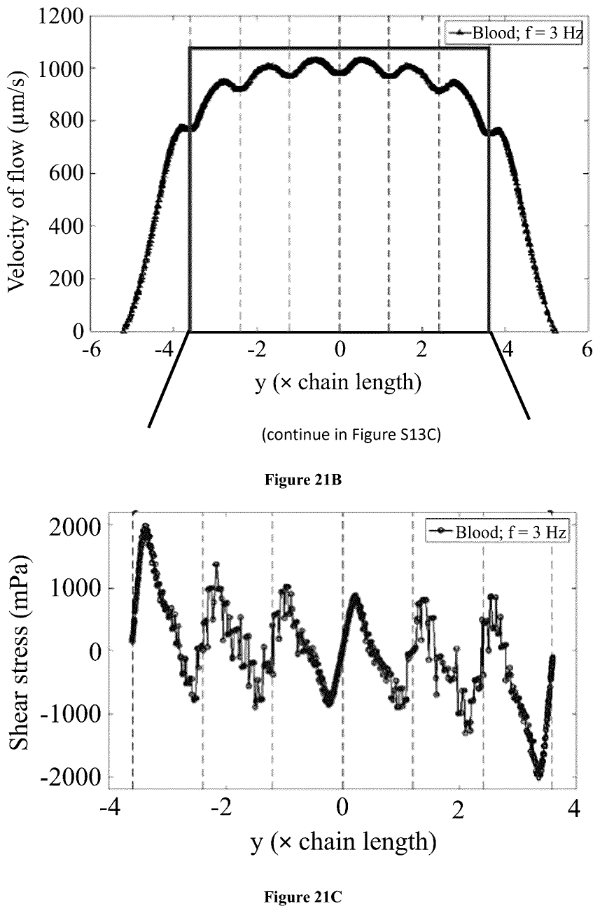

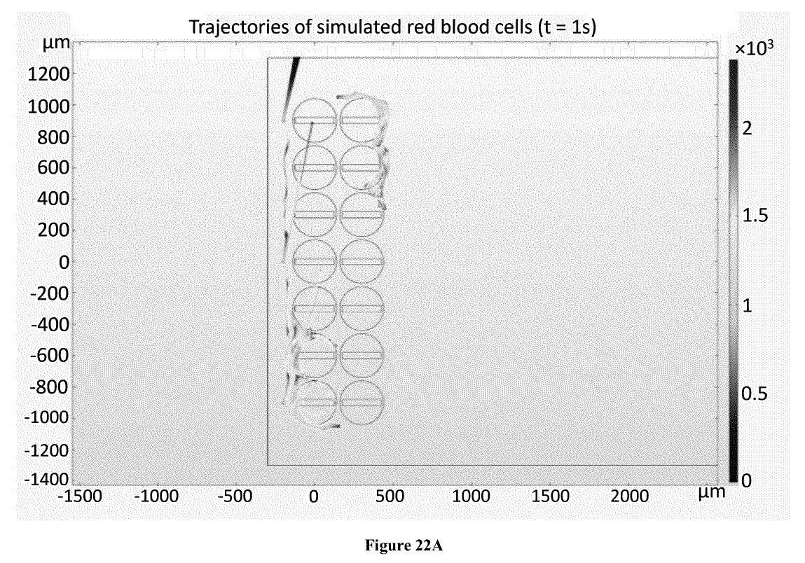

The standard relation curve between the fluorescence intensity and the dose of the tPA-FITC was measured and was plotted in FIG. 2F. After comparing the fluorescence intensity of the Fe₃O₄ωmSiO₂ spheres before and after the tPA-FITC anchoring process and the standard curve, the anchoring capacity of the tPA on the Fe₃O₄ωmSiO₂ was calculated to be about 15 mg/g. The enzymatic activity of the anchored tPA on the tPA-nbots was assessed with the tPA Human Chromogenic Activity Assay Kit by measurement of the course of the optical density (OD) value at 405 nm dependent on the reaction time. The PLG reacts with the tPA and is used as a colorimetric assay for evaluating the activity of the anchored tPA. As indicated in FIGS. 2G and 2H, the tPA-nbots show a similar trend of increasing in absorptance at 405 nm, demonstrating the enzymatic activity of the surface tPA. From the curves, it was acquired by calculation that the tPA-nbots show compatible enzymatic catalysis with the pure tPA that contains the same amount of tPA, denoting that the activity of the surface tPA is well preserved without deactivation. The number of tPA molecules on a single tPA-nbot is calculated to be about 9450. During the swarming motion of nanorobots, a small fraction of initially anchored tPA may detach, as evidenced by FIG. 14; however, the majority of tPA molecules remain strongly bonded to the nanorobotic surface, as indicated by measuring the OD value (405 nm) of residual solution subsequent to the removal of tPA-nbots, even after prolonged swarming times. FIG. 21 shows the OD value after 128 min of enzymatic catalysis for different concentrations of tPA-nbots. The OD value increases with the increase of the tPA-nbot concentration up to a concentration 0.25 mg/ml. At higher concentrations, the increase in OD is shallow and may be due to saturation and diffusion effects. The cell viability of mesenchymal stem cells (FIG. 2J) and human umbilical vein endothelial cells (FIG. 2K) verified the high biocompatibility of the tPA-nbots.

Motion Control of the tPA-Nbots Microswarm in Vessel

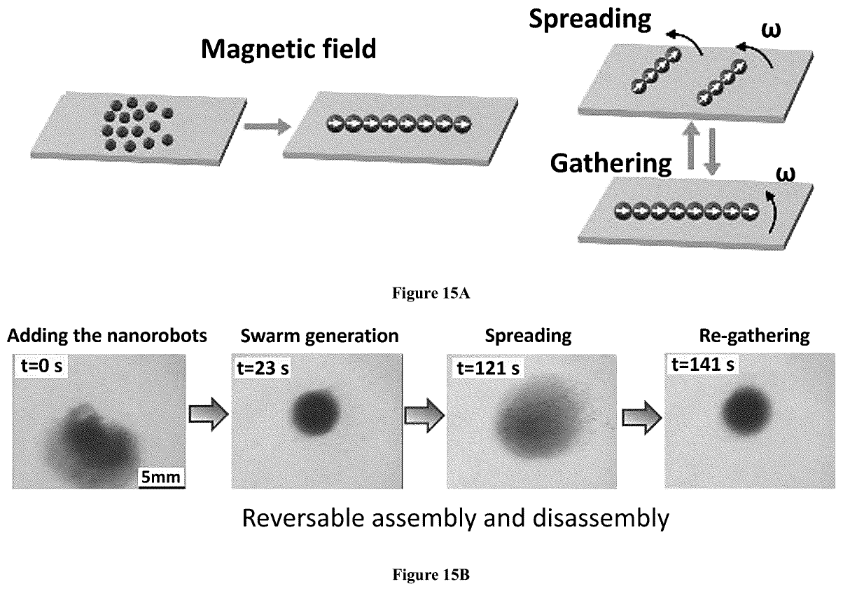

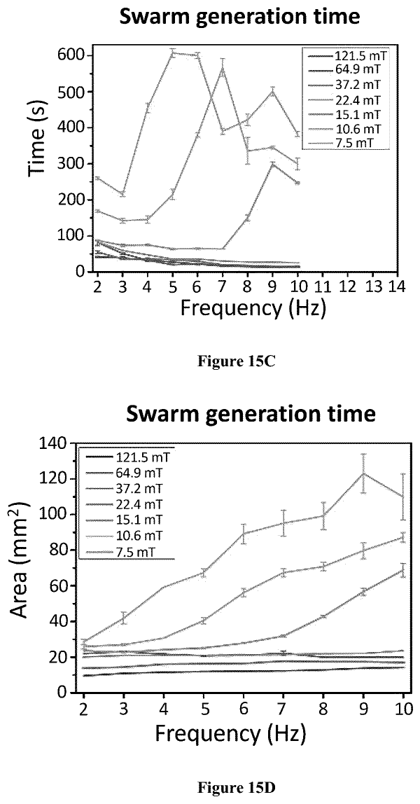

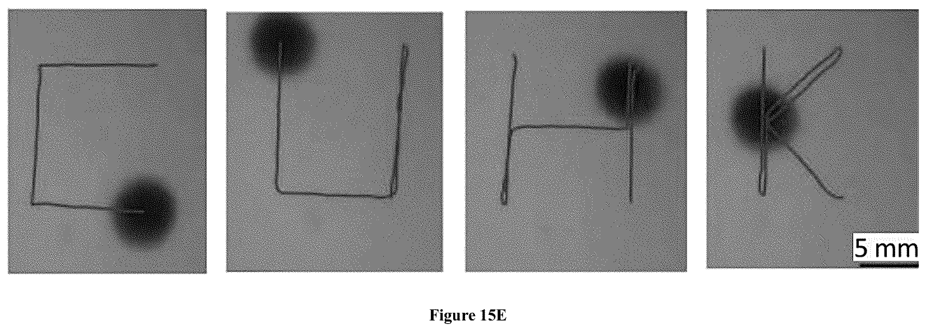

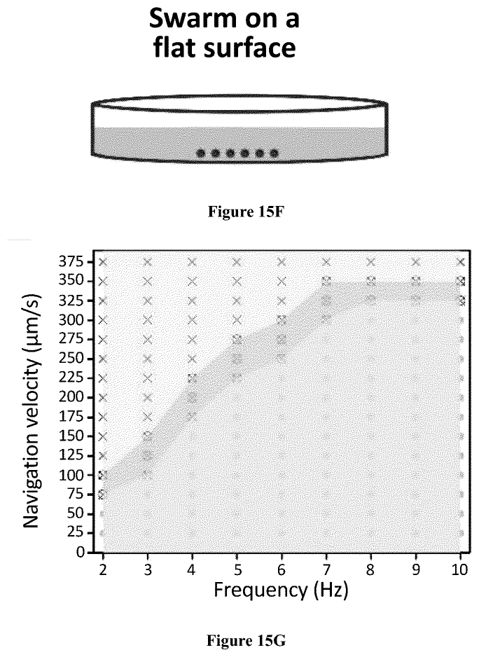

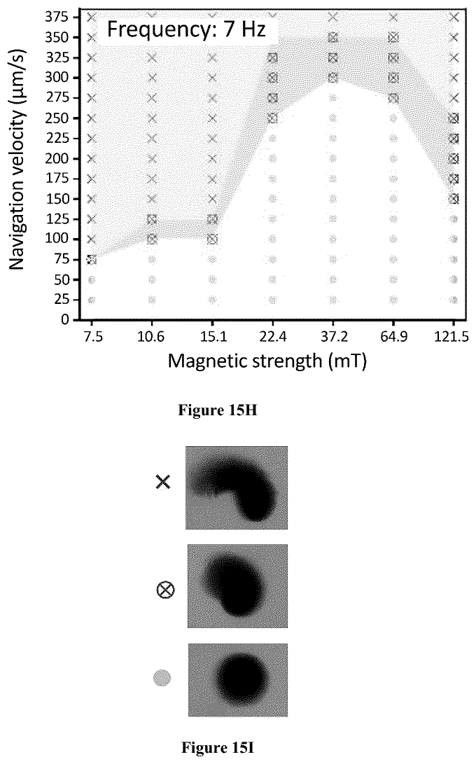

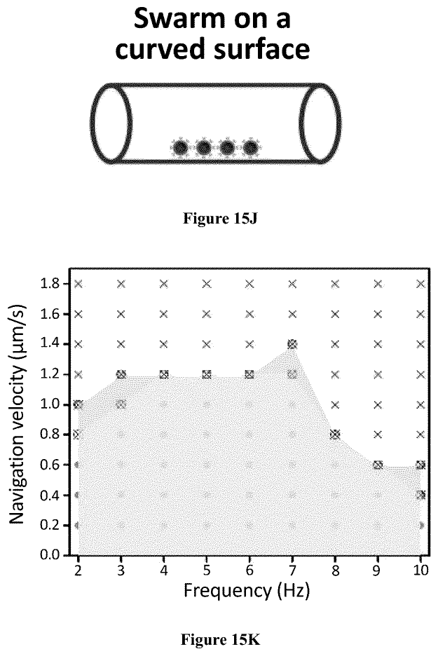

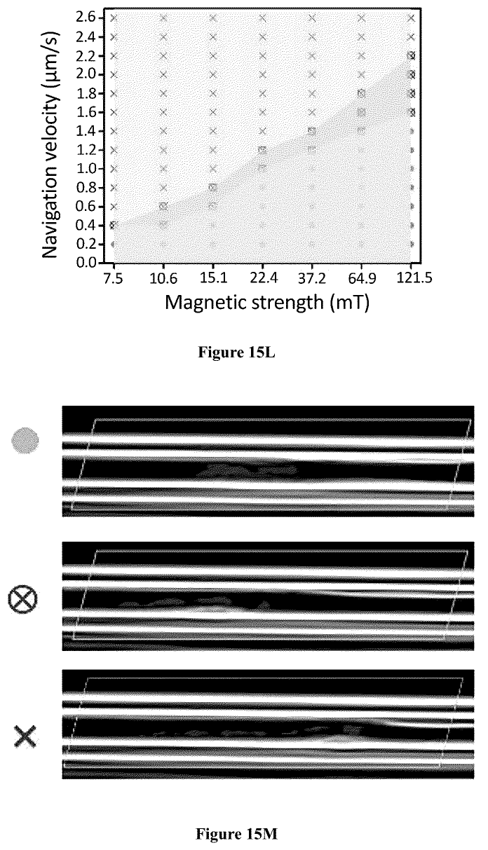

The directed locomotion of tPA-nbots inside the vessel is generally the swarming motion on a curved surface along the axis direction. The swarming motion of the tPA-nbots is triggered by a rotating magnetic field (RMF) in the experimental plane (39-41). Actuated by the external magnetic fields, adjacent magnetized nanorobots form chains because of the dipole attractive forces (FIG. 15A). The interactive magnetic force of a couple of nanorobot chains (Fᵢ) can be expressed as (42)

$${F}_{i} = \frac{3 {μ}_{0} {m}_{i} {m}_{j}}{4 π {r}_{i , j}^{4}} \{ [ 1 - 5 {( m \cdot {r}_{i . j} )}^{2} ] {r}_{i . j} + 2 ( m \cdot {r}_{i , j} ) m \} \qquad Formula ( 1 )$$

where mᵢ and mᵢ represent the magnetic moment norms of the adjacent nanorobot chains whose direction is m. The direction of m can be controlled to generate either attractive or repulsive forces between dissimilar chains, resulting in the assembly (gathering) and disassembly (spreading) of the colloidal microswarm, as shown by the optical images in FIG. 15B.

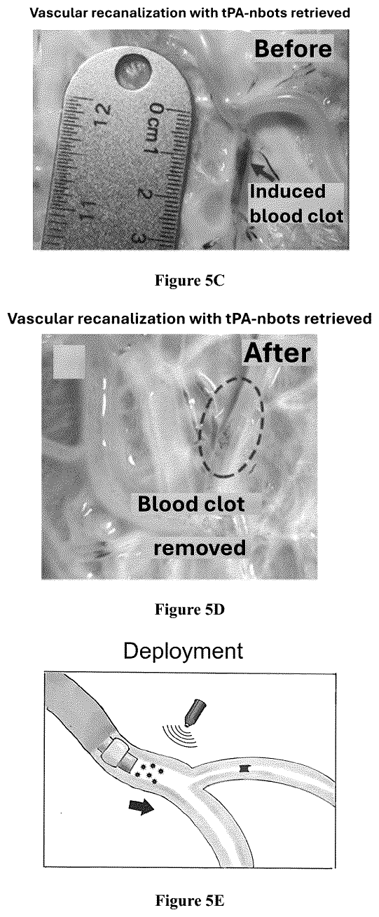

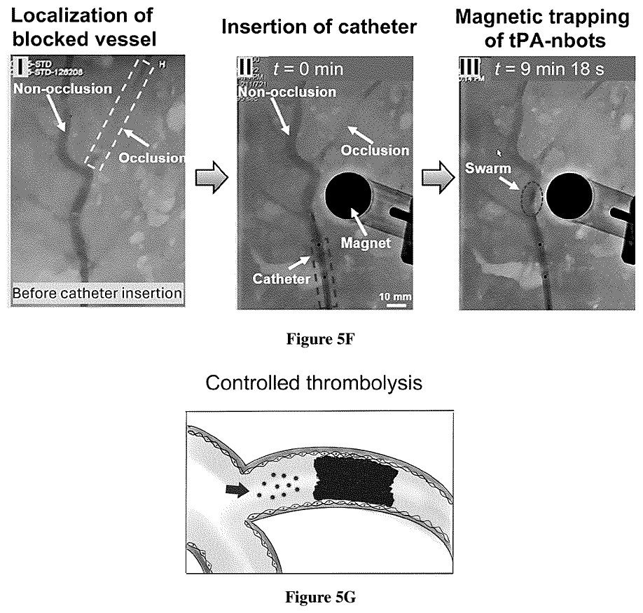

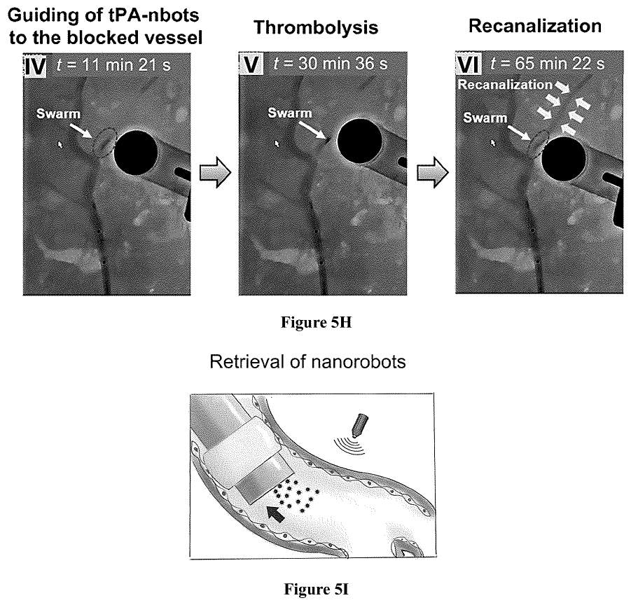

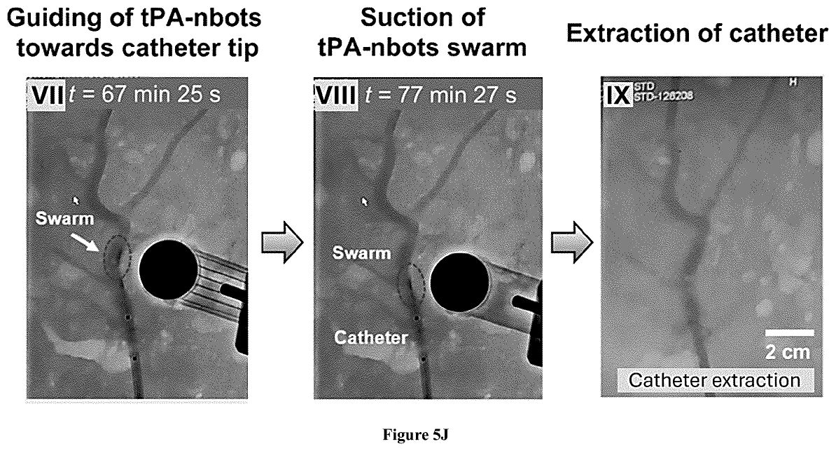

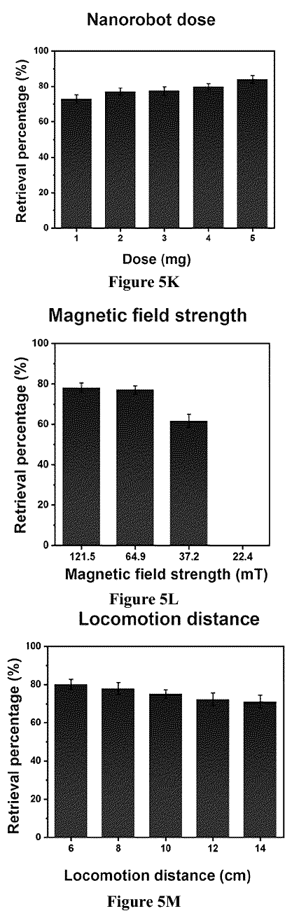

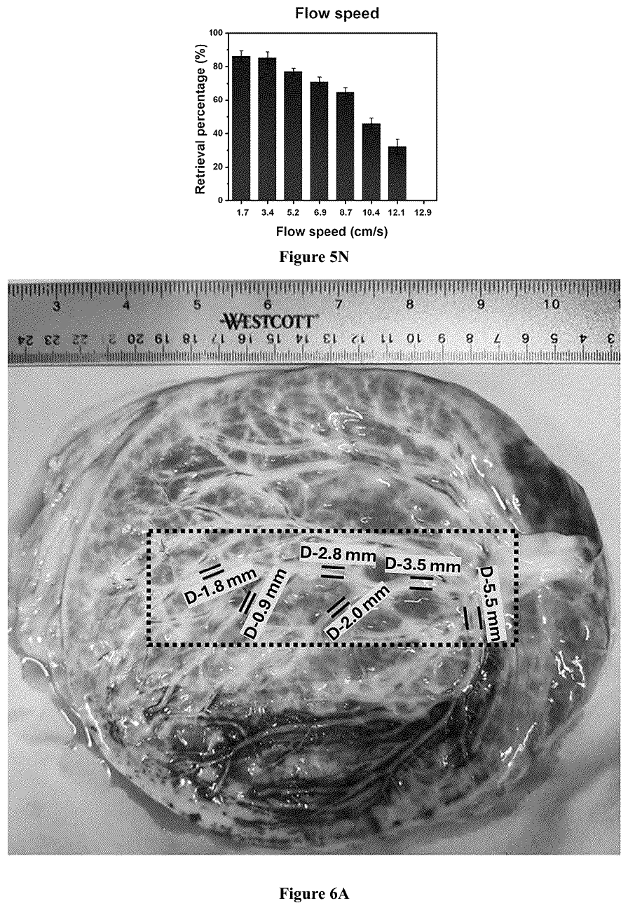

The generation time of the stable colloidal microswarm, as well as the generation area, can be controlled by adjusting the input frequency as illustrated in FIGS. 15C and 15D. The as-generated colloidal microswarm can perform on-demand locomotion along predesigned trajectories (FIG. 15E and movie S1: Colloidal microswarm generation and motion control on a plane or on curved tube by using magnetic field control). The locomotion on both flat surface and curved surface was quantitatively investigated in FIGS. 15F to 15M. The phase diagrams in FIGS. 15G to 5I show that the increase of the locomotion speed will cause instability of the colloidal microswarm, resulting in partial lagging of the colloidal microswarm. The maximum locomotion speed of a stable colloidal microswarm reaches up to ˜300 μm/s at 37.2 mT on a flat surface, and it decreases once a magnetic field strength exceeds 64.9 mT. For swarming motion inside the vessel, the stable patterns (FIG. 15K to 15M) are easily generated at a relatively low swarming motion speed. The maximum locomotion speed of a stable nanorobot swarming pattern increases with the input frequency up to a frequency of 7 Hz. At frequencies exceeding 7 Hz, the maximum velocity dwindles sharply. Furthermore, the maximum locomotion speed increases monotonically with the field strength and a maximum velocity of around 1.4 mm/s is observed at 121.5 mT.

### Retrievability of the Nanorobots

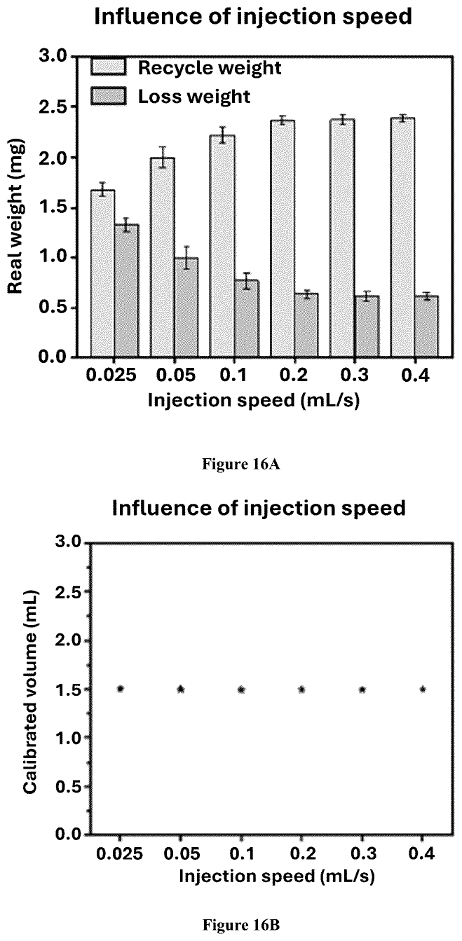

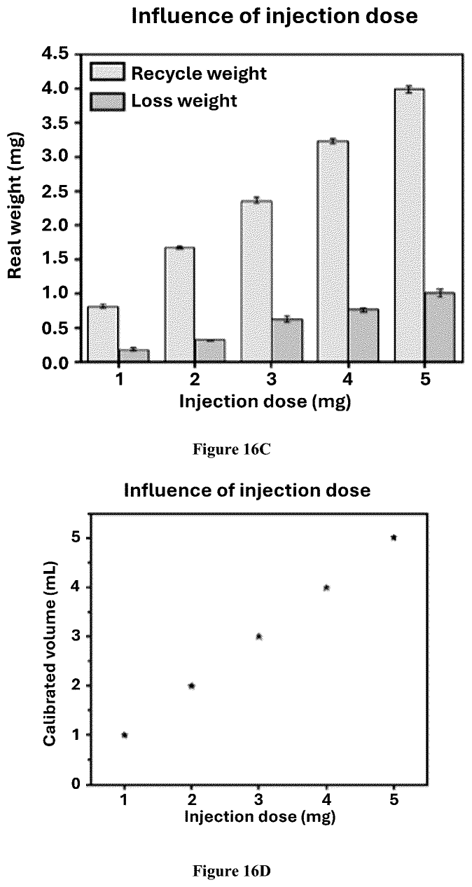

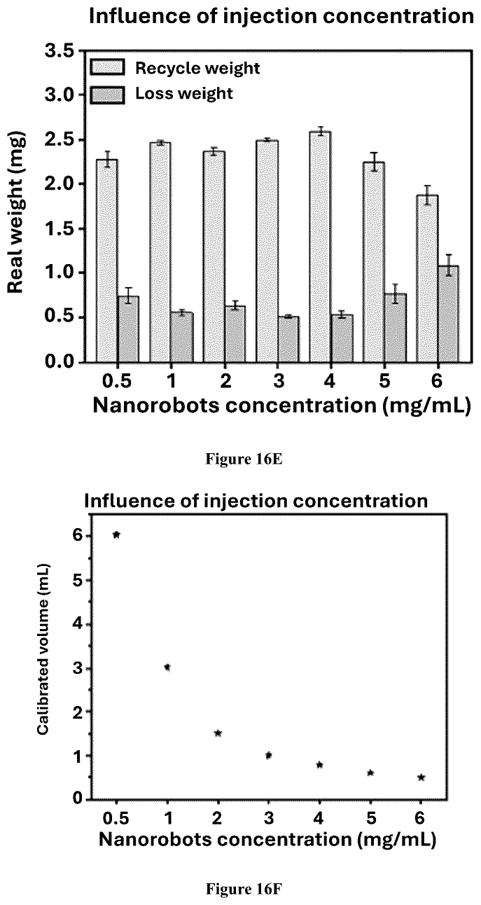

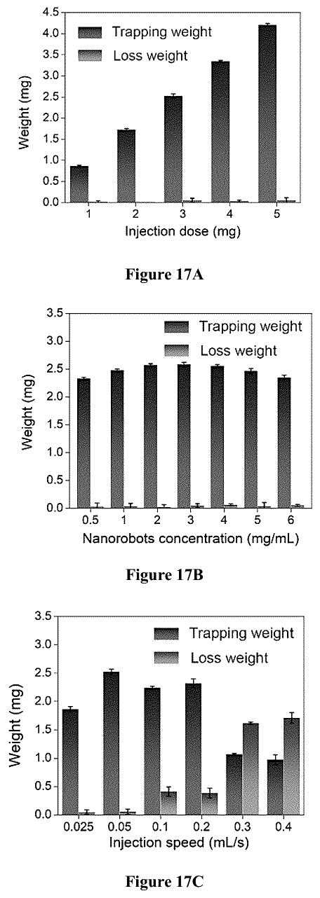

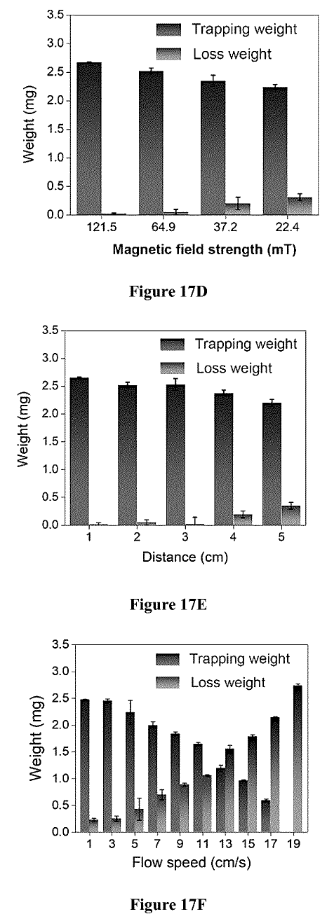

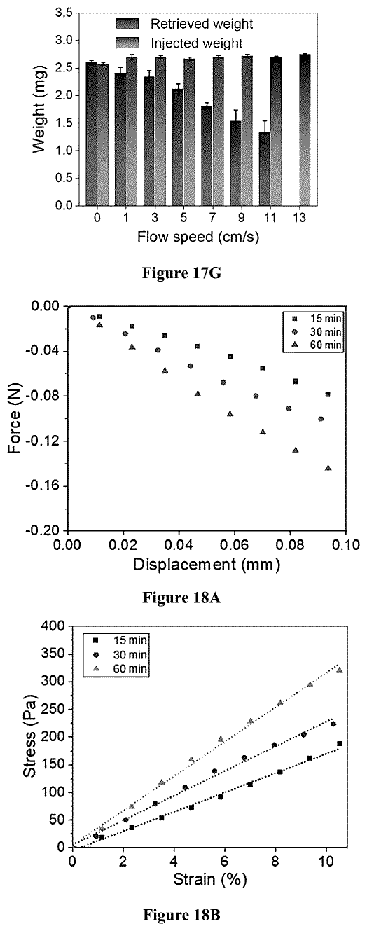

The retrievability of the tPA-nbot microswarm is also investigated, including two steps, i.e., directed delivery of the tPA-nbots to the targeted site for therapy (step 1) and swimming back of the tPA-nbots toward the catheter (step 2). Step 1 is a downstream process, whereas step 2 is an upstream process with larger resistance. In the present system, the tPA-nbots inside the vessel are retrievable by an integrated system composed of a catheter and a magnetic actuation setup (FIG. 3A). The tPA-nbots were injected by a syringe connected to the catheter to simulate a vessel with various nanorobot concentrations, injection doses, and injection speeds. The pulsatile flow inside the vessel was controlled with a pump. Furthermore, the magnetic field strength and the distance (namely, the distance between the catheter tip and the magnet) were controlled. The retrievable efficiency dependent on all the abovementioned factors was investigated (nanorobot loss inside the catheter, before entering the vessel is investigated in FIGS. 16A to 16F). (i) Injection dose: Both the trapping weight and the loss weight of the nanorobots increase linearly with the injection dose (FIG. 17A) because of the loss of nanorobots in the catheter. However, the trapping percentage of the nanorobots always remains larger than 95% with different injection doses [FIG. 3B; injection speed, 0.05 ml/s; dose, 3 mg; concentration, 2 mg/ml; diameter of saline tube, 2.5 mm (inner); length of catheter, 142 cm; diameter of catheter, 1 mm (inner); frequency, 3 Hz; field strength, 64.9 mT; distance between catheter tip and magnet center, 20 mm]. (ii) Nanorobot concentration: The trapping weight and the loss weight of the nanorobots show little variation (FIG. 17B), which shows a relatively larger value (˜2.6 mg) with the nanorobots concentration ranging from 2 to 4 mg/ml. The trapping percentage can be controlled at higher than 95% of the injected weight to the vessel with different nanorobot concentrations [FIG. 3C; injection speed, 0.05 ml/s; dose, 3 mg; diameter of saline tube, 2.5 mm (inner); length of catheter, 142 cm; diameter of catheter, 1 mm (inner); frequency, 3 Hz; field strength, 64.9 mT; distance between catheter tip and magnet center, 20 mm]. (iii) Injection speed: As shown in FIG. 17C, with the increase of the injection speed, the nanorobots loss during the catheter transportation decreases (FIGS. 16A to 16F), and the trapping capability by using a magnetic field becomes difficult due to the high transport velocity of nanorobots in the vessel. It shows the best trapping efficiency when the injection speed is 0.05 ml/s, with both high trapping percentage and low nanorobot loss in the catheter [FIG. 3D; dose, 3 mg; concentration, 2 mg/ml; diameter of saline tube, 2.5 mm (inner); length of catheter, 142 cm; diameter of catheter, 1 mm (inner); frequency, 3 Hz; field strength, 64.9 mT; distance between catheter tip and magnet center, 20 mm]. It is difficult to trap the nanorobots with an injection speed of more than 0.3 ml/s. (iv) Magnetic field strength: The nanorobots trapping efficiency decreases with the decrease of magnetic field strength [FIG. 3E and FIG. 17D; injection speed, 0.05 ml/s; dose, 3 mg; concentration, 2 mg/ml; diameter of saline tube, 2.5 mm (inner); length of catheter, 142 cm; diameter of catheter, 1 mm (inner); frequency, 3 Hz; distance between catheter tip and magnet center, 20 mm]. (v) Distance: The trapping weight slightly decreases with the increase of the distance (FIG. 17E), and the trapping percentage of the nanorobots remains up to 90% even when the distance reaches 5 cm [FIG. 3F; injection speed, 0.05 ml/s; dose, 3 mg; concentration, 2 mg/ml; diameter of saline tube, 2.5 mm (inner); length of catheter, 142 cm; diameter of catheter, 1 mm (inner); frequency, 3 Hz; magnetic strength, 64.9]. (vi) Simulated blood flow speed in the vessel: With the increase of the flow speed, the content of nanorobots trapped by the magnetic field decreases accordingly, and more than 74.1% nanorobots can be trapped even with a flow speed of 7 cm/s [FIG. 3G and FIG. 17F; injection speed, 0.05 ml/s; dose, 3 mg; concentration, 2 mg/ml; diameter of saline tube, 2.5 mm (inner); length of catheter, 142 cm; diameter of catheter, 1 mm (inner); frequency, 3 Hz; field strength, 64.9 mT; distance between catheter tip and magnet center, 20 mm]. While the flow speed exceeds ˜17 cm/s, nanorobots can hardly be trapped in swarming motion and overcome the flow by the applied magnetic field. A quantitative investigation of the retrieval efficiency under different flow speeds is performed. As shown in FIG. 3H and FIG. 17G, the retrieved weight of the nanorobots decreases with the increase of the flow speed due to the nanorobots loss (injection speed, 0.05 ml/s; concentration, 2 mg/ml; dose, 3 mg; field strength, 64.9 mT; locomotion distance, 12 cm; locomotion speed, 1 mm/s; extraction speed, 0.25 ml/s). To achieve a retrieval percentage higher than 80%, the flow speed should be slower than 5 cm/s.

Here, a theoretical model is proposed to investigate the forces and motion of tPA-nbots inside vessel, which is crucial for directed delivery and retrieval processes. To retrieve the nanorobots, the main difficulty is to overcome the fluidic resistance and friction under a low Reynolds number liquid (blood). In an aqueous environment without incoming flows, the tPA-nbots rotate on the plane with an angular velocity w and translate with a locomotion velocity v parallel to the plane under the action of external magnetic moment Tₘ. In the present case, the nanorobot is only subjected to the external magnetic moment Tₘ (without magnetic force) along its motion direction. A resistive torque (Tresistance) from the fluid should be overcome, which can be expressed as (43, 44)

$${T}_{resistance} = 8 π μ R 2 {T}^{t *} v + 8 π μ R 3 {T}^{r *} ω \qquad Formula ( 2 )$$

where T^{t}* and T^{r}* are the normalized scalar force and torque components.

$$Normalized scaler force : {T}^{t *} = - ( 1 / 10 ) ln ( δ / R ) + {O ( δ / R )}^{0} \qquad Formular ( 3 )$$

$$Normalized scaler torque : {T}^{r *} = - ( 2 / 5 ) ln ( δ / R ) + {O ( δ / R )}^{0} \qquad Formular ( 4 )$$

where the superscripts t and r represent the components of the translation and the rotation of the nanorobot, respectively; and δ is the gap between the plane and the bottom of the nanorobot.

In the presence of an external RMF, the external magnetic moment Texternal=V|M×B|=V|M|B |sinθ is exerted on the nanorobot to align its magnetization, where V is the nanorobot volume, M is the magnetization of the nanorobot, B is the magnetic field vector, and θ is the angle between M and B.

The uniform rotation of the nanorobot along the plane can be regarded as a series of equilibrium processes of external magnetic moment and the fluid resistance torque, that is, Tₑₓₜₑᵣₙₐₗ=T_{resistance}. Normally, at a certain magnetic rotating field, the magnetic moment offsets the fluid resistance torque, and the nanorobot swims steadily with a constant forward velocity where the rotational frequency of the nanorobot synchronizes with the magnetic rotational frequency f; in other words, ω=2πf. In this case, v shows an approximately linear relation against ω when ω<ωₛₜₑₚ₋ₒᵤₜ. For ω=ωₛₜₑₚ₋ₒᵤₜ, v and w reach the maximum. Further increase in f results in the increase in the resistance torque on the nanorobot, while the magnetic moment stops increasing, which leads to a torque imbalance on the nanorobot. Therefore, both the forward and the angular velocities of the nanorobot will decrease to rebalance the resistance torque and the external magnetic moment. The simulation results in FIG. 3I show the capability of capturing and trapping the nanorobots with an external magnetic field (field strength is 64.9 mT, frequency is 3 Hz, swarming motion speed is 0.5 mm/s, and flow speed is 60 mm/s) at both step 1 (downstream process) and step 2 (upstream process).

The study of the parameters affecting the recapturing efficiency indicates that the recapturing efficiency decreases with the decrease of the magnetic field strength, the increase of the flow speed in the vessel, and the increase of the locomotion distance of the nanorobots inside the vessel. A high recapturing efficiency of the nanorobots can be realized with a wide range of nanorobot concentrations and doses. To ensure optimal recapturing of the nanorobots, the injection speed of the nanorobots from the catheter should be fixed to 0.05 ml/s, as larger injection speeds will cause substantial loss of the nanorobots.

### In Vitro Thrombolysis

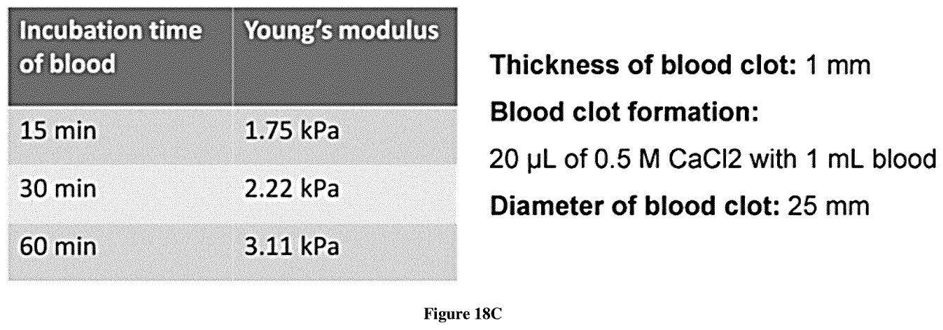

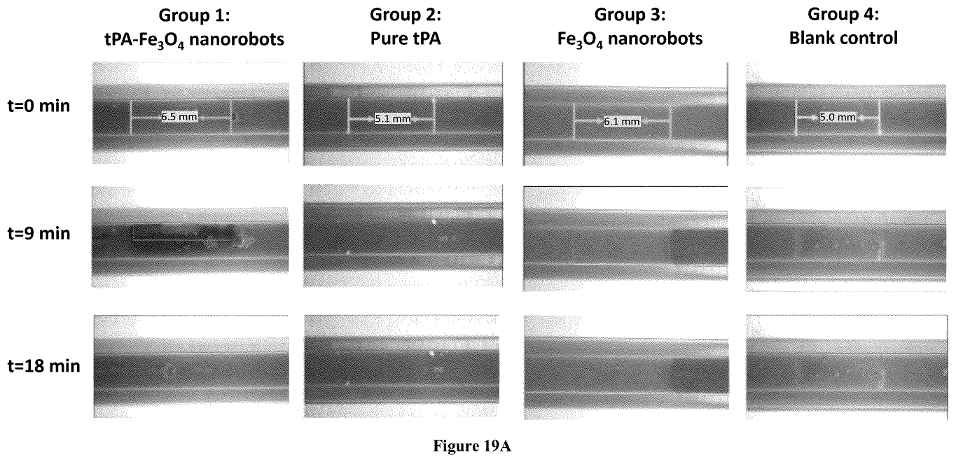

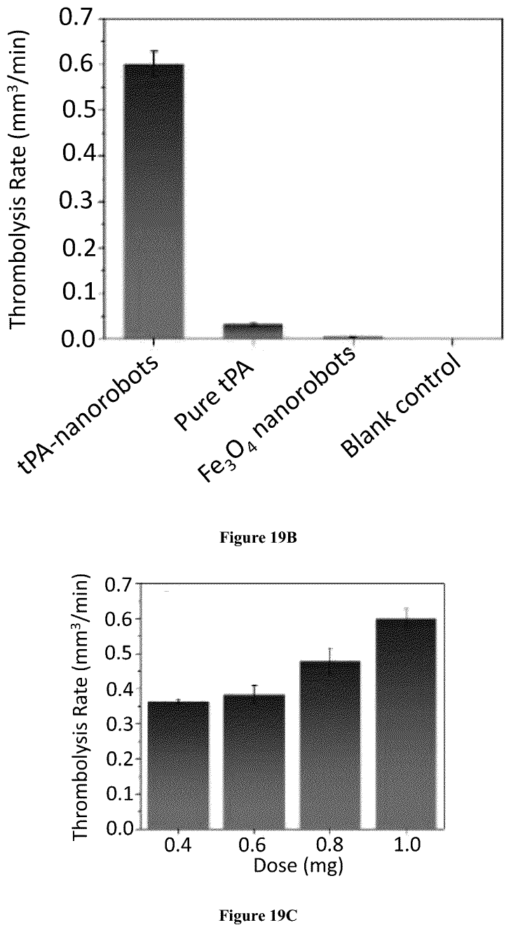

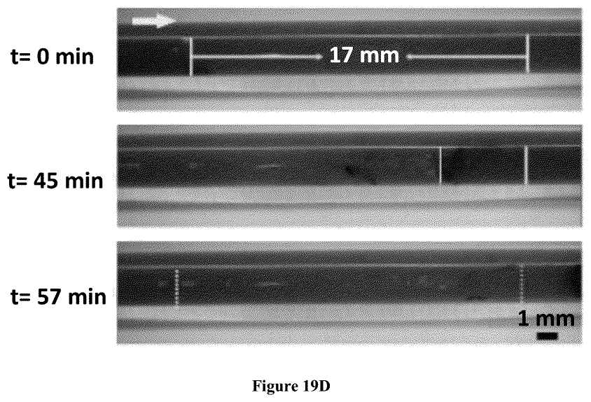

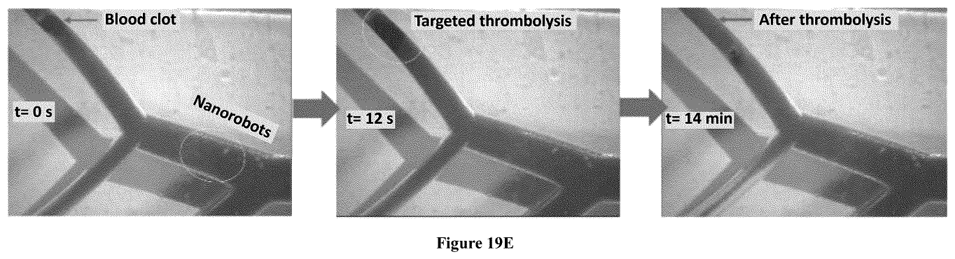

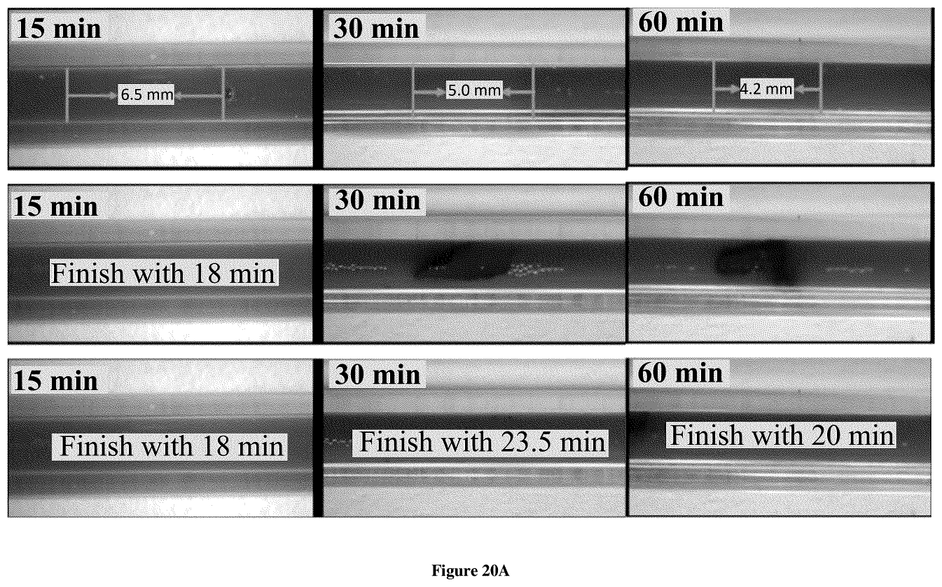

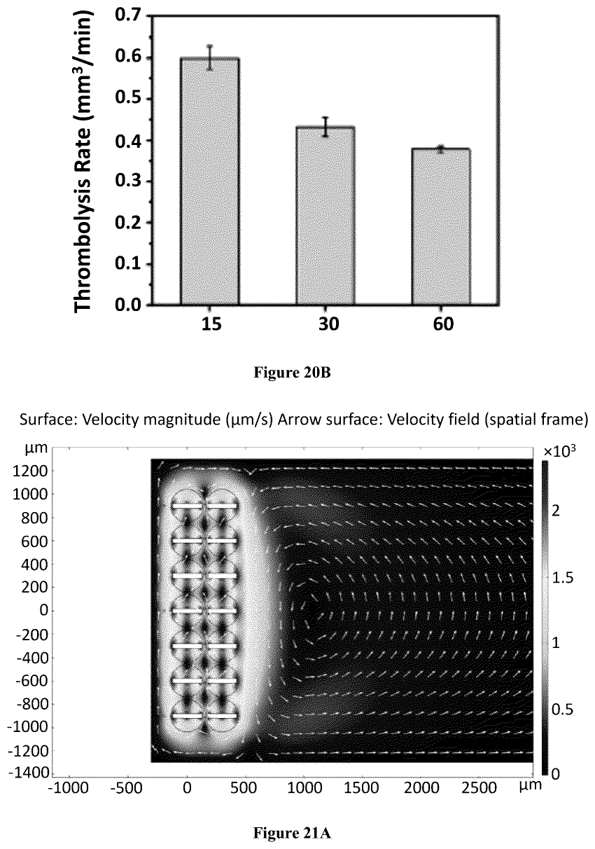

To assess the efficiency of thrombolysis of the prepared tPA-nbots, the thrombolysis process of blood clots in a vessel with a diameter of 1.5 mm, which is comparable to blood vessel of the M3 segment (movie S2: In vitro thrombolysis in a glass tube and a 3D model with whole blood), was investigated. The mechanical property of the blood clot is measured, with Young's modulus in the range of 1.8 to ˜3.1 kPa according to a standard blood clot in situ generation method (FIGS. 18A to 18C). Four control experiments were conducted to investigate the thrombolysis performance of the tPA-nbots in vessels filled with blood (FIGS. 19A to 19E). For group 1, 1 mg of tPA-nbots was administered. The dose of tPA on the nanorobots was calculated to 15 μg. The original length of the artificial blood clot was 6.5 mm, and more than half of the clot was degraded by the action of tPA from tPA-nbots after 9 min. It took 18 min to fully remove the blood clot using tPA-nbots, and the thrombolysis rate was around 0.638 mm³/min. In group 2, the thrombolysis of solely 625 μg of tPA was investigated, and it was found to be much lower than that of tPA-nbots. Only a small part of the blood clot was lysed by the tPA after 18 min, and the thrombolysis rate was calculated to ˜0.0318 mm³/min. Compared with pure tPA, the dose of tPA on tPA-nbots is reduced by about 42-fold, yet the thrombolysis rate is improved about 20 times (FIG. 19B). In addition, as the modulus of a blood clot can be controlled within 1.8 to ˜3.1 kPa, the thrombolysis efficiency was further compared by using the prepared tPA-nbots with various moduli. The results indicate that the proposed tPA-nbots is capable of removing blood clots with different moduli, although the thrombolysis rate shows a slight decrease as the blood clot becomes stiff (FIGS. 20A and 20B). As the dose of the tPA-nbots increased, the thrombolysis speed increased proportionally. Besides, it was demonstrated that long blood clots with large aspect ratios (i.e., experimentally done up to about 11) can be removed with the tPA-nbots, as depicted in FIG. 19D, and a similar high thrombolysis rate of 0.53 mm³/min was observed. Notably, such nanorobots are capable of performing targeted thrombolysis in complex three-dimensional (3D) vessels, and it takes ˜15 min to realize recanalization of the 3D blocked vessel (blood clot length was 3 mm), as shown in FIG. 19E. The capability to navigate in 3D vessels is important as human blood vessels have a 3D vascular distribution.

Mechanism of the Thrombus Removal by Using tPA-Nbots

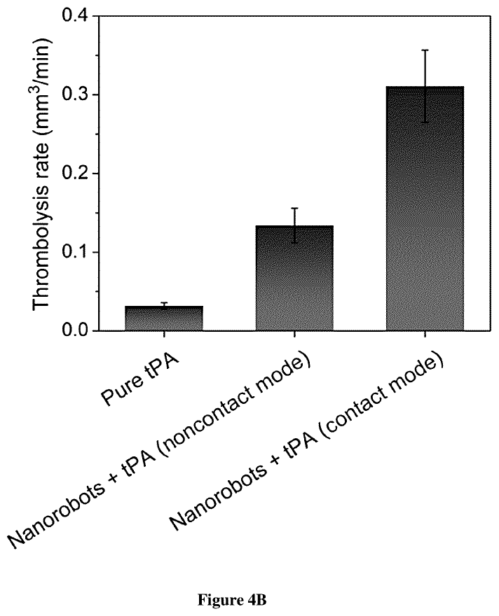

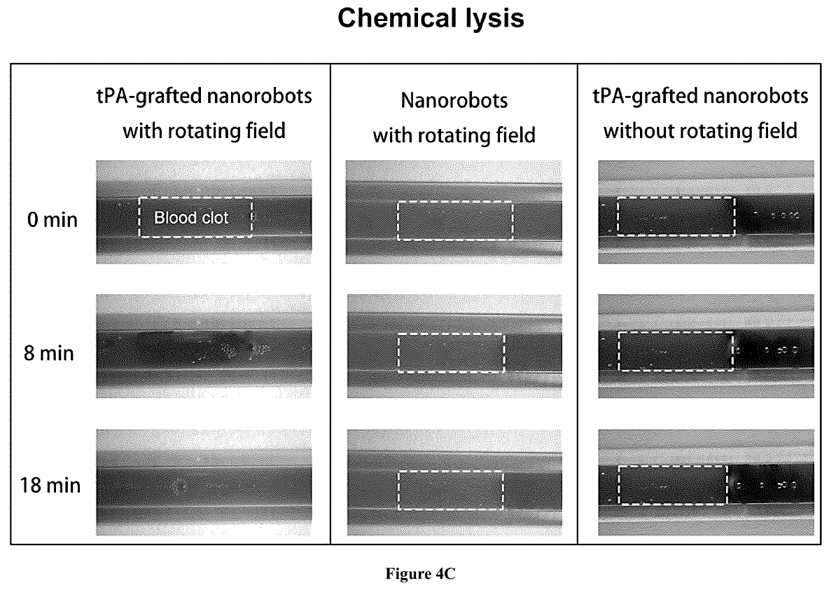

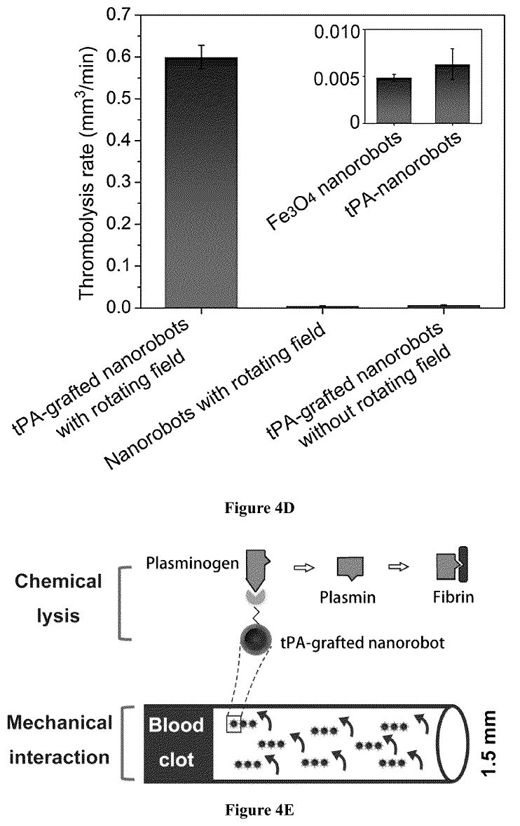

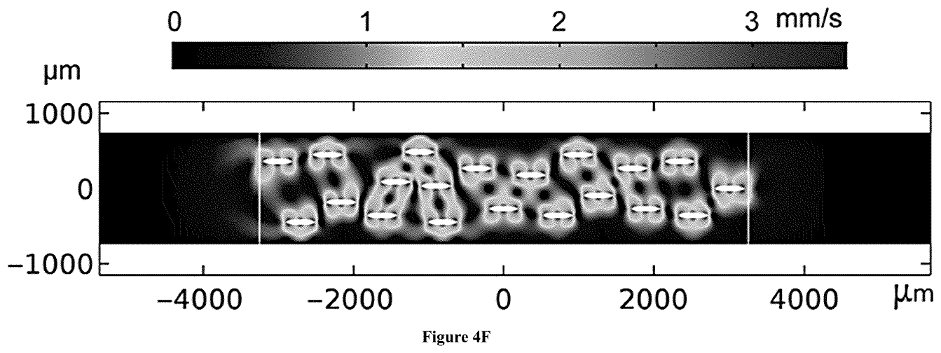

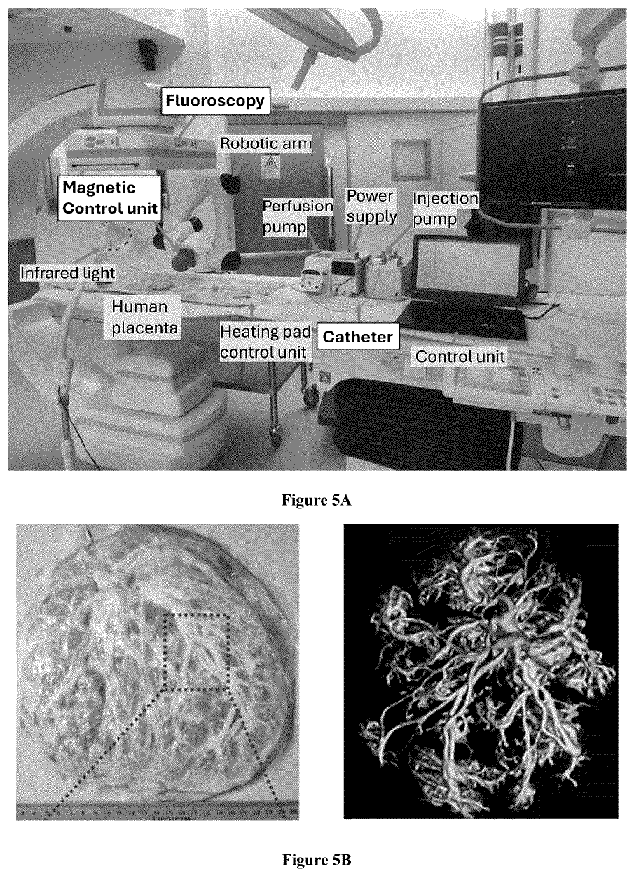

The combined effect of mechanical interaction and chemical lysis shows a substantially enhanced thrombus removal efficiency, with a recanalization time 12 times larger than that of only chemical lysis (pure tPA group in FIG. 4B) and 120 times larger than that of only mechanical interaction (Fe₃O₄ nanorobots group in FIG. 4D). The rapid removal of the blood clot is derived from the combined effect of mechanical interaction and chemical etching. As shown in FIGS. 4A and 4B, with high tPA concentration (0.65 mg of tPA), Fe₃O₄ nanorobots with mechanical interaction and dynamic convection show a 10-fold higher thrombolysis rate compared to pure tPA. In comparison, nanorobots with only dynamic convection show about 4.2-fold increases in thrombolysis rate. Therefore, mechanical interaction and dynamic convection contribute greatly to the thrombolysis process. Chemical etching is another important factor that improves thrombolysis. As shown in FIGS. 4C and 4D, tPA-nbots without the RMF show a marginal thrombolysis rate (0.0063 mm³/min), illustrating the chemical etching of tPA-nbots without mechanical interaction is inefficient. The thrombolysis rate of Fe₃O₄ nanoparticles that merely show the mechanical interaction with the RMF is even lower with 0.0049 mm³/min than that of tPA-nbots with the RMF. Therefore, tPA-nbots with both mechanical and chemical etching exhibit the highest thrombolysis rate and save abundant recanalization time.

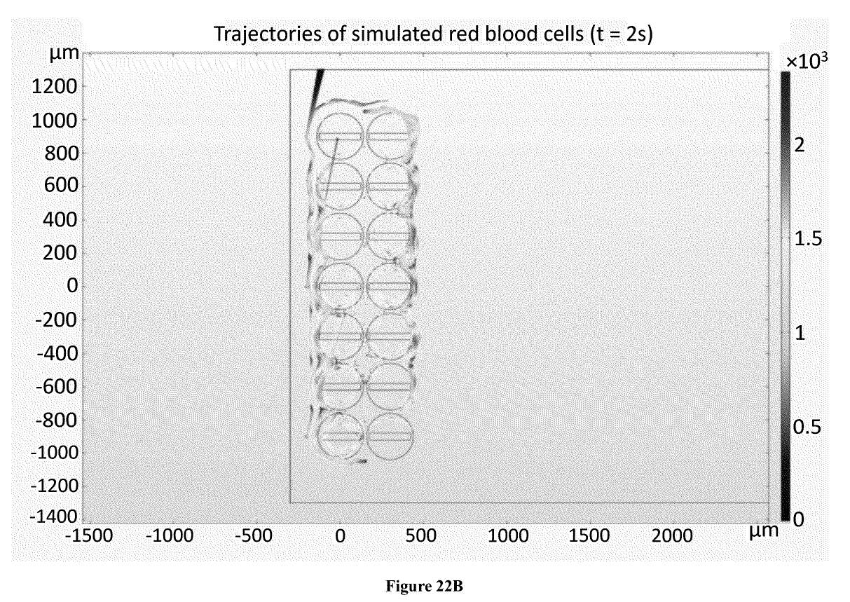

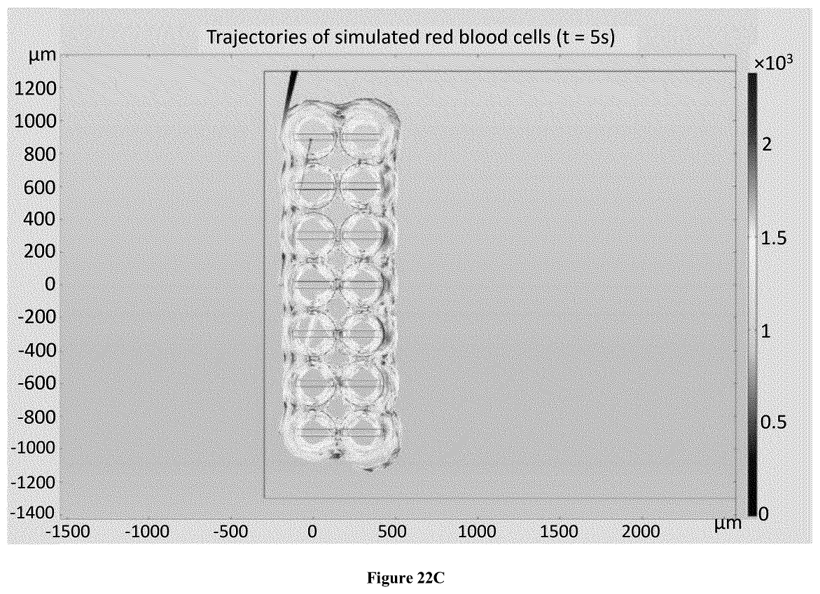

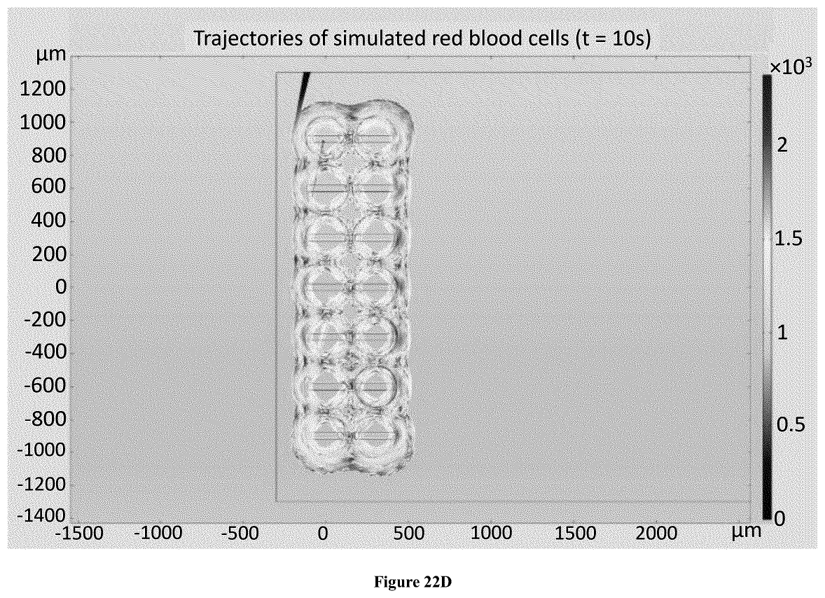

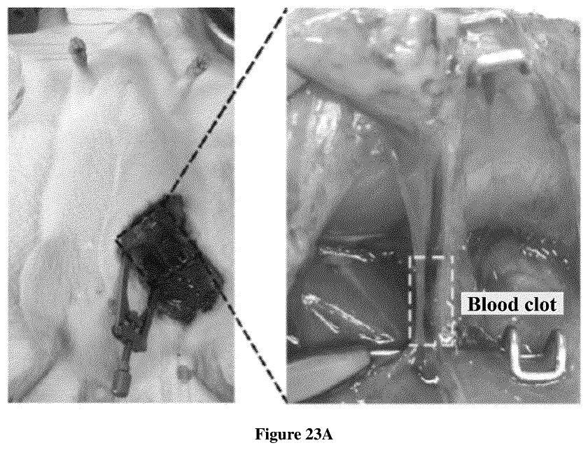

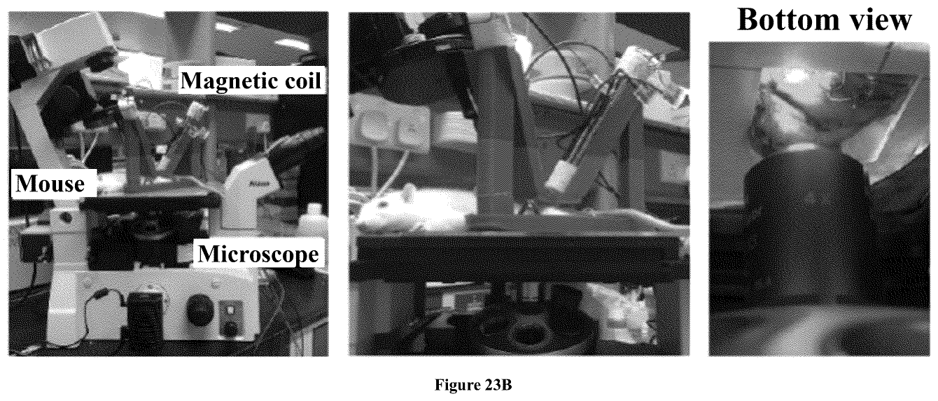

As shown in the scheme in FIG. 4E, the high thrombolysis rate by using the tPA-nbots is achieved by the synergy of mechanical interaction and chemical etching. The mechanical rotation and scraping not only increase the contact probability of the nanorobots and the blood clot but also enhance the dynamic convection around the nanorobots (FIG. 4F). The chemical etching is performed by the surface-anchored tPA molecules (FIG. 4E). The surface-anchored tPA catalyzes the transition of PLG to PLM, which binds to fibrin and breaks up the blood clot in blood vessels to recover the blood flow. FIGS. 21A to 21C and 22A to 22D give the simulation results of the swarm pattern-induced flow profile near a clot-fluid interface and the motion of red blood cells around the swarm pattern. The simulation results suggested that the nanorobots generate enhanced mechanical pressure force and fluid convection around them, facilitating the thrombolysis.

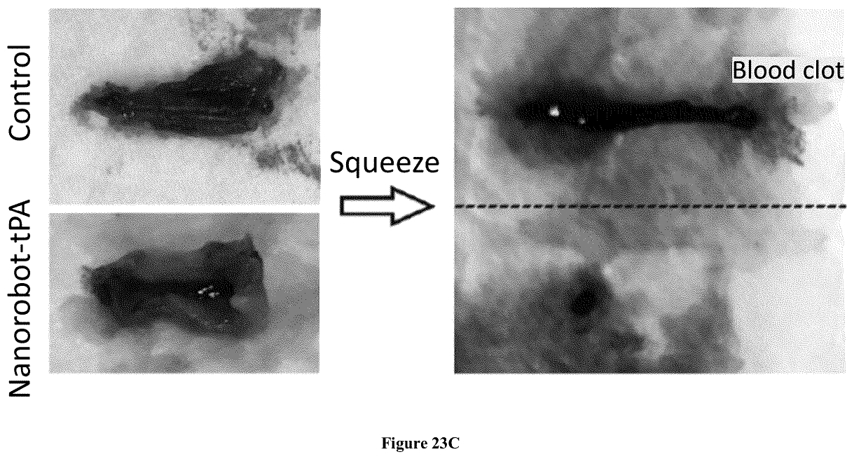

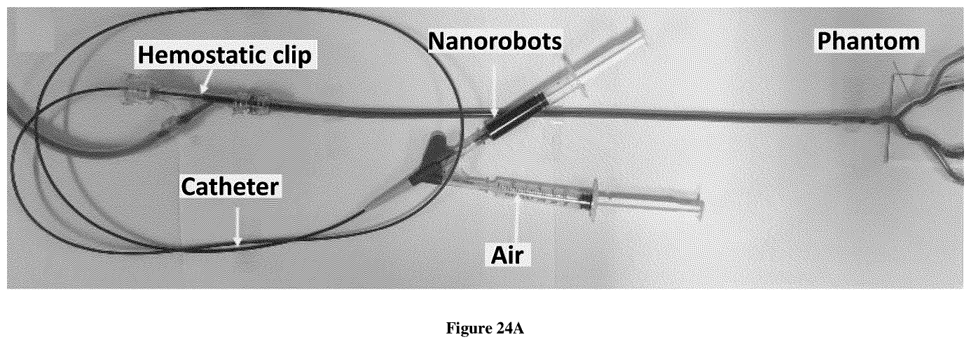

To verify the enhanced thrombolysis by the tPA-nbots, the in vivo experiment was performed on rats with in situ-induced blood clots in the femoral vein (FIG. 23A). One hundred microliters of tPA-nbots (6 mg/ml) were injected into the vessel about 1 cm away from the blood clot. Subsequently, the rat was placed under the magnetic field setup with a magnetic field of about 10 mT in the vicinity of the blood clot (FIG. 23B). The pitch angle of the magnetic field was set to 90° to make sure that the nanorobots rotated toward the clot. As a control, another rat with blood clot was not injected with the tPA-nbots. After 1 hour of treatment with the magnetic field, the vessels of both rats were cut, as shown in FIG. 23C. Both vessels were squeezed with a tweezer to observe the blood clot inside the vessel. The blood clot in the rat with tPA-nbot treatment disappeared, whereas the ˜ 1-cm blood clot in the rat without tPA-nbot treatment remained intact, as shown in FIG. 23C. These results verify the excellent capability for in vivo thrombus lysis with the combination of mechanical interaction and chemical lysis.

### In Vitro Thrombolysis and Retrieval of Nanorobots

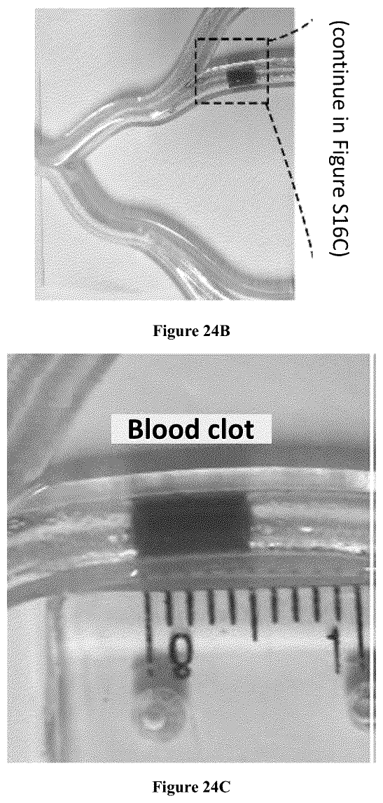

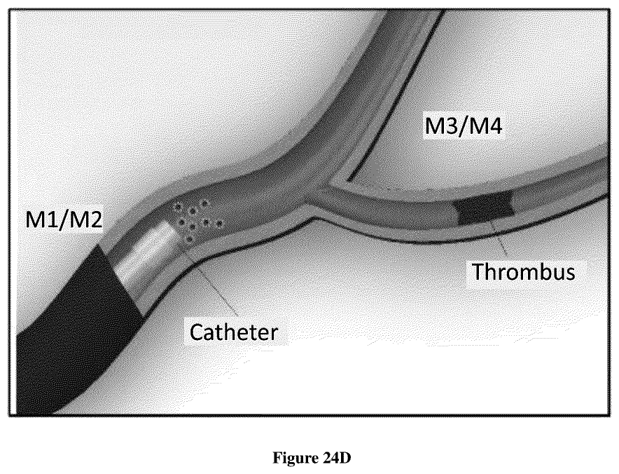

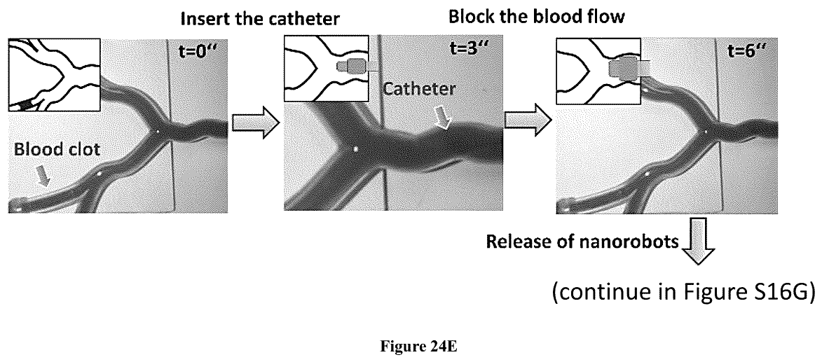

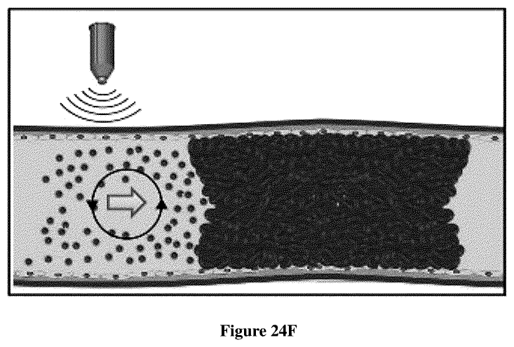

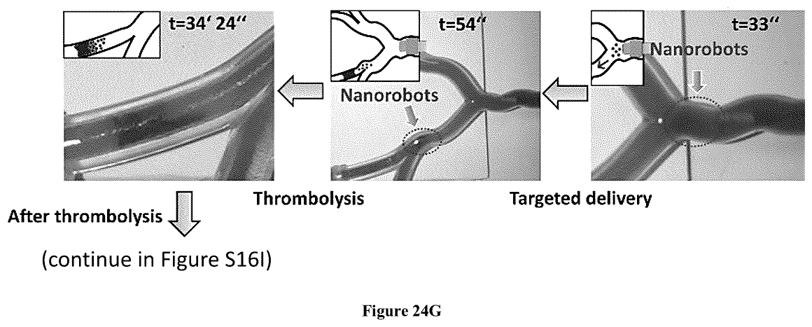

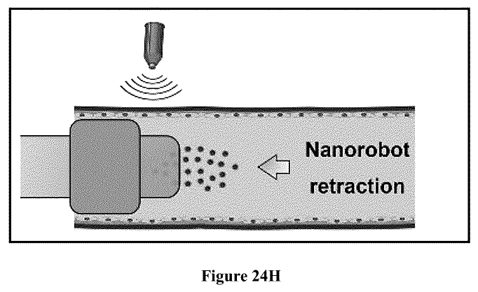

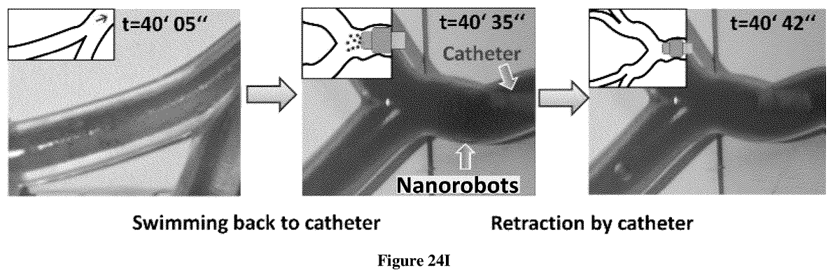

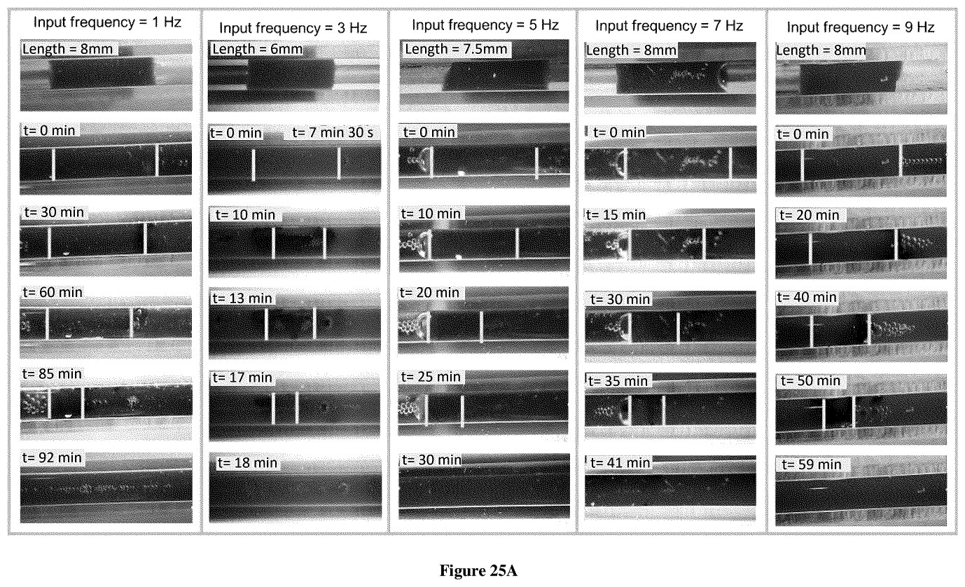

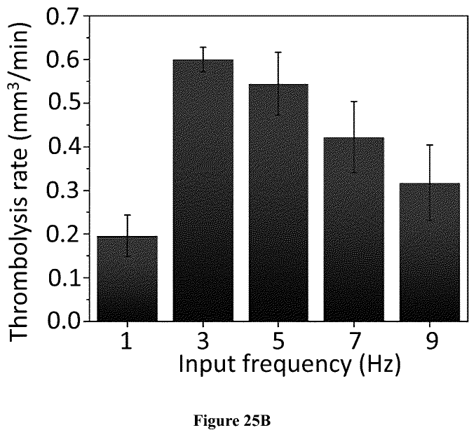

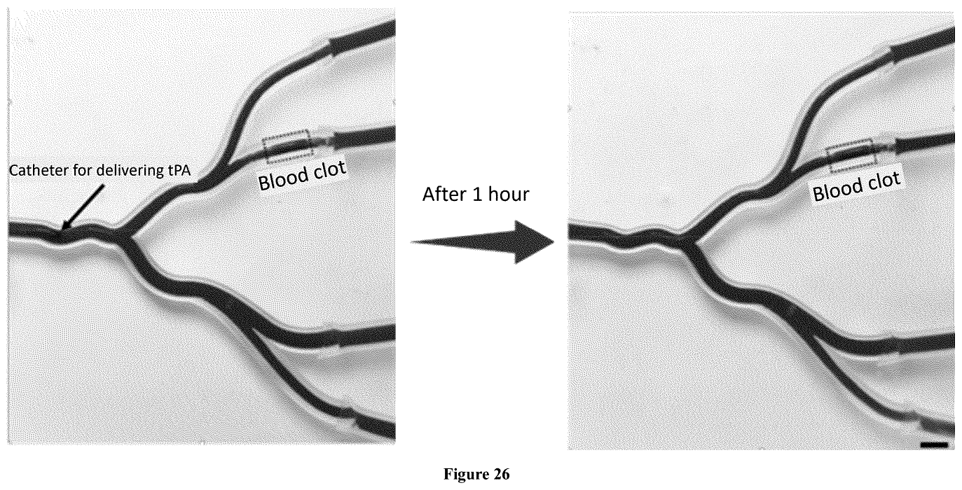

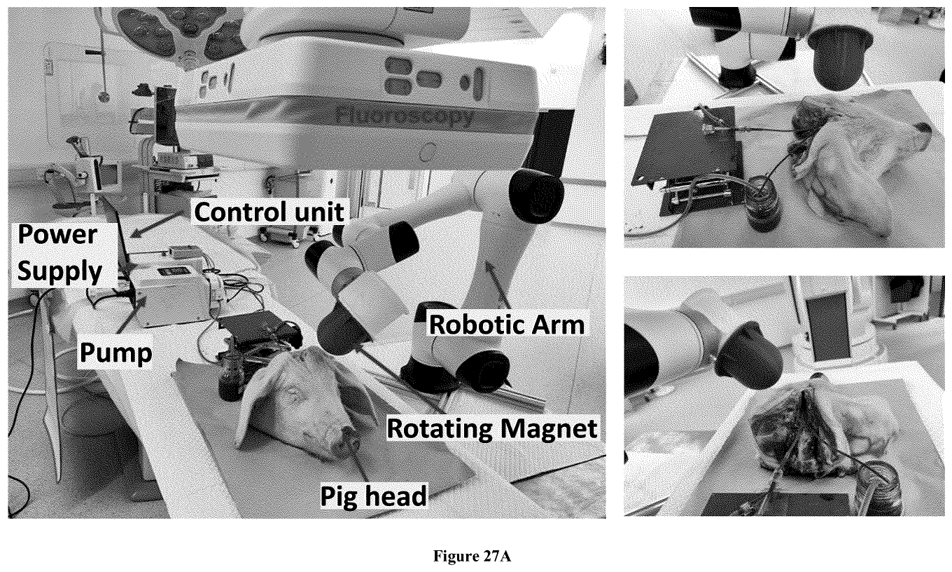

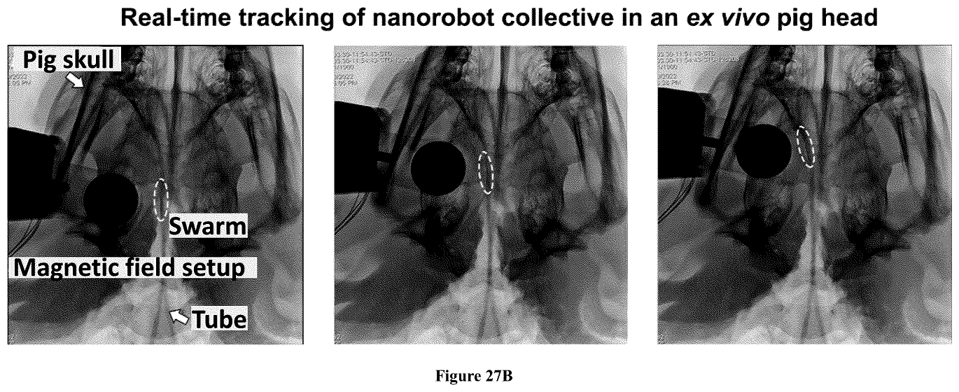

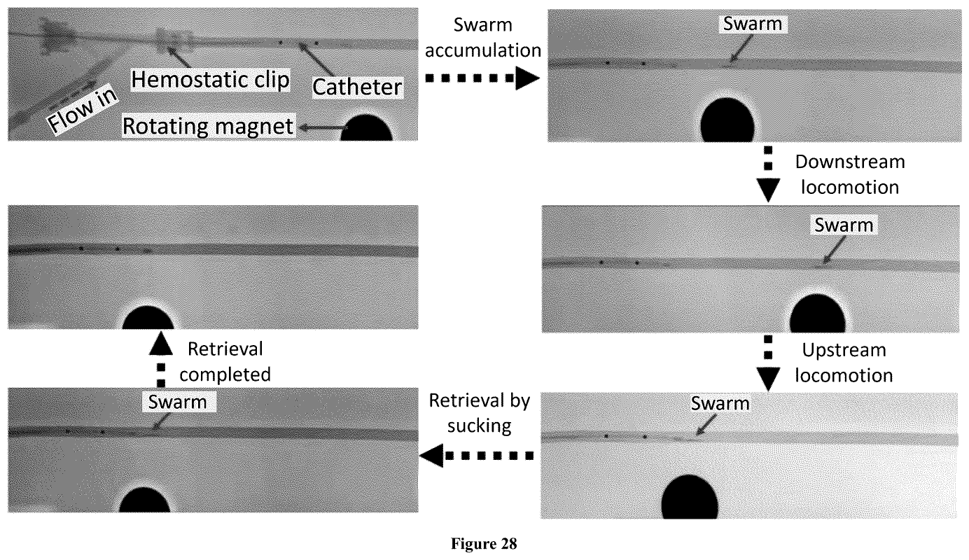

To demonstrate the targeted thrombolysis and fast retrieval of the tPA-nbots, an in vitro vascular model with blood flow was established, as shown in FIG. 24A, to verify the mechanical and chemical synergetic thrombolysis in combination with the ability to recapture the nanobots (movie S3: Targeted thrombolysis using tPA-nanorobots with retrieval ability in a phantom). A blood clot was induced in a branched channel with a diameter of 2 mm and a length of 5 mm (FIGS. 24B and 24C). The blood flow was established with a peristaltic pump. The catheter-assisted magnetic navigation of tPA-nbots for high-precision delivery and thrombolysis was performed, followed by rapid recapturing of the colloidal microswarm. To ascertain the optimal conditions for thrombolysis, a magnetic field strength of 65 mT and various input frequencies were used. The findings reveal that the thrombolytic speed of tPA-nbots attains its peak at a magnetic field frequency of 3 Hz (FIGS. 25A and 25B). Upon interaction with a thrombus, swarming tPA-nbots experience asymmetrical forces, particularly encountering substantial resistance on one side, possibly elucidating the observed lower optimal frequency compared to the previously established maximum swarming frequencies on flat and curved surfaces (FIGS. 15A to 15M). Catheter-assisted magnetic navigation and retrieval of tPA-nbots for targeted thrombolysis in a 3D in vitro channel is conducted. As schematically shown in FIG. 24D, the catheter was inserted into the main vessel of the dummy vessel, followed by blocking the vessel via a balloon attached to the terminal of the catheter. Subsequently, the tPA-nbots were released into the main vessel with a dose of ˜ 1.3 mg. By the magnetic navigation, the tPA-nbots were actuated to the blood clot and performed the on-demand etching and lysing of the blood clot from the inner wall of the vessel by the synergetic effect of mechanical and chemical etching, and the thrombolysis rate is 0.403 mm³/min (FIGS. 24F and 24G). After ˜39 min, the blood clot was completely removed, and the nanorobots were actuated back toward the catheter in swarming motion, as schematically shown in FIG. 24H. Once the microswarm reached the terminal of the catheter, it was sucked into the catheter (recaptured/trapped) with a retrieval rate of 91.91%, as shown in FIG. 24I. Last, the catheter was removed from the dummy vessel, and the blood flow inside the blocked vessel was recanalized. As a control, the thrombolysis effect was also tested by directly releasing tPA at the end of the catheter. The result in FIG. 26 indicated that no obvious thrombolysis effect was observed when tPA was directly delivered by catheter at the site close to the blood clot branch, although the dose of pure tPA is 100-fold compared with the tPA dose of 1 mg nanorobots. The passive diffusion of tPA to the blood clot results in the limited thrombolysis effect because the branch was fully blocked. By compassion, the tPA-nanorobots could actively locomote to the targeted blood clot for performing efficient thrombolysis.

### Ex Vivo Thrombolysis in Human Placenta and Retrieval of Nanorobots

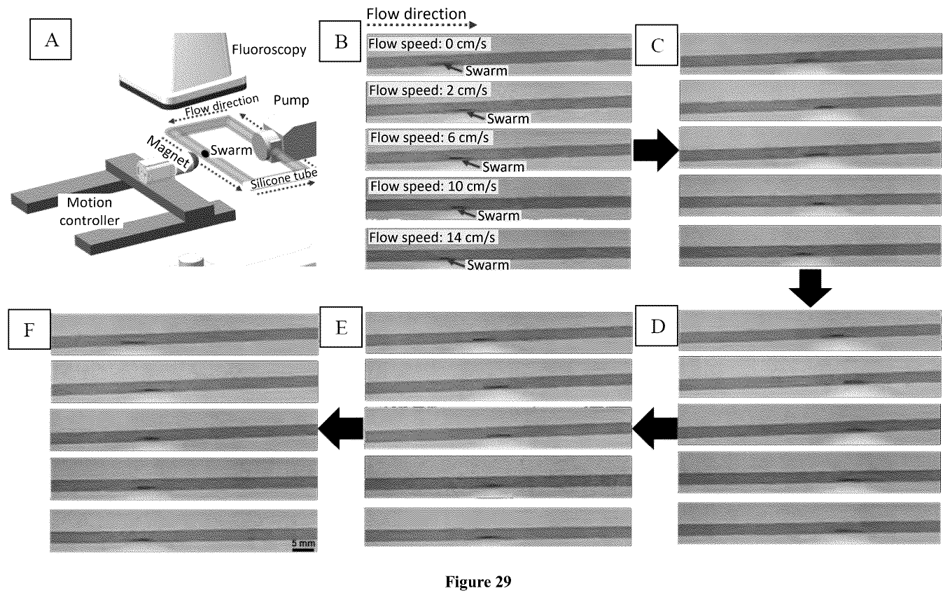

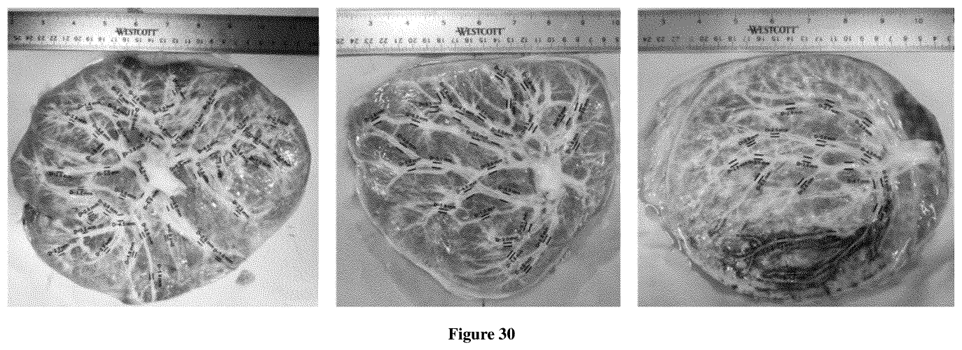

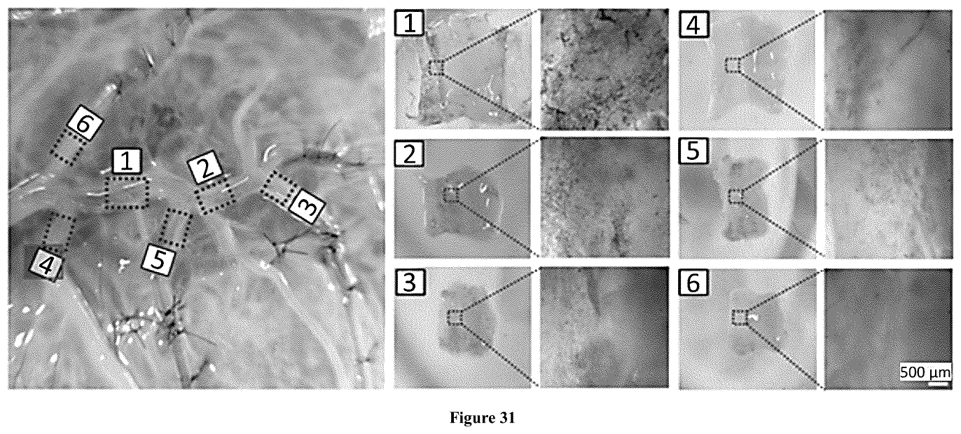

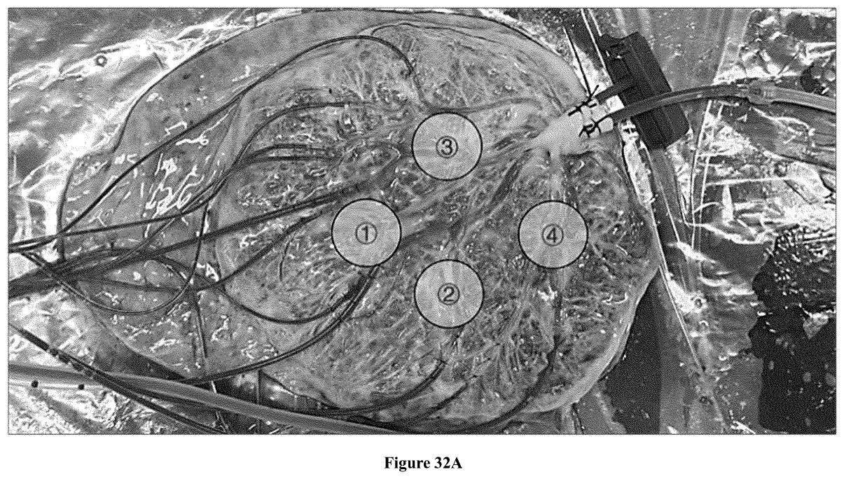

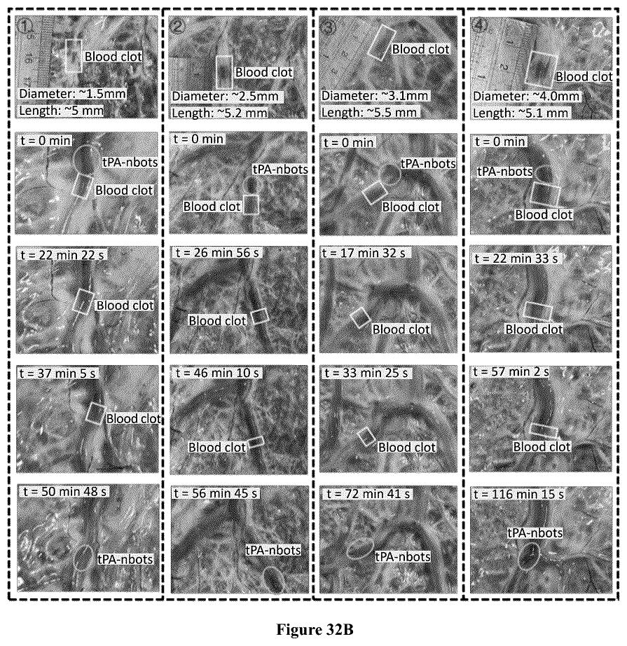

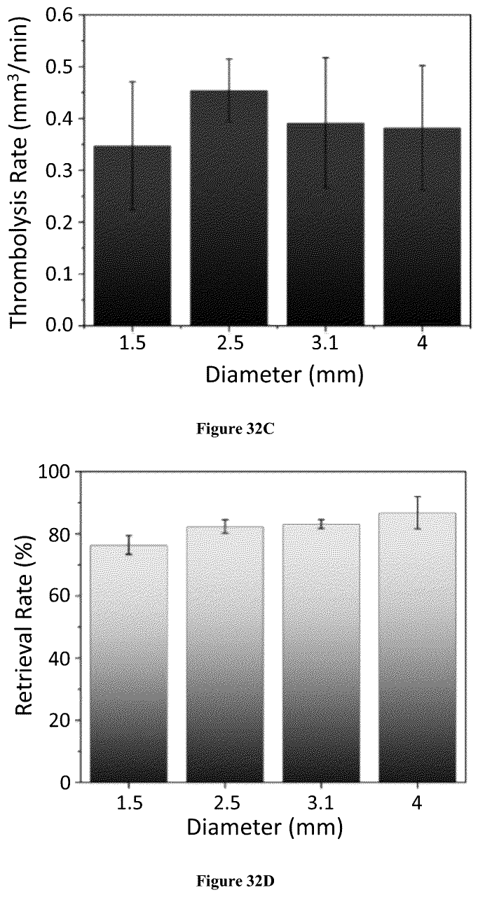

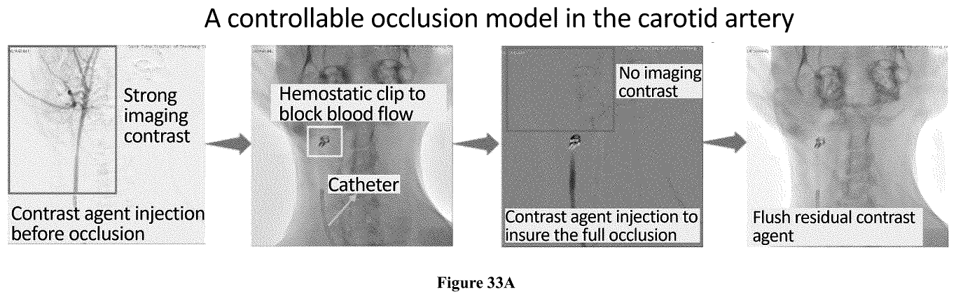

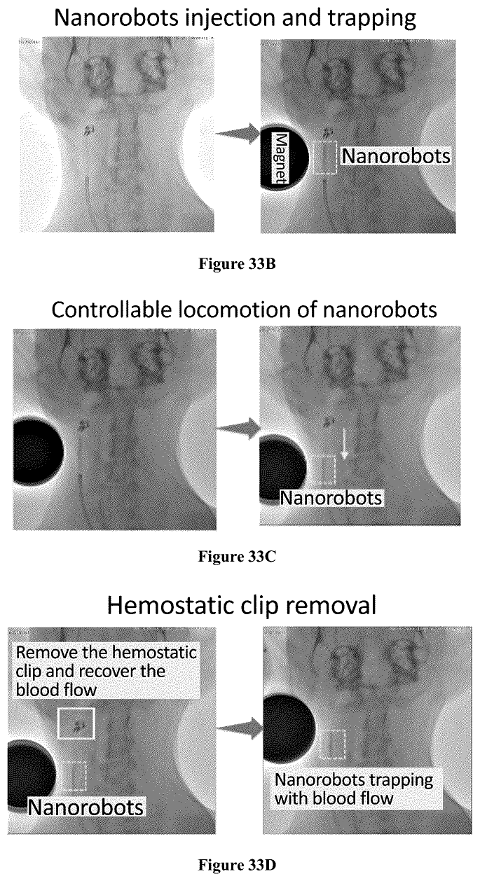

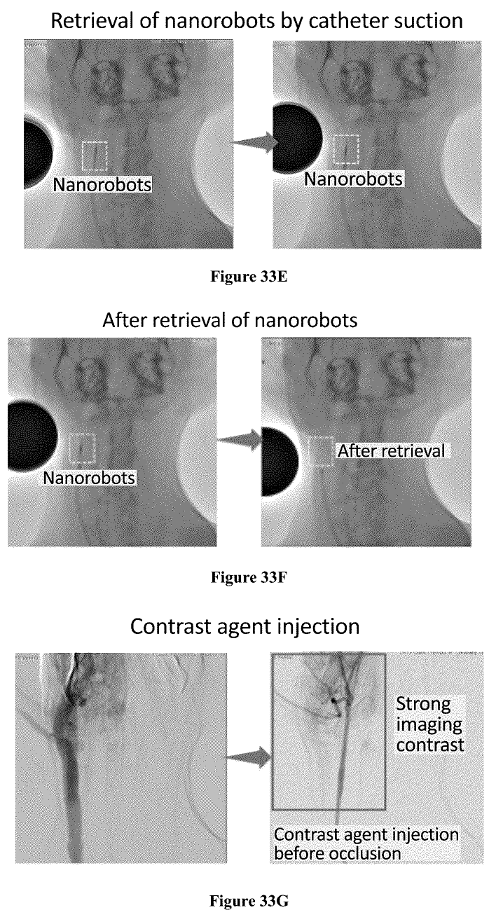

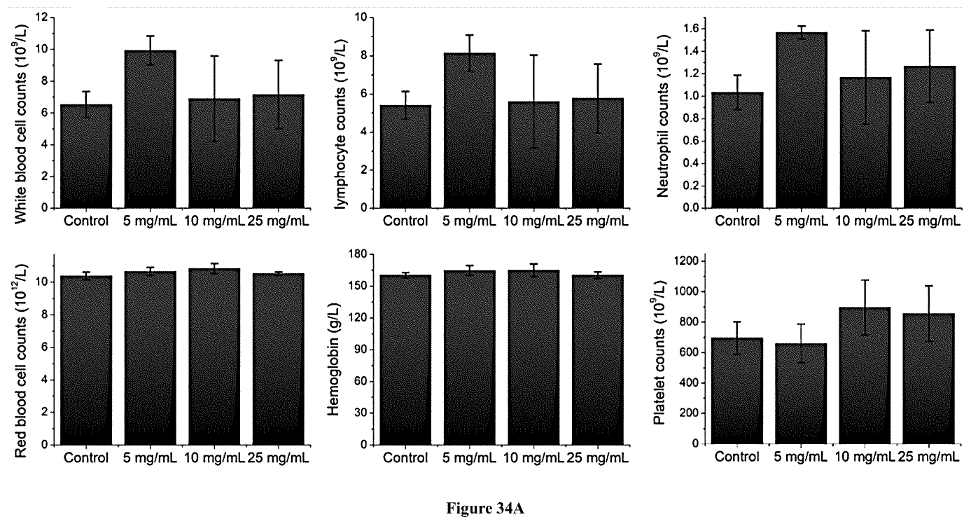

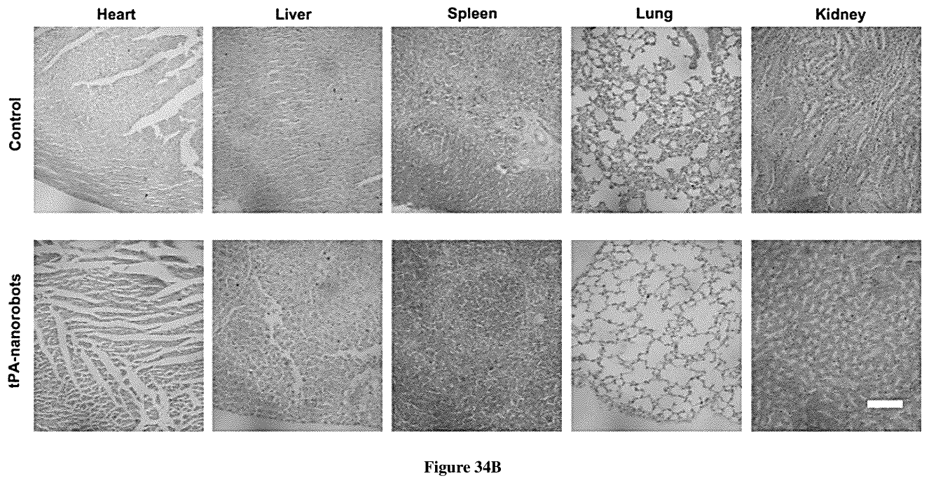

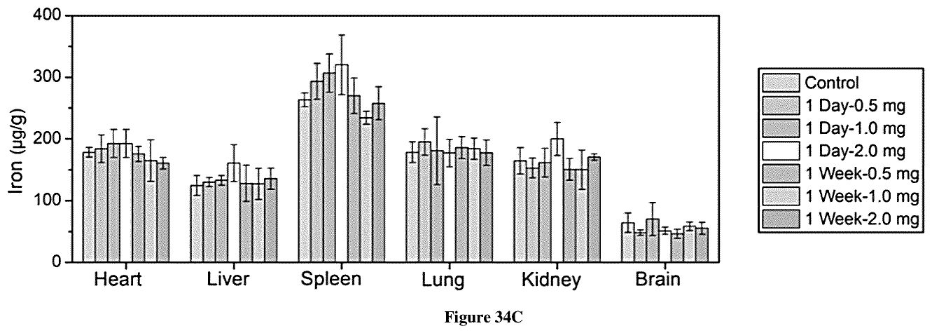

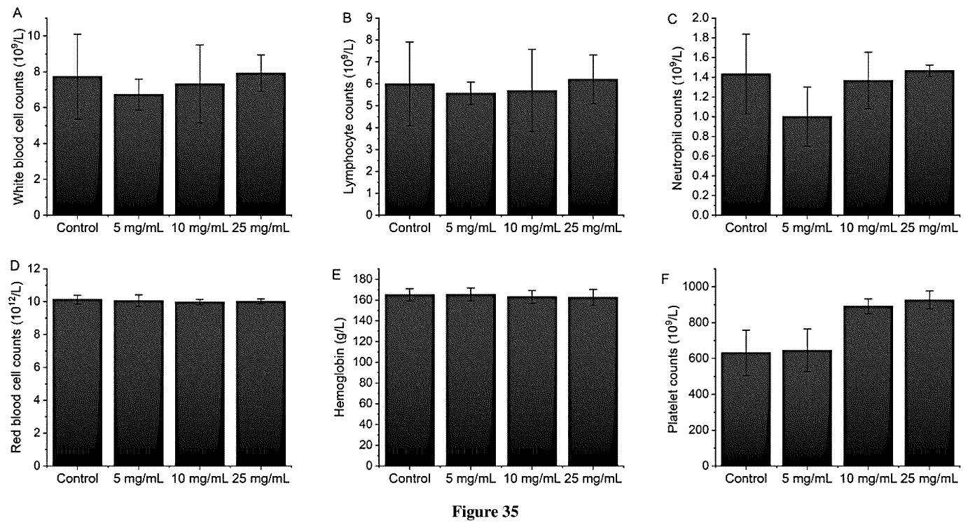

Human placenta shows a comparable vessel distribution to the human brain vessel distribution. Moreover, the placenta as an ideal model has been broadly used for human brain vascular microneurosurgery training such as tumor and aneurysm surgery (45). Here, a human placenta model with a blood clot occurring at a 1.5-mm (diameter) vessel to simulate an ischemic stroke at an M3/M4 segment was established. Similar to the experiment before, the blood flow is established with a peristaltic pump. Catheter-assisted magnetic navigation in combination with x-ray fluoroscopy tracking system (CMAFIS)-comprising a catheter, mechanical arm equipped with a magnetic locomotion unit, and x-ray fluoroscopy setup—enables the rapid thrombolysis and recapturing of tPA-nbots from the vessels (FIG. 5A). The colloidal microswarm can be tracked as an entity due to the submillimeter-scale spatial resolution of the fluoroscopy with exceptional penetration capability of various vessels, tissues, and bones. It was verified that the colloidal microswarm can be clearly tracked in a real-time manner with fluoroscopy during the magnetic navigation in a pig head, with the pig skull covered over the microswarm (FIGS. 27A and 27B and movie S4: Fluoroscopy imaging of tPA-nanorobots swarm in a pig head). The developed CMAFIS was used to realize the transvascular delivery of the tPA-nbots toward the blood clot for precise treatment by means of magnetic control, while the fluoroscopy-tracking yielded real-time feedback (FIG. 26). Moreover, there is no obvious decline in the imaging contrast of microswarm with different blood flows ranging from to 14 cm/s (FIG. 29). A human placenta with an artificial blood clot of 6 mm at a 1.5-mm branched vessel was established (FIG. 5B and movie S5: Fluoroscopy imaging-guided thrombolysis in human placenta by using tPA-nanorobots microswarm with retrievable capability). FIG. 5C shows the placenta with a 6-mm blood clot before the retrievable thrombolysis, and FIG. 5D shows the placenta after the retrievable thrombolysis. The placenta vessel was cycled with a blood flow of 20 mm/s. The whole treatment process can be divided into three stages. In stage 1, the catheter was inserted into the human placenta model along the direction of blood flow to the vascular branch that is in vicinity to the vessel with the blood clot. This process was observed by real-time tracking with the help of the fluoroscopy imaging setup, as illustrated in FIG. 5E. As the iodine contrast is injected into the blood in advance, one can also find out the blocked blood vessel with case because the blocked vessel without iodine-containing blood shows notably weaker contrast compared with the other vessels. The tPA-nbots (5 mg) were released from the catheter, as shown in FIG. 5F. In stage 2, the tPA-nbots were navigated toward the blood clot by magnetic control (field strength is 65 mT, and frequency is 3 Hz) with a locomotion speed of 0.5 mm/s to induce controlled thrombolysis, while this process was monitored by real-time fluoroscopy, as shown in FIGS. 5G and 5H. The magnetic actuation setup was diagonally vertical to the blood vessel with the angel of about 45° to avoid the occlusion effect of the fluoroscopic imaging. The blood clot was scraped by the mechanical stirring and etched by biochemical lysis, and the thrombolysis rate in the placenta was 0.46 mm³/min. Subsequently, in stage 3, the nanorobots were magnetically propelled back toward the catheter (FIGS. 51 and 5J). As the microswarm reached the terminal of the catheter, it was sucked into the catheter, and the catheter was pulled out of the vessel to complete the vascular recanalization (FIG. 5J). One can clearly observe that the blood flow is recovered after the fluoroscopy-tracked treatment with tPA-nbots.

The retrieval capability of nanorobots in the placenta, with liquid flows (1× diluted blood) and multiple branches quantitively, was further evaluated as shown in FIGS. 5K to 5N. Three representative placentas used in experiments are shown in FIG. 30. The placentas show complex and torturous blood vessel distribution, and the diameter of the blood vessel ranges from 1.0 to 5.5 mm. The retrieval percentage of the nanorobots by the catheter-assisted magnetic navigation system increased with the increase of the injection dose, and all maintained more than 70%. For a commonly used dose of 3 mg, the percentage of recaptured tPA-nbots reaches up to ˜80% of the originally injected ones (FIG. 5K; injection speed, 0.05 ml/s; concentration, 2 mg/ml; dose, 1 to 5 mg; field strength, 64.9 mT; flow speed, 1.5 to 5.2 cm/s; distance between magnet centra and catheter tip, ˜1.5 cm; locomotion distance, ˜3 cm). For the change of the magnetic field strength, the retrieval percentage is similar with a magnetic field strength of 121.5 and 64.9 mT, around 80%, while no nanoparticles can be recycled from the placenta with a magnetic field strength of 22.4 mT (FIG. 5L; injection speed, 0.05 ml/s; concentration, 2 mg/ml; dose, 2 mg; flow speed, 1.5 to 5.2 cm/s; distance between magnet centra and catheter tip, ˜1.5 cm; locomotion distance, ˜3 cm). The retrieval percentage slightly decreases with the increase of the locomotion distance in the blood vessel. As shown in FIG. 5M, it still remains at about 71.1% even after 14-cm locomotion distance (injection speed, 0.05 ml/s; concentration, 2 mg/ml; dose, 2 mg; field strength, 64.9 mT; flow speed, 1.9 to 5.2 cm/s; distance between magnet centra and catheter tip, ˜1.5 cm; locomotion distance, 6 to 14 cm). The retrieval percentage of the tPA-nbots decreased with the decrease of the flow speed in the placenta (FIG. 5N; injection speed, 0.05 ml/s; concentration, 2 mg/ml; dose, 2 mg; field strength, 64.9 mT; distance between magnet centra and catheter tip, ˜1.5 cm; locomotion distance, ˜3 cm). The retrieval percentage can be as high as around 90% with a lower flow speed of 0.5 to 3.4 cm/s and maintained at 50% with a high flow speed of 3.5 to 12.1 cm/s. While the flow speed exceeds 12.9 cm/s, the nanorobots can hardly be trapped. Here, the flow speed is a range because there are lots of vessels in the placenta and the vessel diameter affects the flow speed. In addition, the nanorobots show no serious adhesion to the blood vessel wall after treatment (FIG. 3I). The thrombolysis ability and retrieval percentage of tPA-nbots in the blood vessel with various diameters were also investigated (FIGS. 32A to 32D). Comparable thrombolysis rates can be observed in blood vessels with diameters from 1.5 to 4.0 mm, indicating that the thrombolysis ability of tPA-nbots shows no obvious decline in the diverse blood vessel. The retrieval rate improved from 76.39 to 86.75% with the blood vessel diameter increasing from 1.5 to 4.0 mm, meaning that the larger blood vessel diameter benefits the retrieval of tPA-nbots.

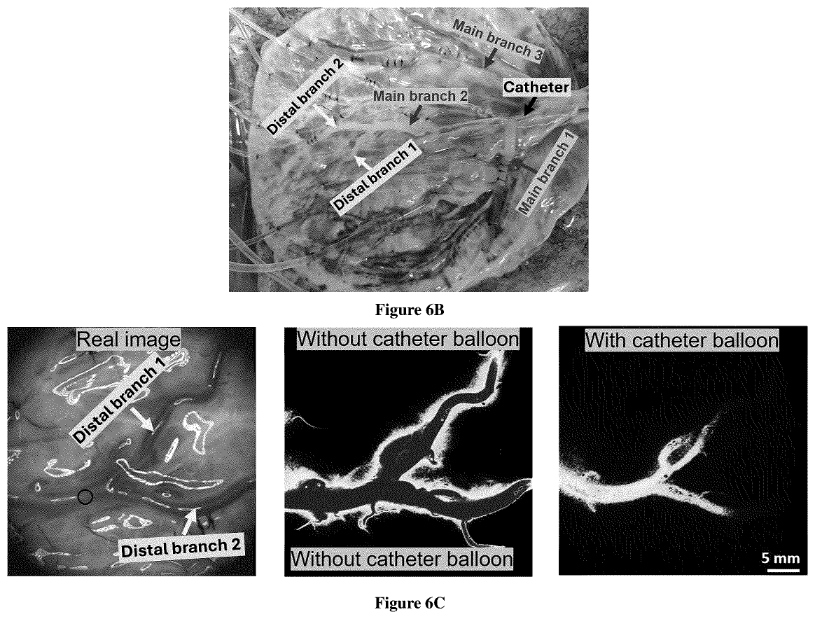

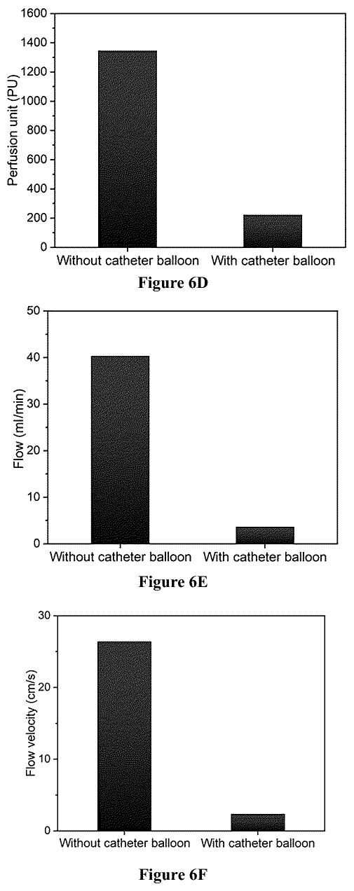

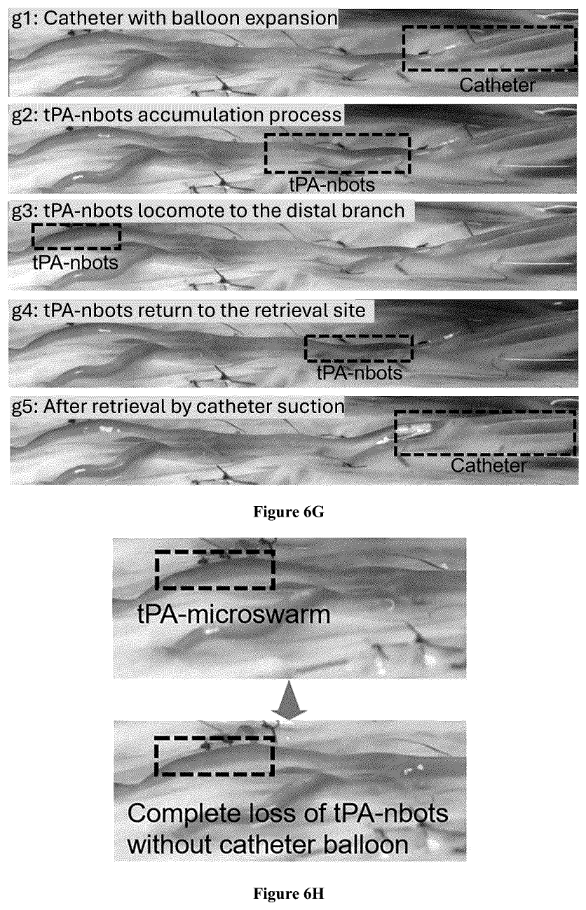

The blood flow velocity in the cerebral system can be as high as more than 21 cm/s, which leads to failure retrieval of the tPA-nbots. Adopting a proper strategy to enable the retrieval of the tPA-nbots with high blood flow conditions is critical for targeted treatment in the cerebral system. Therefore, the catheter balloon was further used to reduce the blood flow and further achieve the targeted locomotion and retrieval of the tPA-nbots in the placenta model. FIG. 6A shows the placenta used in the present experiment with blood vessel diameters ranging from 1.5 to 5.5 mm. The selected blood vessel distribution of the placenta is exhibited in FIG. 6B, and all the main branches and distal branches were connected to each other with attached silicone tubes at the end to ensure the free flow of the blood. Main branch 2 was used for catheter balloon intervention due to its relatively long and torturous blood vessel distribution, while main branch 1 and main branch 3 were kept unimpeded during the whole process. To evaluate the effectiveness of the catheter balloon in reducing blood flow, a laser speckle blood flow imaging system (LSBFIS) was used to measure the blood flow change in the placenta both before and after the catheter balloon expansion. The pseudo-color patterns of the blood vessels depict changes in imaging signal intensity with and without the catheter balloon (FIG. 6C). It is evident from the patterns that the imaging signal intensity weakened after the catheter balloon intervention, indicating a substantial reduction in blood flow. The quantitative result in FIG. 6D shows that the perfusion unit decreased from 1344.59 to 220.88 PU after the catheter balloon intervention, and the flow reduced from 40.28 to 3.59 ml/min (FIG. 6E). The mean blood flow velocity in the distal branch 2 was further calculated (FIG. 6F), and the mean blood flow velocity was 2.35 cm/s with the catheter balloon intervention, while the mean blood flow velocity was as high as 26.38 cm/s without the catheter balloon intervention. Thus, the findings confirm that catheter balloon intervention is a viable and effective approach to reduce blood flow and aid in the retrieval of tPA-nbots in the placenta model. Furthermore, the demonstration of retrieval of the tPA-nbots in the placenta with high blood flow was conducted by using catheter balloon intervention (FIG. 6G). Specifically, a catheter with a balloon was expanded in the main branch 2 of the placenta (FIG. 6G, g1), and the tPA-nbots were then released from the catheter, followed by the accumulation process (FIG. 6G, g2). Then, the tPA-nbots were navigated to the narrow distal branch 2, which was hard for the catheter to reach (FIG. 6G, g3). After that, the tPA-nbots returned to the retrieval site controlled by the RMF (FIG. 6G, g4), and the tPA-nbots can be fully retrieved by catheter suction, as observed in FIG. 6G (g5). Compared with catheter balloon intervention, the tPA-nbots were unable to be retrieved because they were completely lost in the narrow distal branch due to the high blood flow (FIG. 6H). The retrieval rate of the tPA-nbots with and without the catheter balloon intervention was compared and presented in FIG. 6I, and approximately 84.7% of tPA-nbots were successfully retrieved with the catheter balloon intervention, proving the validity of using the catheter balloon to reduce the blood flow and enable the retrieval process.

### In Vivo Thrombolysis

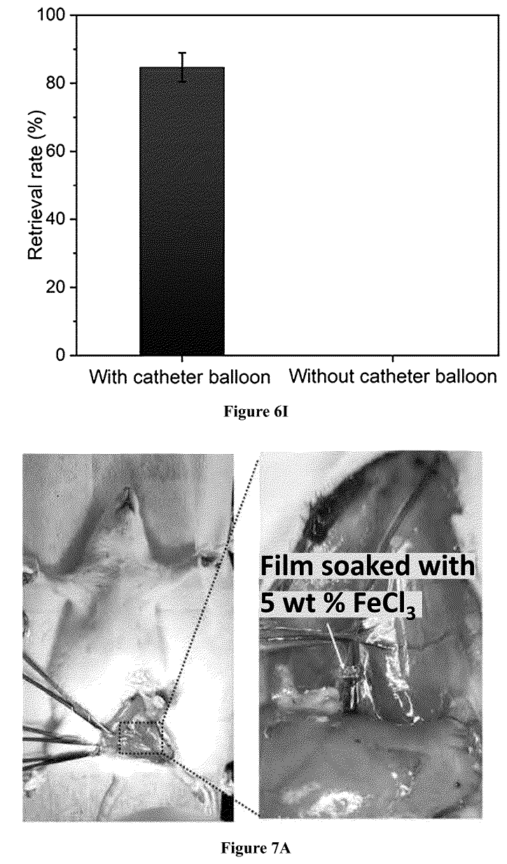

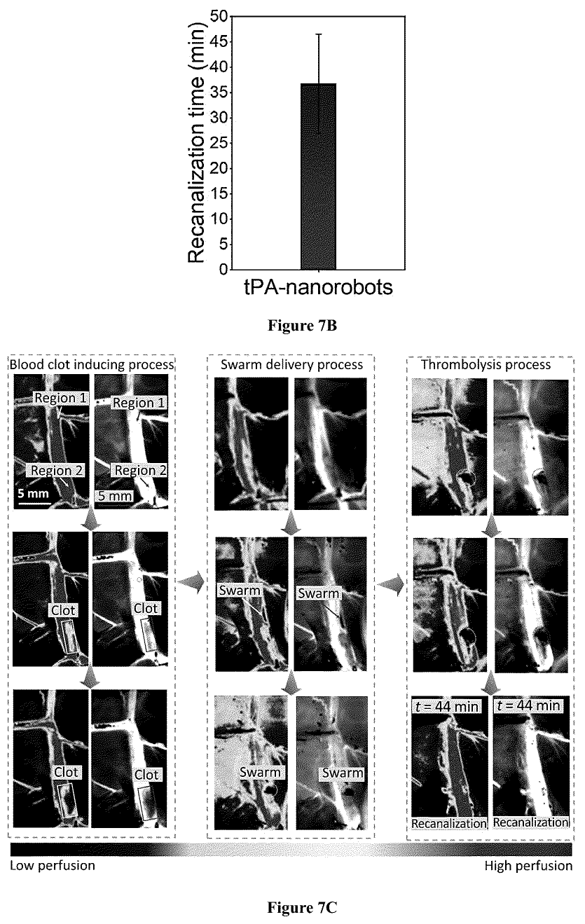

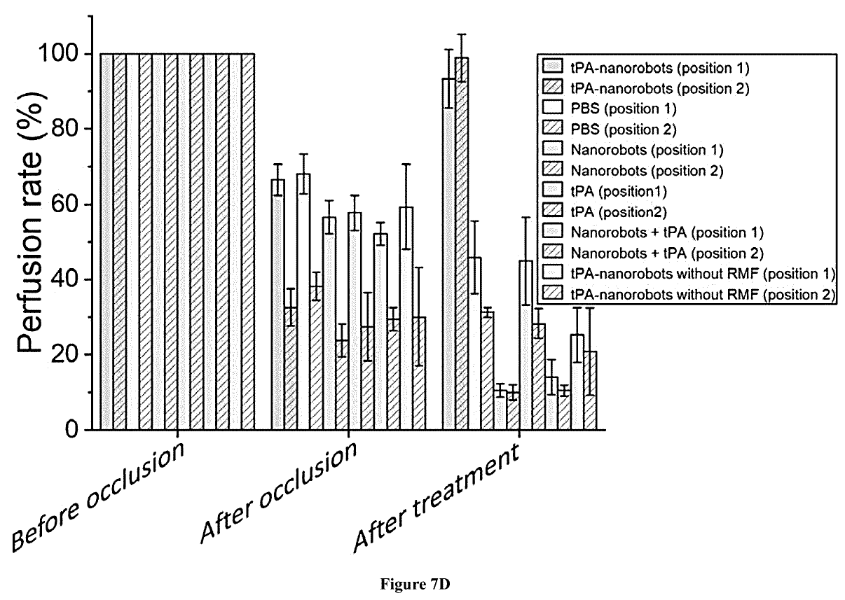

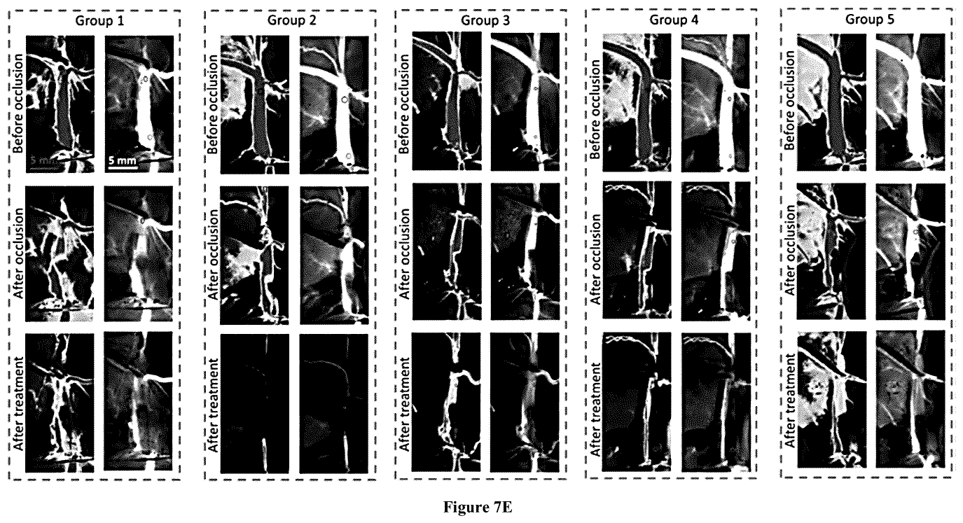

The thrombolysis effect of tPA-bots was verified in vivo for both the veins [using male SD rats (movie S6: In vivo thrombolysis with a rat model)] and arteries [using New Zealand white rabbit (movie S7: In vivo thrombolysis of the blood clots at the carotid artery with an inner diameter of 0.9 mm in an 8-week New Zealand white rabbit)]. FIG. 7A shows the procedure for inducing the blood clot in the femoral vein of the rat. Briefly, a fabric film after soaking with 5 wt % FeCl₃ solution covered the surface of the common femoral vein of the rat for 5 min. Then, the film was removed, allowing the blood clot formation and growth for 30 min. The blood clot gradually formed inside the common femoral vein with blood flow decreasing. The color of the occlusion region changed from red to blue in the pseudo-color pattern and white to black in the gray pattern, indicating the formation of a blood clot in the common femoral vein of the rat. After occlusion, tPA-nbots (0.5 mg) were injected by an indwelling needle from the epigastric vein. A RMF (magnetic field strength, 64.9 mT; frequency, 3 Hz) was applied for trapping and accumulation of tPA-nbots and further forming the microswarm. The femoral vein, once occluded, can be recanalized within 40 minutes after treatment with the tPA-labeled microswarm (FIG. 7B). As observed in the swarm delivery process of FIG. 7C, the swarm also exhibited imaging signals under LSBFIS, showing black color in pseudo-color pattern and gray pattern. Then, the microswarm was navigated toward the thrombus for targeted thrombolysis by using the RMF. The thrombolysis process in FIG. 7C demonstrated that the blood clot was gradually degraded by tPA-nbots, and recanalization of the occlusive femoral vein was realized after 44 min of treatment. After recanalization, the occlusive femoral vein nearly recovered to the original state as shown in the last image of FIG. 7C. The change in perfusion rate during the whole process was also evaluated. Two regions were selected as references to monitor the change in perfusion rate, as shown in the first image of FIG. 7C. FIG. 7D exhibited the results of perfusion rate change of six groups, and the perfusion rate was 100% before occlusion, referring to the original state of the femoral vein without any treatments. After occlusion, the perfusion rate decreased substantially, and around 30 to 40% decrease in region 1 and around 60 to 70% decrease in region 2 were observed for total six groups. The perfusion rates were increased to around 100% after being treated by a tPA-labeled microswarm with a RMF, implying that blood flow was fully restored. The average recanalization time was about 37 min using a tPA-labeled microswarm for in vivo thrombolysis (FIG. 7B), suggesting the excellent thrombolysis effect of tPA-nbots. Such a result also confirms that the mechanical interaction and chemical lysis endow the tPA-labeled microswarm with the best thrombolysis effect in vivo, which has been verified in vitro (FIGS. 4A to 4F). By comparison, the perfusion rates of nanorobots group with only mechanical interaction (10.5 and 10.0% for region 1 and region 2, respectively) and pure tPA group with only chemical lysis (44.9 and 28.3% for region 1 and region 2, respectively) substantially decreased after treatment, and the imaging of group 2 and group 3 in FIG. 7E showed that no obvious thrombus was removed. Besides, nanorobots group exhibited the highest decrease in perfusion rate after treatment, suggesting that the administration of nanorobots without tPA modification would aggravate the occlusion and further greatly decline the blood flow. Compared with nanorobots group, the perfusion rate of tPA-nbots without RMF was twofold higher than that of nanorobots group, reaching 25.3 and 20.8% for region 1 and region 2, respectively. Therefore, the ultralow dose of tPA modified on nanorobots could mitigate occlusion due to the localized thrombolysis in vivo. The images in FIG. 7E illustrated the different stages of control groups during the whole process, showing limited therapeutic effect compared with tPA-nbots group. In results, tPA-nbots with both mechanical interaction and chemical lysis had the best thrombolysis effect in vivo, and fast recanalization was achieved with nearly 100% recovery of perfusion rate after targeted therapy. These results verify the advanced ability for in vivo thrombus lysis by the proposed tPA-nbots.

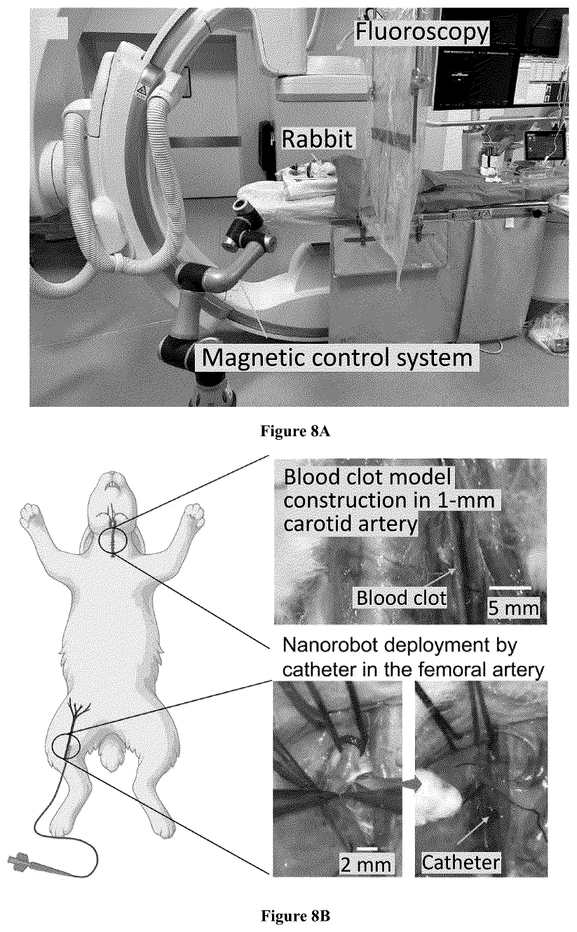

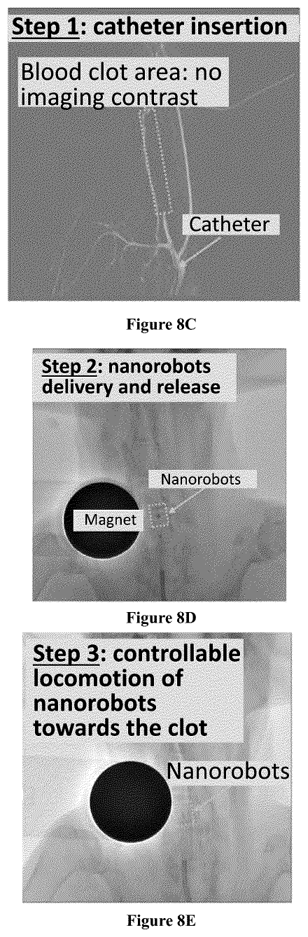

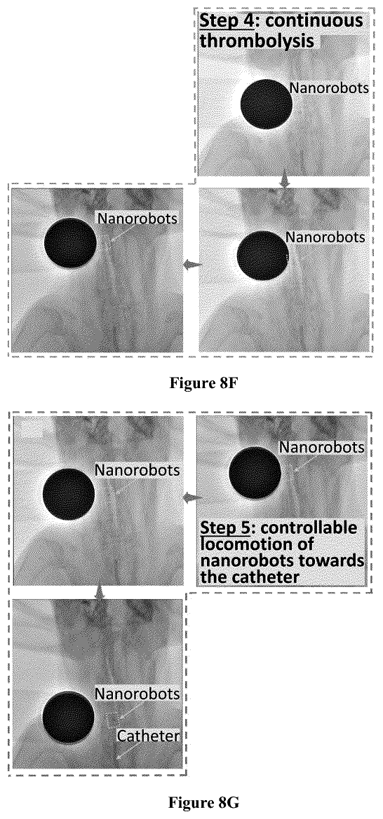

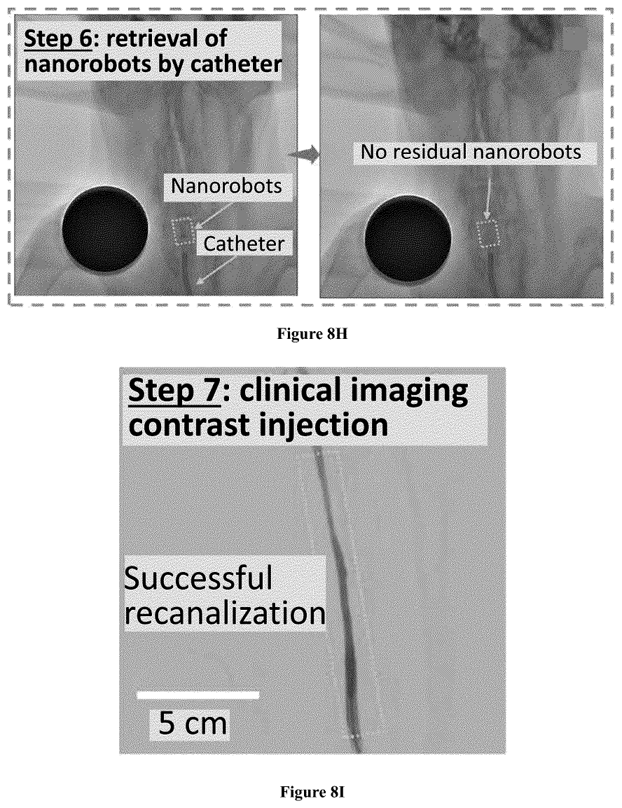

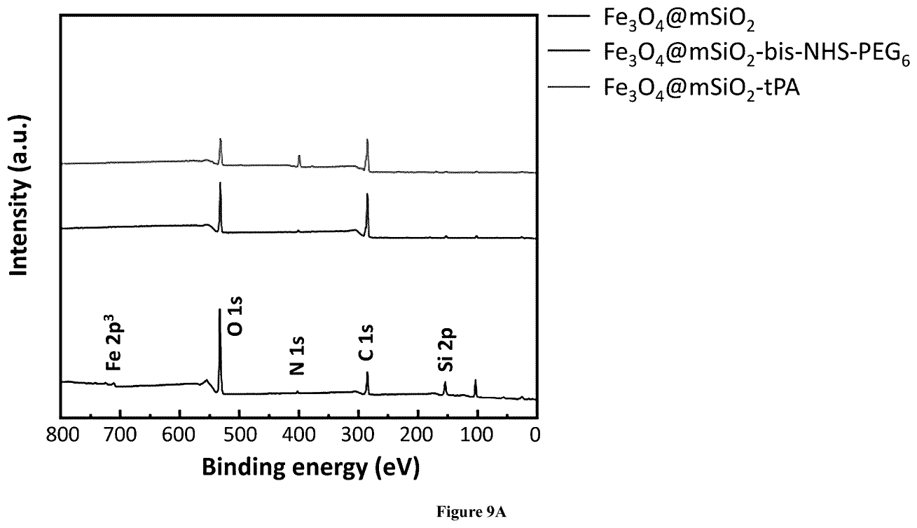

The in vivo thrombolysis of the blood clots was also verified at the carotid artery with an inner diameter of 0.9 mm (much smaller than the diameter of M3 segment) in an 8-week New Zealand white rabbit (movie S7). FIG. 8A shows the platform for in vivo thrombolysis of the blood clots at the carotid artery of New Zealand white rabbit. After the blood clot was induced inside the 0.9-mm carotid artery, a 1.1-mm catheter was threaded through the femoral artery in the groin and up to the limited position that can be reached in the carotid artery (FIG. 8B). The whole thrombolysis process was performed under real-time imaging with x-ray fluoroscopy and could be divided into the following steps. Step 1: catheter insertion (FIG. 8C). The catheter was threaded through the femoral artery and arrived at the carotid artery under the fluoroscopy. The iohexol was injected so that the blockage segment of the vessel could be clearly observed (vessels without imaging contrast). Step 2: nanorobots delivery and release (FIG. 8D). The mixture of tPA nanorobots and microrobots was injected into the vessel. The microscale robots (size: 500 μm) facilitate the much clearer tracking of the robot swarm in a dynamic and high-speed flow environment. Step 3: controllable locomotion of nanorobots toward the clot (FIG. 8E). The tPA-nbots were actuated with an external magnetic field (strength, 64.9 mT; frequency, 3 Hz) toward the blood clot. Step 4: continuous thrombolysis (FIG. 8F). The continuous thrombolysis was performed under a tumbling magnetic field (strength, 64.9 mT; frequency, 3 Hz; pitch angle 90°). The blood clot was scraped by the mechanical stirring and etched by biochemical lysis. Step 5: controllable locomotion of nanorobots toward the catheter (FIG. 8G). After the vessel was recanalized, the tPA-nbots were magnetically propelled back toward the catheter. Step 6: retrieval of nanorobots by catheter (FIG. 8H). As the tPA-nbots reached the terminal of the catheter, it was sucked into the catheter and the catheter was pulled out of the vessel to complete the vascular recanalization. Step 7: clinical imaging contrast injection (FIG. 8I). The iohexol was injected again and one can observe the recovered blood flow under fluoroscopic imaging. After the vessel was recanalized, the trapping capability of the tPA-nbots against the instantaneous blood flow was crucial and should be verified. As shown in FIGS. 33A to 33G, one can observe that, after the occlusion is removed, these nanorobots can be still trapped as well and can be visualized with the fluoroscopic imaging.

In Vivo Toxicity Evaluation of tPA-Nbots

Last, the in vivo safety profile of the tPA-labeled microswarms after intravenous injection was evaluated. A comprehensive blood analysis of mice injected with saline (200 μl, control group) and mice injected with tPA-nbots (200 μl) at 5 mg/ml, tPA-nbots at 10 mg/ml, and tPA-nbots at 25 mg/ml is conducted. The blood samples were collected on days 4 and 15, respectively. As shown in FIG. 34A, compared with control mice, the numbers of blood cells (red blood cells, white blood cells, and platelets) and levels of the biochemistry markers in the mice remained at normal levels with small fluctuations after injection of tPA-nbots with various concentrations. Similarly, an extended observation period of 15 days after injection (FIG. 35) revealed that, compared to control mice and the corresponding mice at day 4, those injected with tPA-nbots at various concentrations maintained blood cell counts and biochemical marker levels within normal ranges, with minor fluctuations. This comprehensive evaluation underscores the favorable in vivo safety profile of tPA-labeled microswarms across various concentrations over both short and long-term durations. In addition, no tissue damage was observed in the main organs, i.e., the heart, liver, spleen, lung, and kidney, from the histopathological evaluation after the tPA-nbots were injected into the mice for 1 week (FIG. 34B). The tPA-nbots show no obvious accumulation in the heart, liver, spleen, lung, and kidney. Furthermore, the biodistribution of the tPA-nbots at different time points in healthy rats was measured to study the biodistribution and accumulation of the magnetic particles in the major organs (FIG. 34C). The result suggested that the tPA-nbots show no obvious accumulation in the heart, liver, spleen, lung, and kidney and potentially have negligible side effects on the main organs, denoting the tPA-nbots are nontoxic and biocompatible. These results verified the in vivo therapeutic effect of tPA-labeled microswarm, and the materials showed negligible side effects to the organisms.

A catheter-assisted magnetic navigation system with x-ray fluoroscopic imaging tracking that enables the therapeutic intervention of blood clots by using tPA-grafted nanorobots (tPA-nbots) with the ability for rapid thrombus lysis and the ability to recapture the emitted nanobots. The system can remotely deploy and deliver the tPA-nbots to the thrombus site, inaccessible by conventional catheter methods, and achieve highly efficient thrombolysis through the synergy of mechanical and biochemical lysis. The in vitro removal of a blood clot can be commonly completed within 20 min, and the ex vivo removal of a blood clot in the human placenta can be commonly completed within 30 min. The tPA-nbots consist of a magnetic core, a mesoporous silica shell, and a grafted tPA outer layer with an anchoring capacity of about 15 mg/g. The anchored tPA on the nanorobots shows enzymatic activity comparable with that of the pure tPA. The therapeutic intervention of blood clots using the tPA-nbots is achieved by CMAFIS. For hard-to-reach vessels, such as M3/M4 segments and distal of the cerebral vessels, the developed CMAFIS can actuate and deliver the tPA-nbots while being able to track the movement by real-time x-ray fluoroscopic tracking. Furthermore, this method is endowed with the ability to navigate long distances and exhibits high targeting precision, rapid thrombus lysis, and, most notably, the ability to recapture the robots. The fabricated tPA-nbots were stored inside a catheter and deployed after the catheter reached the location in the vicinity of the thrombus. Then, the colloidal microswarm was steered toward the thrombus site using the magnetic control unit and fluoroscopic imaging for real-time navigation. The synergy of precisely controlled hydrodynamic flow and biochemical lysis contribute to the highly efficient dissolution of the blood clots. After the treatment process, the microswarm was navigated back to the site near the tip of the catheter. An extraction process was applied to collect the vast majority of the nanorobots with a high efficiency of about 80% of the initially deployed nanorobots. The proposed colloidal microswarm provides a promising robotic tool with high spatial precision to achieve enhanced thrombolysis with a low probability of side effects due to the recapturing of the nanobots and the low systemic drug load.

### Materials

FeCl₃·6H₂O [iron (III) chloride hexahydrate], CaCl₂) (calcium chloride), bis-NHS-PEG6, sodium citrate, TEOS, triethanolamine (TEA), FITC, dimethyl sulfoxide (DMSO), hexadecyltrimethylammonium chloride (CTAC), 1-octadecene, and EG were purchased from Aladdin Chemical Co. Ltd. PEG, C₂H₃NaO₂·3H₂O (sodium acetate trihydrate), and dopamine hydrochloride were purchased from Advanced Technology and Industrial Co. Ltd. (3-Aminopropyl)tricthoxysilane (APTES) was purchased from Sigma-Aldrich. All of the chemicals were used as received without further purification.

### Preparation of Magnetite Microspheres

The preparation of magnetite microspheres was conducted in accordance with the literature (46). Briefly, 1.35 g of FeCl₃·6H₂O was dissolved in 40 ml of EG by vigorous stirring to form an orange clear solution. Subsequently, 3.6 g of sodium acetate trihydrate and 1.0 g of PEG (M_{w}=50 000) were added, and the solution was treated ultrasonically for 30 min. Then, the mixture was sealed in a Teflon-lined stainless-steel autoclave and heated (200° C.) for 10 hours. Afterward, the autoclave was allowed to cool to room temperature. The black product was washed successively three times with ethanol and deionized (DI) water. Last, the magnetic particles were stored in DI water at a concentration of 20 mg/ml.

Synthesis of Fe₃O₄ωmSiO₂ Nanorobots

Fe₃O₄ωmSiO₂ nanorobots were prepared by a biphasic stratification method, applying TEOS as precursor, CTAC as template, TEA as catalyst, and 1-octadecene as emulsion agent. Briefly, 60 ml of water and the solution were gently stirred at an elevated temperature (60° C.), and 3 g of CTAC and 160 μl of TEA were added to the aqueous phase. Then, 2.5 ml of magnetite microsphere solution (see the previous paragraph) was added and dispersed in this solution. Subsequently, 20 ml of 1-octadecene solution with 10 v/v % TEOS was poured into the aqueous suspension and kept continuously stirring at 60° C. for 15 hours. The black products were harvested by a magnet and washed successively three times with DI water and ethanol.

### Surface Grafting of APTES

The Fe₃O₄ωmSiO₂ nanorobots were dispersed in a solution comprising 50 ml of ethanol and 1 ml of APTES and mechanically stirred in this solution for 12 hours. The APTES-functionalized nanorobots were collected by a magnet and washed three times with ethanol. Surface functionalization with bis-NHS-PEG6

Fe₃O₄ωmSiO₂ nanorobots (20 mg) grafted with APTES were washed three times with DMSO to remove the impurities and water. Bis-NHS-PEG6 was dissolved in DMSO with concentrations of 1 mg/ml, and triethylamine with a concentration of 1.7 mM was added. The bis-NHS-PEG6 solution (10 ml) was mixed with nanorobots and aged at room temperature for 2 hours. Last, the nanorobots were separated with a magnet and washed three times with DMSO.

Fabrication of Fe₃O₄ωmSiO₂-tPA Nanorobots

The functionalized nanorobots were washed three times with water. Phosphate buffer (pH 7.2) was prepared with a phosphate concentration of 0.1 M. tPA was dissolved in the buffer (20 ml) to yield a final concentration of 2.5 mg/ml. The cross-linked nanorobots were added into the tPA aqueous solution, and the mixture was stirred for 2 hours at room temperature. Afterward, the obtained Fe₃O₄ωmSiO₂-tPA nanorobots were collected with a magnet, dispersed in DI water, and stored in a fridge at 4° C.

Fabrication of Fluorescent Fe₃O₄ωmSiO₂-tPA Nanorobots

The abovementioned Fe₃O₄ωmSiO₂-tPA nanorobots were dispersed in 0.1 M sodium carbonate buffer adjusted to pH 9. Concurrently, an FITC solution with a concentration of 1 mg/ml in dry DMSO was prepared in a dark environment. FITC solution (100 μl) was added to the nanorobot buffer solution and gently shaken for 8 hours in a dark environment. Afterward, the product was washed three times with DI water and stored in a fridge at 4° C.

Quantification of Surface Grafting Density of tPA on the Fe₃O₄ωmSiO₂ Nanorobots

The tPA grafting density was assessed by a fluorescence plate reader at an excitation wavelength of 494 nm and an emission wavelength of 518 nm. Briefly, an excess amount of FITC-labeled tPA with a concentration of 0.1 mg/ml in Hepes buffer was allowed to react with Fe₃O₄ωmSiO₂ nanorobots grafted with APTS for 2 hours. Then, the magnetic nanorobots were separated with a magnet and washed three times with DI water. After that, the magnetic nanorobots were dispersed in DI water and analyzed by a fluorescence plate reader to determine the fluorescence intensity. A standard fluorescence intensity curve at various dose of FITC-labeled tPA was measured. The fluorescence intensity of the prepared magnetic nanorobots was compared with the measured curve between the fluorescence intensity, and the dose of FITC-labeled tPA to determine the grafting density of the tPA on the Fe₃O₄ωmSiO₂.

### Swarming Motion Generation and Analysis

The swarming motion generation process and navigation speed were investigated on both flat and curved surfaces. For the flat surface, a glass tank with a diameter of 45 mm was used. Plasma (2 ml) collected from pig blood was added into the glass tank, and 0.2 mg of tPA-nbot was added by a pipette. Then, a magnetic field was applied immediately, with different magnetic field strengths (7.5, 10.6, 15.1, 22.4, 37.2, 64.9, and 121.5 mT) and frequencies (2, 3, 4, 5, 6, 7, 8, 9, and 10 Hz) to study the generation area and time of colloidal microswarm. The navigation speed of colloidal microswarm on a flat surface was also explored, varying the different magnetic field strengths and frequencies. For the curved surface, a silicon tube with an inner diameter of 1.5 mm was filled with whole pig blood for swarming motion analysis and ultrasound imaging. The B-mode and Doppler signals can be observed by ultrasound imaging. Similarly, the generation time, generation area, and navigation speed of the colloidal microswarm were investigated with different magnetic field strengths (7.5, 10.6, 15.1, 22.4, 37.2, 64.9, and 121.5 mT) and frequencies (2, 3, 4, 5, 6, 7, 8, 9, and 10 Hz). The magnetic field strength was measured by using a Gaussmeter (HT201) purchased from Hengtong magnetoelectricity Co. Ltd.

### Collection of Human Placenta and Patient Eligibility

The human placenta obtained from the Prince of Wales Hospital was used for ex vivo thrombolysis analysis. The collection of human placentas was approved and overseen by The Joint Chinese University of Hong Kong-New Territories East Cluster Clinical Research Ethics Committee (ref. no. 2020.384). All enrolled patients provided a written informed consent.

### Generation of Blood Clots in Channels

Fresh pig blood was obtained from the Prince of Wales Hospital and stored at 10 v/v % in sodium citrate solution (3.2 wt %) in a fridge at 4° C. To form the blood clot, 20 μl of 0.5 M CaCl₂) solution was added to 1 ml of fresh pig blood, and the mixture was shaken for 1 min. Subsequently, a desired volume of blood was injected into a channel. Then, the channel was aged to form the blood clot. Afterward, saline solution was injected into the channel from both sides of the blood clot.

### In Vitro Thrombolysis

In vitro thrombolysis using nanorobots was performed in the glass tube, plexiglass with 3D channels, and phantom. All the blood clots were induced by the same procedure as we mentioned before. The temperature control methods were different for the glass tube, plexiglass, and phantom. For blood clots in the glass tube, the glass tube was inserted into a cuboidal channel filled with water of ˜37° C. and sealed by hot-melt adhesive. For thrombus in the plexiglass with 3D channels, a heating pad was used to control the temperature of the plexiglass at around 37° C., and a thermal meter was used to calibrate the temperature of the plexiglass. For thrombus in the phantom, a heating pad and infrared light were used to keep the temperature of the phantom at around 37° C., and the 4× diluted blood was incubated in the water bath with ˜37° C. temperature before pumping to the phantom. Here, the thrombolysis rates were obtained by using the following equation

$$Thrombolysis rate = \frac{Thrombus volume}{Thrombolysis time} = \frac{l π {r}^{2}}{t} \qquad Formula ( 5 )$$

therein, I refers to the length of the thrombus, r refers to the radius of the thrombus, and t refers to the thrombolysis time. Here, the thrombus was regarded as a cylinder.

### In Vitro Demonstration of Thrombolysis in Phantom and Retrieval of Nanorobots

Here, a phantom with multibranches was used as a model. One milliliter of fresh pig blood mixed with 20 μl of 0.5 M CaCl₂) solution was added to one branch of the phantom; after aging, a blood clot with the length of 5 mm and diameter of 2 mm was successfully induced. The catheter was used for the delivery and retrieval process. Step 1: The catheter was inserted into the phantom, where the catheter tip was at the site with a distance of 40 mm to the blood clot, and the blood flow was temporally blocked. Step 2: tPA-nanorobots (1.31 mg) were released from the catheter and further formed the swarming by a RMF (magnetic field strength, 37.5 mT; frequency, 3 Hz). Step 3: The tPA-nanorobot microswarm was guided toward to the blood clot and performed targeted thrombolysis. Step 4: After removing the blood clot, tPA-nanorobot microswarm moved to the retrieval site near the catheter tip, and retrieval of tPA-nanorobots was realized by catheter suction.

### Ex Vivo Demonstration of Thrombolysis in Human Placenta and Retrieval of Nanorobots

The human placenta obtained from the Prince of Wales Hospital was used as an ex vivo model for thrombolysis and retrieval of nanorobots. First, the blood clot was induced in one branch of the blood vessel. Twenty microliters of 0.5 M CaCl₂) solution was added to 1 ml of fresh pig blood, and, then, the blood was injected into the branch of the vessel. After that, the whole placenta was transferred to an oven with a temperature of 37° C. for incubation of the blood clot. Once the blood clot was formed, the placenta was transferred onto a heating pad and irradiated by infrared light to control the temperature at around 37° C. The whole procedure of thrombolysis and retrieval of nanorobots in the placenta consisted of three stages. Stage 1: The catheter was inserted into the placenta at the vessel that is in close vicinity to the vessel with the blood clot, and tPA-nbots were released from the catheter with an injection speed of 0.05 ml/s and trapped by the magnetic field, while real-time tracking by fluoroscopy was conducted. In addition, the catheter balloon was inflated to block the blood flow temporarily. Stage 2: The colloidal microswarm was magnetically navigated toward the blood clot for thrombolysis while real-time tracking by fluoroscopy was performed. Stage 3: After the successful removal of the blood clot, the nanorobots were magnetically guided back to the tip of the catheter, and retrieval of nanorobots was realized by catheter suction. Then, the catheter was extracted from the placenta.

### In Vivo Thrombolysis

The animal experiments were approved by the Animal Experimentation Ethics Committee (no. 20-148-CRF) in the Chinese University of Hong Kong. Male SD rats weighing 300 to 350 g were kept under 12-hour light/12-hour dark cycle and provided with free access to food and water for using in this study. First, a thrombus model was constructed in the femoral vein of the rat. A fabric film with width of 2 mm was soaked with 5 wt % FeCl₃ solution, and, then, a fabric film was covered on the surface of the common femoral vein for 5 min. After incubation and growth for 30 min, the blood clot was successfully formed. Adult rats were randomly divided into six groups: tPA-nanorobot group, phosphate-buffered saline (PBS) group, nanorobot group, tPA group, nanorobot+tPA group, and tPA-nanorobot without RMF group. For tPA-nanorobots group, 0.5 mg of tPA-nanorobots with concentration of 2 mg/ml was injected into the femoral vein by an indwelling needle from epigastric vein. A RMF was applied with magnetic field strength of 64.9 mT and frequency of 3 Hz. Then, tPA-nanorobots locomoted to the blood clot site for thrombolysis under magnetic field control. During the thrombolysis process, an infrared light was used to keep the rat warm. For the PBS group, 0.25 ml of PBS was injected into the femoral vein as the blank control, other experimental conditions were the same as that in the tPA-nanorobot group. For the nanorobot group, 0.5 mg of Fe₃O₄ωSiO2 nanoparticles was injected into the femoral vein and guided toward the blood clot with RMF for treatment of 1 hour. For the tPA group, 0.25 ml of PBS with 7.5 μg of tPA was injected into the femoral vein as the control experiment with treatment time of 1 hour. For the nanorobot+tPA group, 0.5 mg of Fe₃O₄ωSiO2 nanoparticles and 7.5 μg of tPA were administrated to confirm the thrombolysis effect of Fe₃O₄ωSiO2 nanoparticles with separated tPA. For the tPA-nanorobot without RMF group, 0.5 mg of tPA-nanorobots was delivered to the thrombus, and a static magnetic field was applied to trap the swarm showing no rotation motion. After treatment for 1 hour, the perfusion rates of the experimental group and the control groups were calculated for quantified analysis. On the two sides of the fabric film, position 1 and position 2 as illustrated in FIG. 7C were chosen to monitor the change of blood perfusion by LSBFIS. Before occlusion, the perfusion rate was defined as 100%. After occlusion or treatment, the perfusion rates were also calculated by following equation

$$Perfusion rate = \frac{Perfusion unit}{Original perfusion unit} * 100 \% \qquad Formula ( 6 )$$

### Supplementary Experimental Section

### In Vitro Retrieval of Nanorobots-tPA

The retrieval efficiency of nanorobots-tPA was studied in the in vitro model filled with PBS solution. A low-profile PTA balloon dilatation catheter (Advance 35LP) purchased from Cook Incorporated with an inner diameter of 1 mm and an overall length of ˜142 cm was deployed for nanorobots delivery and retrieval process. A silicone tube with a diameter of 2.5 mm was used as the artificial blood vessel model. Firstly, the influence of injection dose, nanorobots concentration, injection speed, distance between catheter tip and magnet central, magnetic field strength, as well as the flow speed on trapping efficiency of nanorobots is considered. In order to precisely control the dose, concentration, and injection speed, the catheter was filled with nanorobots solution before the injection process and then injected the nanorobots solution into the tube as demanded. For trapping percentage, the following equations were used for calculation:

$$Trapping percentage = \frac{Trapped weight}{Injected weight} \qquad Formula ( 7 )$$

$$Loss weight = Injected weight - Trapped weight \qquad Formula ( 8 )$$

$$Formula ( 9 )$$

$$Overall loss weight = Loss weight in the catheter + Loss weight$$

Trapped weight refers to the weight of nanoparticles trapped by the rotating magnetic field, injected weight refers to the overall weight of nanoparticles injected into the tube, including nanoparticles trapped/non-trapped by the rotating magnetic field, loss weight refers to the weight of nanoparticles injected to the tube but not trapped by rotating magnetic field, overall loss weight consists of two parts, first part is the loss weight of nanoparticles in the catheter, other part is the loss weight of nanoparticles injected to the tube but not trapped by rotating magnetic field.

Detailed experimental information is as follows. For group of injection dose: Injection speed: 0.05 mL/s, Dose: 1, 2, 3, 4, 5 mg, Concentration: 2 mg/mL, Frequency: 3 Hz, Magnetic field strength: 64.9 mT, Distance between catheter tip and magnet center: 2.0 cm; For group of nanorobots concentration: Injection speed: 0.05 mL/s, Dose: 3 mg, Concentration: 0.5, 1, 2, 3, 4, 5, 6 mg/mL, Frequency: 3 Hz, Magnetic field strength: 64.9 mT, Distance between catheter tip and magnet center: 2.0 cm; For group of injection speed: Injection speed: 0.025, 0.05, 0.1, 0.2, 0.2, 0.3 mL/s, Dose: 3 mg, Concentration: 2 mg/mL, Frequency: 3 Hz, Magnetic field strength: 64.9 mT, Distance between catheter tip and magnet center: 2.0 cm; For group of magnetic field strength: Injection speed: 0.05 mL/s, Dose: 3 mg, Concentration: 2 mg/mL, Frequency: 3 Hz, Magnetic field strength: 121.5, 64.9, 37.2, 22.4 mT, Distance between catheter tip and magnet center: 2.0 cm; For group of distance: Injection speed: 0.05 mL/s, Dose: 3 mg, Concentration: 2 mg/mL, Frequency: 3 Hz, Magnetic field strength: 64.9 mT, Distance between catheter tip and magnet center: 1.0, 2.0, 3.0, 4.0, 5.0 cm; For group of flow speed: Injection speed: 0.05 mL/s, Dose: 3 mg, Concentration: 2 mg/mL, Frequency: 3 Hz, Magnetic field strength: 64.9 mT, Distance between catheter tip and magnet center: 2.0 cm, flow speed: 1, 3, 5, 7, 9, 11, 13, 15, 17, 19 cm/s.

Then, the retrieval efficiency of nanorobots was confirmed using the same model with additional procedures. Step 1: The catheter was inserted into the tube, and nanorobots were released from the catheter and then trapped by the magnetic field; Step 2: After the generation of colloidal microswarm, they performed the downstream locomotion for a distance of 50 mm controlled by the magnetic field, and then moved against the flow until reaching the catheter tip; Step 3: The retrieval process was conducted by catheter suction. Here, injection speed: 0.05 mL/s, dose: 3 mg, concentration: 2 mg/mL, frequency: 3 Hz, magnetic field strength: 64.9 mT, distance between catheter tip and magnet center: 2.0 cm, locomotion speed: ˜ 0.5 mm/s, extraction speed: 0.25 mL/s; flow speed: 0, 1, 3, 5, 7, 9, 11, 13 cm/s.

For retrieval percentage, the following equations were used for calculation:

$$Retrieval percentage = \frac{Retrieval weight}{Injected weight} \qquad Formula ( 10 )$$

$$Retrieval weight = Overall weight - Weight in the catheter \qquad Formula ( 11 )$$

Retrieval weight refers to the weight of nanoparticles retraveled from the tube after procedures, injected weight refers to the weight of nanoparticles injected into the tube, including nanoparticles trapped/non-trapped by the rotating magnetic field, and overall weight refers to the weight of all nanoparticles collected from tube and catheter; therefore, retrieval weight of nanoparticles can be obtained by overall weight subtracting the remained weight of nanoparticles in the catheter. All samples were collected from the catheter and/or tube, followed by drying and weighing.

### Expanded Information on Collection of Human Placenta and Patient Eligibility

The human placenta obtained from the Prince of Wales Hospital was used for ex vivo thrombolysis analysis. The collection of human placentas was approved and overseen by The Joint Chinese University of Hong Kong-New Territories East Cluster Clinical Research Ethics Committee (The Joint CUHK-NTEC CREC) (Ref. No. 2020.384). All enrolled patients provided written informed consent. The human placenta was donated by pregnant women in collaboration with the Department of Obstetrics and Gynaecology (CUHK). Eligible patients were (i) healthy pregnant women at 20-45 years of age of any ethnic origin, giving childbirth with Caesarean sections after 37-42 weeks of gestation; (ii) singleton pregnancy; (iii) healthy as determined by laboratory results, physical exam and medical history; and (iv) participant able to give voluntary, written, informed consent to participate in the study. Patients were excluded if they have abnormal prenatal development (e.g. intrauterine growth restriction), hypercholesterolemia, family history of stroke or vascular diseases, diabetes and gestational diabetes and cancer. On the day of labour, the placentas were collected by the surgeons and delivered on ice to the laboratory immediately after labouring. The placentas were completely soaked in a solution with 4% sodium citrate and 0.9% sodium chloride in order to prevent the residual blood in the blood vessels from occlusion. Pre-treatment of the placenta was processed before experiments. The placenta membranes were removed firstly; then, the whole placentas were washed with the saline until there was no obvious residual blood. To completely remove the blood in the vessels, the saline was injected into the blood vessels repeatedly until the blood vessels were clear. After that, the whole placentas were immersed in a solution with 4% sodium citrate and 0.9% sodium chloride and stored in a refrigerator with a temperature of 4° C. before use.

### Ex Vivo Retrieval of Nanorobots-tPA

The retrieval efficiency of nanorobots was also investigated in an ex vivo model. During the experiment, the pig blood was pumped into the placenta blood vessels through the umbilical cord with a silicone tube fixed inside by the surgical suture. In order to allow complete blood flow in the placenta, the silicone tubes were inserted into the distal blood vessels of each branch and fixed using the surgical suture, which allows the blood to flow back to the beaker. The retrieval efficiency of nanorobots from the placenta was studied with the assistance of the catheter. The procedures are similar to in vitro model. Step 1: The catheter was inserted into the blood vessel, and nanorobots were released from the catheter, then trapped by the magnetic field; Step 2: After the generation of swarming motion, the colloidal microswarm locomoted for a certain distance and then returned to the catheter tip; Step 3: The retrieval process was conducted by catheter suction. The influence of dose (1, 2, 3, 4, 5 mg), magnetic field strength (121.5, 64.9, 37.2, 22.4 mT), locomotion distance (6, 8, 10, 12, 14 cm), flow speed (0.5-12.9 cm/s), and blood vessel diameter (1.4-5.5 mm) on the retrieval rate were quantitatively investigated. Other parameters are fixed, injection speed: 0.05 mL/s, concentration: 2 mg/mL, frequency: 3 Hz, distance between catheter tip and magnet center: ˜15 mm. To quantitively analyze the weight of lost nanoparticles, the silicone tubes connected to the end of placenta blood vessels were placed on a big magnet in order to trap the lost nanoparticles. The following equations were used for calculation:

$$Retrieval percentage = \frac{Retrieval weight}{Injected weight} \qquad Formula ( 12 )$$

$$Retrieval weight = Overall weight - Weight in the catheter \qquad Formula ( 13 )$$

$$Lost percentage = \frac{Weight trapped by magnetic field}{Injected weight} \qquad Formula ( 14 )$$

Retrieval weight refers to the weight of nanoparticles retraveled from the placenta after procedures, injected weight refers to the weight of nanoparticles injected into the placenta, including nanoparticles trapped/non-trapped by the rotating magnetic field, overall weight refers to the weight of all nanoparticles collected from placenta and catheter; therefore, retrieval weight of nanoparticles from the placenta can be obtained by overall weight subtracting the remained weight of nanoparticles in the catheter. For the lost percentage of nanoparticles, the weight of nanoparticles lost due to the blood flow during the whole process was mainly considered. The average of three experiments was taken for calculation. All samples were collected and followed by drying and weighing for calculation.
Biosafety of tPA-Nanorobot

### Cell Viability Assay

To assess the biosafety of microswarm, mesenchymal stem cells (MSCs) and human umbilical vein epithelial cells (HUVECs) at a density of 5000 cells/well were seeded in 96-well plates and then cultured in 100 μL of Eagle's Minimum Essential Medium (DMEM) containing 10% fetal bovine serum for 12 h. The medium was then discarded. 100 μL of fresh medium containing different concentrations of tPA-nanorobots (200, 400, 600, 800, 1000 μg/mL) was added to the HUVECs and MSCs. Subsequently, these different samples were co-cultured with cells for 24 h. The MTS assay quantified the viability of the cells. 10 μL MTS solution was added to each well, followed by a two-hour incubation. The microrobots were then concentrated on the bottom with a permanent magnet, and the supernatant was transferred to a new 96-well plate. The absorbance was detected at 490 nm using a microplate reader. All experiments were repeated three times. The microrobots were nontoxic to both MSCs and HUVECs, with the concentration reaching 1000 μg/mL, indicating good biocompatibility.

### Accumulation of Particles in Main Organs

Sample preparation: The animal experiments were approved by the Animal Experimentation Ethics Committee (AEEC No. 20-148-CRF) in the Chinese University of Hong Kong. Male Sprague-Dawley (SD) rats weighing 300-350 g were kept under 12 h-light/12 h-dark cycle and provided with free access to food and water for use in this study. 24 rats were randomly divided into 4 groups, and the control group was injected with 1 mL of PBS via tail vein injection, other 3 groups were injected with different doses of nanoparticles, 0.5 mg, 1 mg, 2 mg, respectively. After 1 day and 1 week, those rats were sacrificed, and the main organs, including heart, liver, spleen, lung, kidney, and brain were collected and stored in −80° refrigerator for further use.

Organ digestion: A digestion machine was employed to totally degrade the tissue. The heart, liver, spleen, lung, kidney, and brain were respectively added into 6 digestion vessels with 30 mL of 5 wt. % nitric acid. Then the vessel was placed into the digestion machine for 10 min, and then the solution was collected for further analysis.

ICP-MS test: Before sample testing, a standard curve was obtained by using the iron solution with concentrations of 0, 20, 40, 60, 80, 100 ppb, respectively. Then 10 μL of digestion solution was added to a plate tube with 10 mL of 3 wt. % nitric acid for ICP-MS test. After test, Fe 57 Helium KED (cps) was acquired and compared with the standard curve to obtain the iron content in the main organs.

### Characterization and Measurements

Young's modulus of the blood clot was measured by a micromechanical system (MACH-ITM, BIOMOMENTUM). Firstly, blood clots with different stiffness were induced in a plate with diameter of 10 mm and height of 2 mm. Different incubation times (15 min, 30 min, 60 min) were applied to blood clots to enable different stiffness. After incubation, blood clots were transferred to the sample holder to test the compression modulus. SEM images were obtained using a JEOL JSM-7800F scanning electron microscope. The specimens were sputtered with gold before SEM imaging. TEM images were captured using the JEOL Model JEM-2011 System. The ultrasound (US) imaging was conducted by a US imaging setup (Terason t3200, Teratech Corporation, USA). The blood flow was observed by the laser speckle blood flow imaging system (RWD RFLSI III). The fluoroscopy (SIEMENS Aritis Zeego) was used for real-time imaging of magnetic microswarm for targeted thrombolysis in the placenta. Organ digestion was conducted in a digestion machine (discover SP-D 80 automated microwave digestion system), and iron accumulation analysis was investigated by ICP-MS (PerkinElmer, NexlON 1000 G ICP Mass spectrometer).

Motion Control of the tPA-Nbots Swarm in Vessel

The swarm locomotion inside vessel is generally the swarming on a curved surface along the axis direction. The swarm generation of the tPA-nbots is triggered by a rotating magnetic field in the experimental plane. Actuated by the external magnetic fields, adjacent magnetized nanorobots form chains because of the dipole attractive forces, and multiple local vortices are generated by the rotation of the nanorobot chains (FIG. 15A). For a nanorobot chain with N particles, the swarm is driven by the magnetic torque (τ_{cmag}), which is exerted on the chain center. (39,40)

$${τ}_{cmag} = 2 {\sum }_{i = 1}^{N / 2} {F}_{i}^{t} {r}_{i} = \frac{3 {μ}_{0} {m}_{p}^{2}}{2 π} sin ( 2 θ ) {\sum }_{i = 1}^{N / 2} [ {r}_{i} {\sum }_{j = 1 , j \neq i}^{N} \frac{1}{{r}_{i , j}^{4}} ] \qquad Formula ( 15 )$$

Where Fᵢ^{t} and rᵢ represent the magnetic force perpendicular to the chain line generated by the i^{th} nanorobot, and the distance between the nanorobot and the chain center, respectively. Concomitantly, the chain is decelerated by the fluidic drag torque (τ_{cdrag}), which exhibits the opposite direction to τ_{cmag}. (41)

$${τ}_{cmag} = \frac{8 π {R}_{s}^{3}}{3} \frac{{N}^{3}}{ln ( N / 2 ) + 2.4 / N} η ω \qquad Formula ( 16 )$$

Here η is the fluidic dynamic viscosity. All single chain in a swarm shows the same direction in τ_{cmag}. The interactive magnetic force of a couple of nanorobot chains (Fᵢ) can be expressed as (42)

$${F}_{i} = \frac{3 {μ}_{0} {m}_{i} {m}_{j}}{4 π {r}_{i , j}^{4}} [ ( 1 - 5 {( m \cdot {r}_{i , j} )}^{2} ) {r}_{i , j} + 2 ( m \cdot {r}_{i , j} ) m ] \qquad Formula ( 17 )$$

Where mᵢ and mⱼ represent the magnetic moment norms of the adjacent nanorobot chains whose direction is m. The direction of m can be controlled to generate either attractive or repulsive forces between dissimilar chains, resulting in the assembly (gathering) and disassembly (spreading) of the magnetic particle swarm pattern, as showed by the optical images in FIG. 15B.

The generation time of the swarm, as well as the generation area can be controlled by adjusting the input frequency. When the magnetic strength is above 22.5 mT, the microswarm is in the stable state. The generation time decreases with the increase of frequency. On the contrary, the generation area will become larger. With decrease of magnetic strength, the “step-out” frequency is reduced (FIGS. 15C and 15D). The as-generated microswarms can perform on-demand locomotion along pre-designed trajectories, as demonstrated in FIG. 15E. The locomotion on both, flat surface and curved surface, were verified and quantitatively investigated in FIGS. 15F to 15M. For swarm locomotion on a flat surface, the input frequency and field strength significantly affect the collective motion of the nanorobots. FIGS. 15G and 15H present the phase diagrams, which show the relationships between the swarm patterns and the input frequency and field strength, respectively. For a certain frequency and field strength, the stable swarm pattern (FIG. 151) can be generated at a relatively low navigation speed. The increase of the locomotion speed will cause instability of the swarm pattern (transformation from stable pattern to unstable pattern, FIG. 151), resulting in partial lagging of the nanorobot swarm. As the frequency is increased, the maximum locomotion speed of a stable swarm pattern increases accordingly and finally reaches a steady state. As the field strength is increased, the maximum locomotion speed of a stable swarm pattern is increased up to approx. 300 μm/s at 37.2 mT, and it decreases once a magnetic field strength of 64.9 mT is overcome. For swarm locomotion on a curved vessel, which is similar to an M3/M4 blood vessel, the stable swarm patterns (FIGS. 15K to 15M) are easily generated at a relatively low swarm locomotion speed. The maximum locomotion speed of a stable swarm pattern increases with the input frequency up to a frequency of 7 Hz. At frequencies exceeding 7 Hz, the max. velocity dwindles sharply. Furthermore, the maximum locomotion speed increases monotonically with the field strength and a max. velocity of around 1.4 mm/s is observed at 121.5 mT.

### REFERENCES

1. L. Xiong, G. Tian, H. Leung, Y. O. Y. Soo, X. Chen, V. H. L. Ip, V. C. T. Mok, W. C. W. Chu, K. S. Wong, T. W. H. Leung, Autonomic dysfunction predicts clinical outcomes after acute ischemic stroke: A prospective observational study. Stroke 49, 215-218 (2018).
2. Y. Kim, E. Genevriere, P. Harker, J. Choc, M. Balicki, R. W. Regenhardt, J. E. Vranic, A. A. Dmytriw, A. B. Patel, X. Zhao, Telerobotic neurovascular interventions with magnetic manipulation. Sci. Robot. 7, cabg9907 (2022).
3. Y. Kim, G. A. Parada, S. Liu, X. Zhao, Ferromagnetic soft continuum robots. Sci. Robot. 4, caax 7329 (2019).
4. N. Korin, M. Kanapathipillai, B. D. Mattthews, M. Crescente, A. Brill, T. Mammoto, K. Ghosh, S. Jurek, S. A. Bencherif, D. Bhatta, A. U. Coskun, C. L. Feldman, D. D. Wagner, D. E. Ingber, Shear-activated nanotherapeutics for drug targeting to obstructed blood vessels. Science 337, 738-742 (2012).
5. A. Y. Prilepskii, A. F. Fakhardo, A. S. Drozdov, V. V. Vinogradov, I. P. Dudanov, A. A. Shtil, P. P. Bel′tyukov, A. M. Shibeko, E. M. Koltsova, D. Y. Nechipurenko, V. V. Vinogradov, Urokinase-conjugated magnetite nanoparticles as a promising drug delivery system for targeted thrombolysis: Synthesis and preclinical evaluation. ACS Appl. Mater. Interfaces 10, 36764-36775 (2018).
6. J. Hu, S. Huang, L. Zhu, W. Huang, Y. Zhao, K. Jin, Q. ZhuGe, Tissue plasminogen activator-porous magnetic microrods for targeted thrombolytic therapy after ischemic stroke. ACS Appl. Mater. Interfaces 10, 32988-32997 (2018).
7. H. B. van der Worp, J. van Gijn, Acute ischemic stroke. N. Engl. J. Med. 357, 572-579 (2007).
8. H. P. Adams, G. del Zoppo, M. J. Alberts, D. L. Bhatt, L. Brass, A. Furlan, R. L. Grubb, R. T. Higashida, E. C. Jauch, C. Kidwell, P. D. Lyden, L. B. Morgenstern, A. I. Qureshi, R. H. Rosenwasser, P. A. Scott, E. F. M. Wijdicks, Guidelines for the early management of adults with ischemic stroke: A guideline from the American Heart Association/American Stroke Association Stroke Council, Clinical Cardiology Council, Cardiovascular Radiology and Intervention Council, and the atherosclerotic peripheral vascular disease and quality of care outcomes in Research Interdisciplinary Working Groups: The American Academy of Neurology affirms the value of this guideline as an educational tool for neurologists. Circulation 22, 115 (2007).
9. G. J. del Zoppo, J. L. Saver, E. C. Jauch, H. P. Adams, Expansion of the time window for treatment of acute ischemic stroke with intravenous tissue plasminogen activator: A science advisory from the American Heart Association/American Stroke Association. Stroke 40, 2945-2948 (2009).
10. M. Wan, T. Li, H. Chen, C. Mao, J. Shen, Biosafety, functionalities, and applications of biomedical micro/nanomotors. Angew. Chem. Int. Ed. Engl. 60, 13158-13176 (2020).
11. B. Wang, K. Kostarelos, B. J. Nelson, L. Zhang, Trends in micro-/nanorobotics: materials development, actuation, localization, and system integration for biomedical applications. Adv. Mater. 33, e2002047 (2021).
12. H. Ceylan, I. C. Yasa, U. Kilic, W. Hu, M. Sitti, Translational prospects of untethered medical microrobots. Prog. Biomed. Eng. 1, 012002 (2019).
13. H. Choi, J. Yi, S. H. Cho, S. K. Hahn, Multifunctional micro/nanomotors as an emerging platform for smart healthcare applications. Biomaterials 279, 121201 (2021).
14. P. Wrede, O. Degtyaruk, S. Kumar Kalva, X. L. Deán-Ben, U. Bozuyuk, A. Aghakhani, B. Akolpoglu, M. Sitti, D. Razansky, Real-time 3D optoacoustic tracking of cell-sized magnetic microrobots circulating in the mouse brain vasculature. Sci. Adv. 8, eabm9132 (2022).
15. M. Xie, W. Zhang, C. Fan, C. Wu, Q. Feng, J. Wu, Y. Li, R. Gao, Z. Li, Q. Wang, Y. Cheng, B. He, Bioinspired soft microrobots with precise magneto-collective control for microvascular thrombolysis. Adv. Mater. 32, e2000366 (2020).
16. J. Shao, M. Xuan, H. Zhang, X. Lin, Z. Wu, Q. He, Chemotaxis-guided hybrid neutrophil micromotors for targeted drug transport. Angew. Chem. Int. Ed. Engl. 56, 12935-12939 (2017).
17. M. Wan, Q. Wang, R. Wang, R. Wu, T. Li, D. Fang, Y. Huang, Y. Yu, L. Fang, X. Wang, Y. Zhang, Z. Miao, B. Zhao, F. Wang, C. Mao, Q. Jiang, X. Xu, D. Shi, Platelet-derived porous nanomotor for thrombus therapy. Sci. Adv. 6, eaaz9014 (2020).
18. L. Wang, J. Wang, J. Hao, Z. Dong, J. Wu, G. Shen, T. Ying, L. Feng, X. Cai, Z. Liu, Y. Zheng, Guiding drug through interrupted bloodstream for potentiated thrombolysis by C-shaped magnetic actuation system in vivo. Adv. Mater. 33, e2105351 (2021).
19. B. Wang, K. Chan, J. Yu, Q. Wang, L. Yang, P. Chiu et al., Reconfigurable swarms of ferromagnetic colloids for enhanced local hyperthermia. Adv. Funct. Mater. 28, 1705701 (2018).
20. V. Iacovacci, L. Ricotti, E. Sinibaldi, G. Signore, F. Vistoli, A. Menciassi, An intravascular magnetic catheter enables the retrieval of nanoagents from the bloodstream. Adv. Sci. 5, 1800807 (2018).
21. Z. Wu, Y. Chen, D. Mukasa, O. S. Pak, W. Gao, Medical micro/nanorobots in complex media. Chem. Soc. Rev. 49, 8088-8112 (2020).
22. Z. Wu, L. Li, Y. Yang, P. Hu, Y. Li, S. Yang, L. V. Wang, W. Gao, A microrobotic system guided by photoacoustic computed tomography for targeted navigation in intestines in vivo. Sci. Robot. 4, aax0613 (2019).
23. J. F. Yu, B. Wang, X. Z. Du, Q. Q. Wang, L. Zhang, Ultra-extensible ribbon-like magnetic microswarm. Nat. Commun. 9, 3260 (2018).
24. D. Jin, K. Yuan, X. Du, Q. Wang, S. Wang, L. Zhang, Domino reaction encoded heterogeneous colloidal microswarm with on-demand morphological adaptability. Adv. Mater. 33, e2100070 (2021).
25. Y. Alapan, U. Bozuyuk, P. Erkoc, A. C. Karacakol, M. Sitti, Multifunctional surface microrollers for targeted cargo delivery in physiological blood flow. Sci. Robot. 5, aba5726 (2020).
26. I. C. Yasa, H. Ceylan, U. Bozuyuk, A. M. Wild, M. Sitti, Elucidating the interaction dynamics between microswimmer body and immune system for medical microrobots.

Sci. Robot. 5, eaaz3867 (2020).
27. J. Zhang, Z. Ren, W. Hu, R. H. Soon, I. C. Yasa, Z. Liu, M. Sitti, Voxelated three-dimensional miniature magnetic soft machines via multimaterial heterogeneous assembly. Sci. Robot. 6, abf0112 (2021).
28. H. Ceylan, N. O. Dogan, I. C. Yasa, M. N. Musaoglu, Z. U. Kulali, M. Sitti, 3D printed personalized magnetic micromachines from patient blood-derived biomaterials. Sci. Adv. 7, eabh0273 (2021).
29. S. Wang, X. Guo, W. Xiu, Y. Liu, L. Ren, H. Xiao, F. Yang, Y. Gao, C. Xu, L. Wang, Accelerating thrombolysis using a precision and clot-penetrating drug delivery strategy by nanoparticle-shelled microbubbles. Sci. Adv. 6, aaz8204 (2020).
30. Q. Wang, K. F. Chan, K. Schweizer, X. Du, D. Jin, S. C. H. Yu, B. J. Nelson, L. Zhang, Ultrasound Doppler-guided real-time navigation of a magnetic microswarm for active endovascular delivery. Sci. Adv. 7, abe5914 (2021).
31. Q. Wang, X. Du, D. Jin, L. Zhang, Real-time ultrasound Doppler tracking and autonomous navigation of a miniature helical robot for accelerating thrombolysis in dynamic blood flow. ACS Nano 16, 604-616 (2022).
32. W. Cao, Y. Liu, P. Ran, J. He, S. Xie, J. Weng, X. Li, Ultrasound-propelled janus rod-shaped micromotors for site-specific sonodynamic thrombolysis. ACS Appl. Mater. Interfaces 13, 58411-58421 (2021).
33. H. Engelhard, S. Pernal, Z. Gaertner, A. Levin, Y. Pan, S. Morris, M. E. Sabo, F. M. Creighton, A novel tissue culture tray for the study of magnetically induced rotation and translation of iron oxide nanoparticles. IEEE Magn. Lett. 8, 1-5 (2017).
34. T. O. Tasci, D. Disharoon, R. M. Schoeman, K. Rana, P. S. Herson, D. W. M. Marr, K. B. Neeves, Enhanced fibrinolysis with magnetically powered colloidal microwheels. Small 13, 1700954 (2017).
35. Y. Tao, X. Li, Z. Wu, C. Chen, K. Tan, M. Wan, M. Zhou, C. Mao, Nitric oxide-driven nanomotors with bowl-shaped mesoporous silica for targeted thrombolysis. J. Colloid Interface Sci. 611, 61-70 (2022).
36. J. Zheng, R. Qi, C. Dai, G. Li, M. Sang, Enzyme catalysis biomotor engineering of neutrophils for nanodrug delivery and cell-based thrombolytic therapy. ACS Nano 16, 2330-2344 (2022).
37. D. Fang, T. Li, Z. Wu, Q. Wang, M. Wan, M. Zhou, C. Mao, Dual drive mode polydopamine nanomotors for continuous treatment of an inferior vena cava thrombus. J. Mater. Chem. B 9, 8659-8666 (2021).
38. Q. Wang, B. Wang, J. Yu, K. Schweizer, B. J. Nelson, L. Zhang, “Reconfigurable magnetic microswarm for thrombolysis under ultrasound imaging” in 2020 IEEE International Conference on Robotics and Automation (ICRA) (IEEE, 2020), pp. 10285-10291.
39. L. Yang, J. Yu, S. Yang, B. Wang, B. J. Nelson, L. Zhang, A survey on swarm microrobotics. IEEE Trans. Robot. 38, 1531-1551 (2022).
40. J. Yu, T. Xu, Z. Lu, C. I. Vong, L. Zhang, On-demand disassembly of paramagnetic nanoparticle chains for microrobotic cargo delivery. IEEE Trans. Robot. 33, 1213-1225 (2017).
41. C. Wilhelm, J. Browacys, A. Ponton, J.-C. Bacri, Rotational magnetic particles microrheology: The maxwellian case. Phys. Rev. E Stat. Nonlin. Soft Matter Phys. 67, 011504 (2003).
42. S. Melle, O. G. Calderón, M. A. Rubio, G. G. Fuller, Microstructure evolution in magnetorheological suspensions governed by mason number. Phys. Rev. E Stat. Nonlin. Soft Matter Phys. 68, 041503 (2003).
43. A. J. Goldman, R. G. Cox, H. Brenner, Slow viscous motion of a sphere parallel to a plane wall-I Motion through a quiescent fluid. Chem. Eng. Sci. 22, 637-651 (1967).
44. G. Wang, X. Li, X. Tan, S. Ji, J. Huang, H. Duan, P. Lv, Motion enhancement of spherical surface walkers with microstructures. Adv. Intell. Syst. 3, 2000226 (2021).
45. J. C. K. Kwok, W. Huang, W. Leung, S. Chan, K. Chan, K. Leung, A. C. H. Chu, A. K. N. Lam, Human placenta as an ex vivo vascular model for neurointerventional research. J. NeuroInterv. Surg. 6, 394-399 (2021).
46. H. Deng, X. L. Li, Q. Peng, X. Wang, J. P. Chen, Y. D. Li, Monodisperse magnetic single-crystal ferrite microspheres. Angew. Chem. Int. Ed. Engl. 44, 2782-2785 (2005).

What is claimed is:

1. A nanobot for recanalization of a bodily tube, comprising:
a. A magnetic core;
b. A mesoporous shell coated on said magnetic core; and
c. An outer layer of a chemical for recanalization bound to said mesoporous shell;
wherein said nanobot is adapted to respond to an external magnetic field to carry out motion for mechanical and chemical recanalization.

2. The nanobot of claim 1, wherein said chemical for recanalization comprises thrombolytic agents.

3. The nanobot of claim 1, wherein said chemical for recanalization comprises tissue plasminogen activator.

4. The nanobot of claim 1, wherein said magnetic core comprises one or more of Fe, Co, Ni, Mn, and their oxides or alloys.

5. The nanobot of claim 1, wherein said magnetic core comprises Fe₃O₄.

6. The nanobot of claim 1, wherein said mesoporous shell is made of a material comprising metal-organic framework (MOF) or cell membrane.

7. The nanobot of claim 1, wherein said mesoporous shell comprises silica, Zeolitic Imidazolate Framework-8, Red Blood Cell Membrane or polydopamine.

8. The nanobot of claim 1, wherein said external magnetic field comprises one or more selected from the group consisting of static magnetic field, tumbling magnetic field, rotating magnetic field, oscillating magnetic field, conical magnetic field, and gradient magnetic field.

9. The nanobot of claim 1, wherein said motion comprises staying stationary, rotating, or moving along a desired trajectory.

10. The nanobot of claim 1, wherein said nanobot has a size range of 20-5000 nm.

11. The nanobot of claim 1, wherein said nanobot has a size of about 300 nm.

12. A system for recanalization of a bodily tube, comprising:
a. A plurality of the nanobots of claim 1;
b. A delivery mechanism for delivering said plurality of nanobots to said bodily tube; and
c. A magnetic source adapted to provide an external magnetic field for controlling motion of said plurality of nanobots.

13. The system of claim 12, said delivery mechanism is a catheter comprising a tip adapted to store and release said plurality of nanobots.

14. The system of claim 12, said external magnetic field comprises one or more selected from the group consisting of static magnetic field, tumbling magnetic field, rotating magnetic field, oscillating magnetic field, conical magnetic field, and gradient magnetic field.

15. The system of claim 12, further comprising a flow-reducing mechanism for blocking or reducing the flow rate to the said bodily tube.

16. A method for recanalization of a bodily tube using the system of claim 12, comprising the steps of:
a. Introducing said plurality of nanobots to vicinity of a recanalization site within said bodily tube using said delivery mechanism;
b. Directing said plurality of nanobots to contact said recanalization site using from said magnetic source; and
c. Allowing said plurality of nanobots to mechanically interact with said recanalization site in a rotating magnetic field from said magnetic source.

17. The method of claim 16, further comprising the step of retrieving said plurality of nanobots after step (c).

18. The method of claim 16, further comprising the step of reducing flow or blocking said bodily tube prior to introducing said plurality of nanobots in step (a).

19. The method of claim 16, wherein said bodily tube comprises one or more selected from the group consisting of blood vessels, vas deferens, and gland ducts.
